(19) United States
(12) Patent Application Publication
(10) Pub. No.: US 2010/0172841 A1
(43) Pub. Date: Jul. 8, 2010

(54) VIABLE NEAR-INFRARED FLUOROCHROME LABELED CELLS AND METHODS OF MAKING AND USING THE SAME

(76) Inventors: Jeffrey D. Peterson, Shrewsbury, MA (US); Milind Rajopadhye, Westford, MA (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/555,754

(22) Filed: Sep. 8, 2009

Related U.S. Application Data
(63) Continuation-in-part of application No. PCT/US2008/056235, filed on Mar. 7, 2008.
(60) Provisional application No. 60/905,673, filed on Mar. 8, 2007.

(51) Int. Cl. A61K 49/00; C07H 15/00; C12N 5/07

(52) U.S. Cl. 424/9.2; 424/9.6; 435/325; 435/372.2; 435/372.3; 435/366

(57) ABSTRACT

The invention provides viable near-infrared fluorochrome labeled cells and in vivo imaging methods for tracking, locating or determining the quantity of the viable cells once they have been administered to a subject.

### RELATED APPLICATIONS

This application is a continuation-in-part of International Patent Application No. PCT/US2008/056235, filed Mar. 7, 2008, which claims the benefit of and priority to U.S. Patent Application Ser. No. 60/905,673, filed Mar. 8, 2007, the entire disclosures of each of which are incorporated by reference herein for all purposes.

### BACKGROUND

Optical imaging methods offer a number of advantages over other imaging methods. Such imaging typically uses light in the red and near-infrared (NIR) range (600-1200 nm) to maximize tissue penetration and minimize absorption from natural biological absorbers such as hemoglobin and water. Optical imaging may provide high sensitivity, does not require exposure of test subjects or laboratory personnel to ionizing radiation, can allow for simultaneous use of multiple, distinguishable probes (which may be important in molecular imaging), and offers high temporal and spatial resolution, which is important in functional imaging and in vivo microscopy, respectively.

In fluorescence imaging, filtered light or a laser with a defined bandwidth is used as a source of excitation light. The excitation light travels through body tissue, and when the excitation light encounters a reporter molecule (for example, a contrast agent or imaging probe), the light is absorbed. The reporter molecule then emits light that has detectably different properties from the excitation light. The resulting emitted fluorescent light then can be used to construct an image.

The tracking of cells in intact micro- and macroenvironments over time in vivo has been a long cherished goal in understanding mechanism and function of different cell types, including the role of different cell types in disease development. In vivo fluorescent imaging techniques currently include imaging cells that express a recombinant light generating molecule, for example, a fluorescent protein or luciferase. In these techniques, cells express a bioluminescent reporter gene encoding the light generating moiety under a specific promoter. These types of techniques permit in vivo optical imaging; however, since it requires genetic manipulation of the cells, this approach is not suitable for labeling primary cells, cells in situ, or for human clinical applications.

Fluorescent dyes are generally known and have been used for fluorescence labeling and detection of cells in vitro in applications such as microscopy and flow cytometry. However, fluorescent dyes and associated in vivo imaging methods for cell localization and tracking have not been well established.

Thus, there is an ongoing need for new fluorescent dyes and associated in vivo imaging methods for cell tracking and localization that can be used in various medical, diagnostic and biological applications.

### SUMMARY OF THE INVENTION

The invention is based, in part, upon the discovery, that it is possible to label, for example, covalently label, viable cells, for example, mammalian cells, with a near-infrared fluorochrome such that the cells remain viable after labeling. The resulting labeled cells can then be used in a variety of imaging methods, and are a particularly useful for in vivo imaging.

In one aspect, the invention provides an in vivo imaging method for tracking and/or locating and/or determining a quantity of viable cells in a subject, for example, a mammal, for example, a human. The method comprises the steps of: (a) administering, for example, systemically or locally, to the subject a plurality of viable cells covalently labeled with at least one near-infrared fluorochrome, wherein at least 50% of the cells remain viable after labeling; (b) directing near-infrared excitation light into the subject; and (c) detecting fluorescent light emitted from the cells thereby to track and/or locate and/or determine a quantity of the cells in the subject. It is contemplated, however, that steps (b) and (c) can be repeated at discrete or continuous points in time.

The method optionally further comprises processing the detected fluorescent light emitted from the cells to create an image representation, for example, a tomographic image, of a region within the subject. The representation can be co-registered with an image of the subject or a region within the subject obtained by X-ray, magnetic resonance, computed tomography, ultrasound, single photon emission tomography, or positron emission tomography.

The near-infrared fluorochrome can be a carbocyanine dye (for example, an indocyanine dye), that optionally comprises a functional group, for example, a succinimidyl ester, that facilitates covalent linkage to a cellular component. Exemplary dyes include, for example, Cy5, Cy5.5, and Cy7, each of which are available from GE Healthcare; VivoTag-680, VivoTag-S680, VivoTag-S750, each of which are available from VisEn Medical; AlexaFluor660, AlexaFluor680, AlexaFluor700, AlexaFluor750, and Alexa Fluor790, each of which are available from Invitrogen; Dy677, Dy676, Dy682, Dy752, Dy780, each of which are available from Dyonics; DyLight547 and DyLight647, each of which are available from Pierce; HiLyte Fluor 647, HiLyte Fluor 680, and HiLyte Fluor 750, each of which are available from AnaSpec; IRDye800CW, IRDye 800RS, and IRDye 700DX, each of which are available from Li-Cor; and ADS780WS, ADS830WS, and ADS832WS, each of which are available from American Dye Source.

In certain embodiments, the near-infrared fluorochrome used to label the cells is selected from the group consisting of:

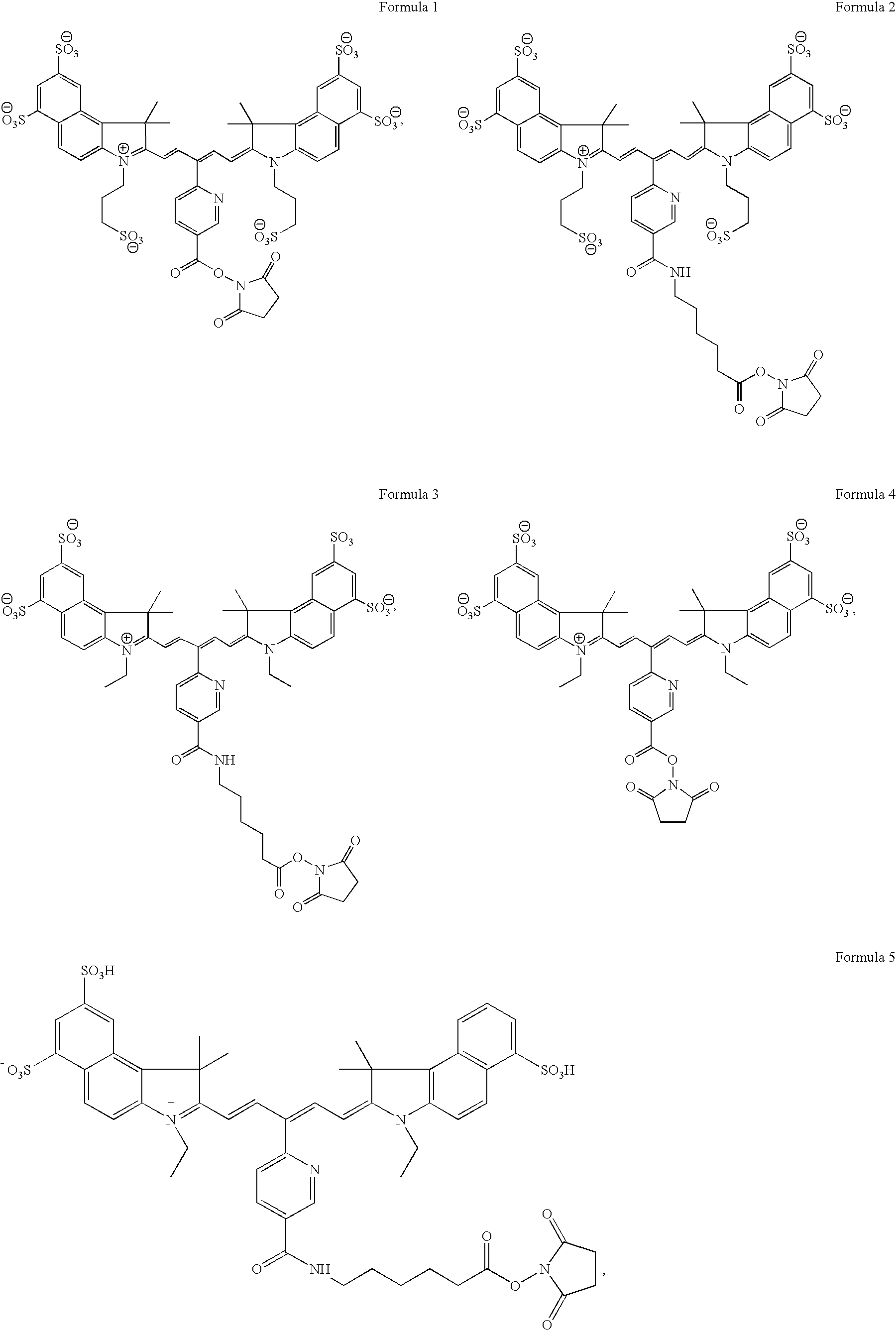

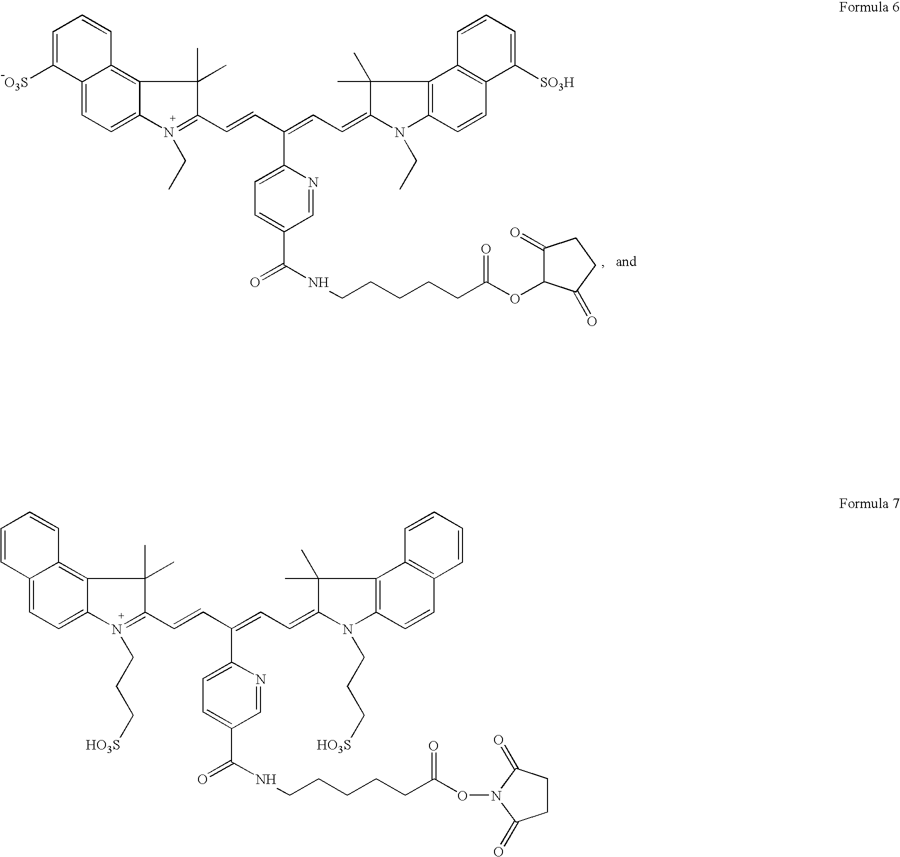

In certain embodiments, the near infrared fluorochrome used to label the cells is:

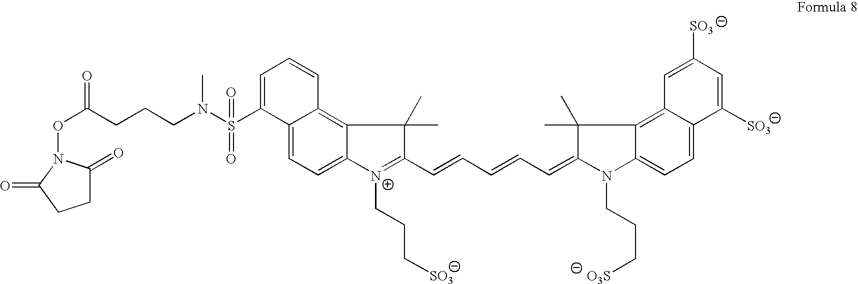

In certain embodiments, the near infrared fluorochrome used to label the cells is selected from the group consisting of:

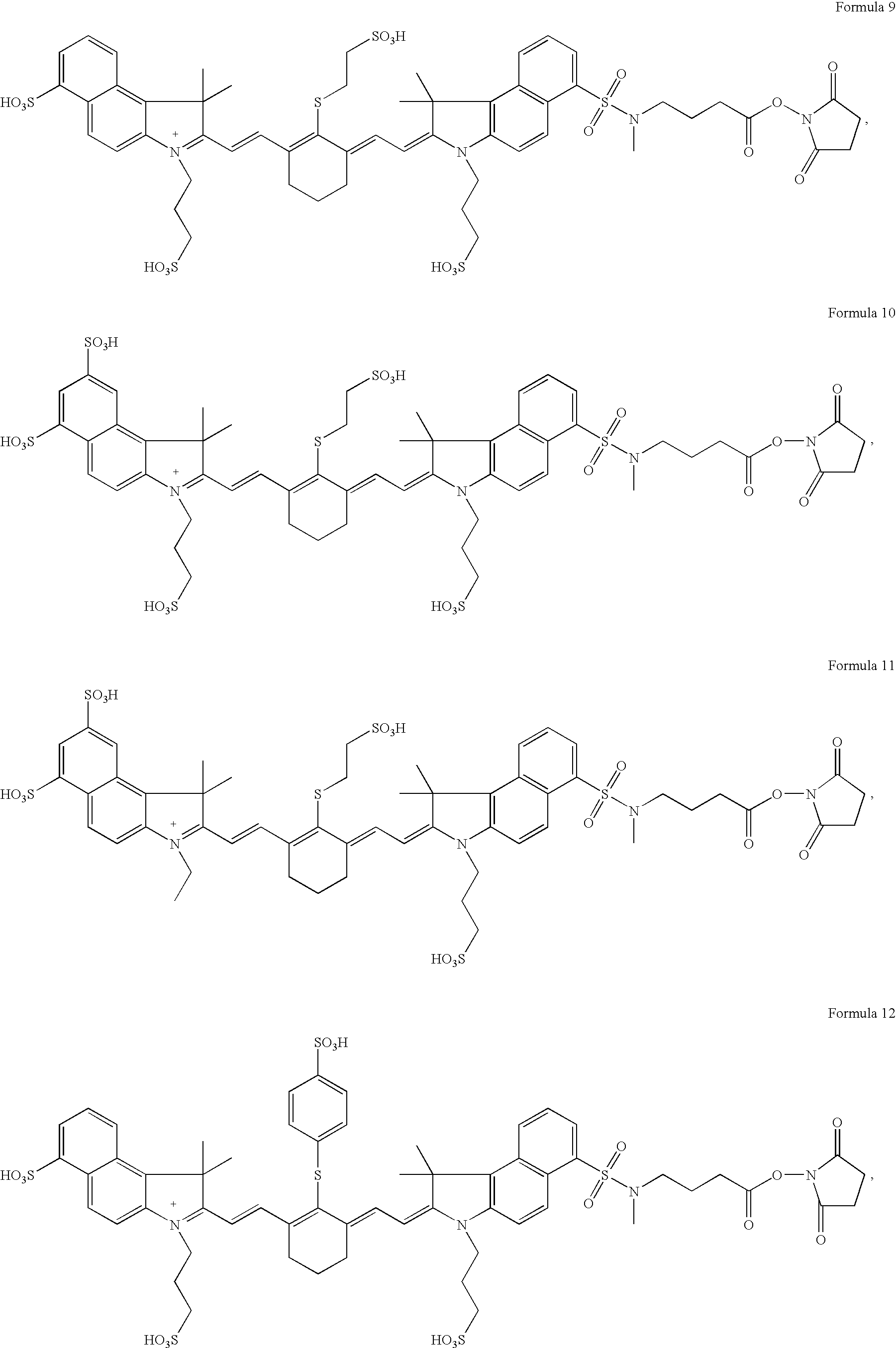

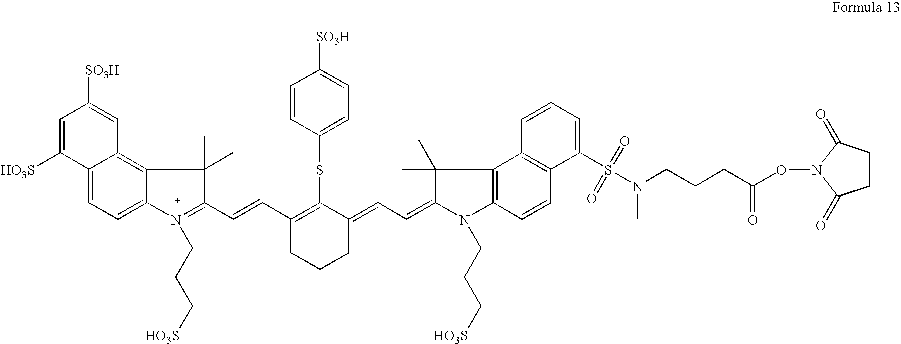

In certain embodiments, the near infrared fluorochrome used to label the cells is selected from the group consisting of:

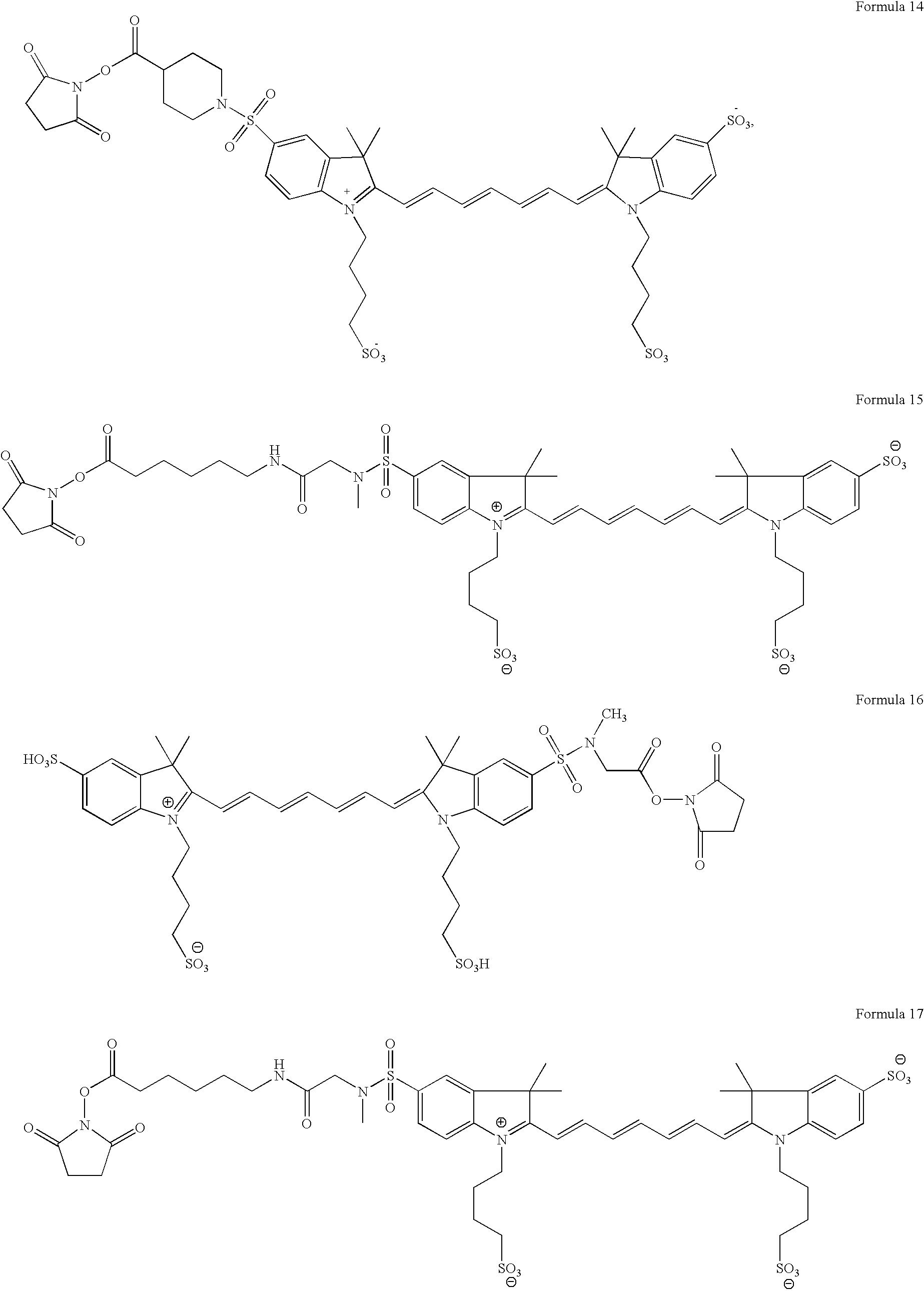

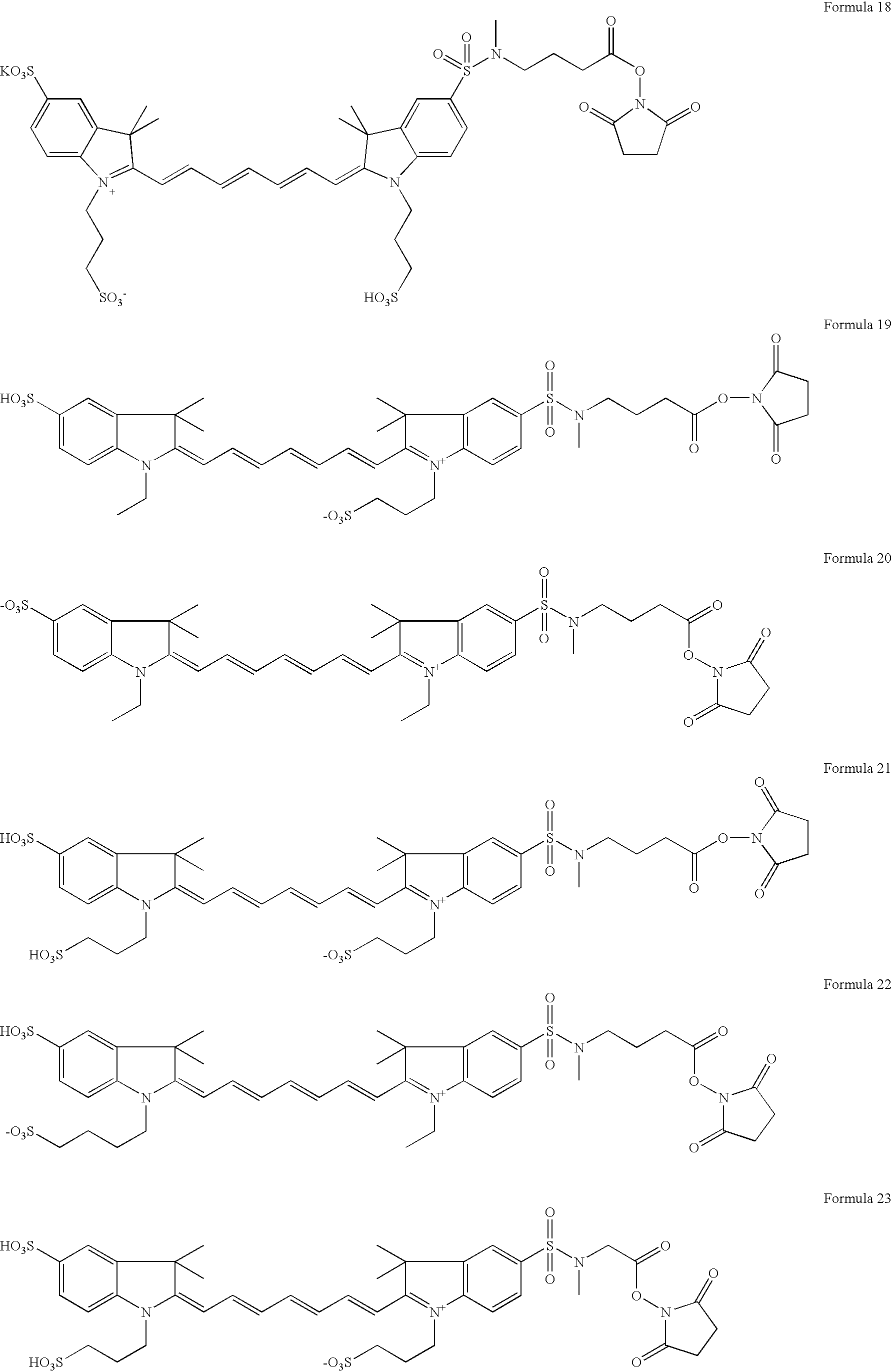

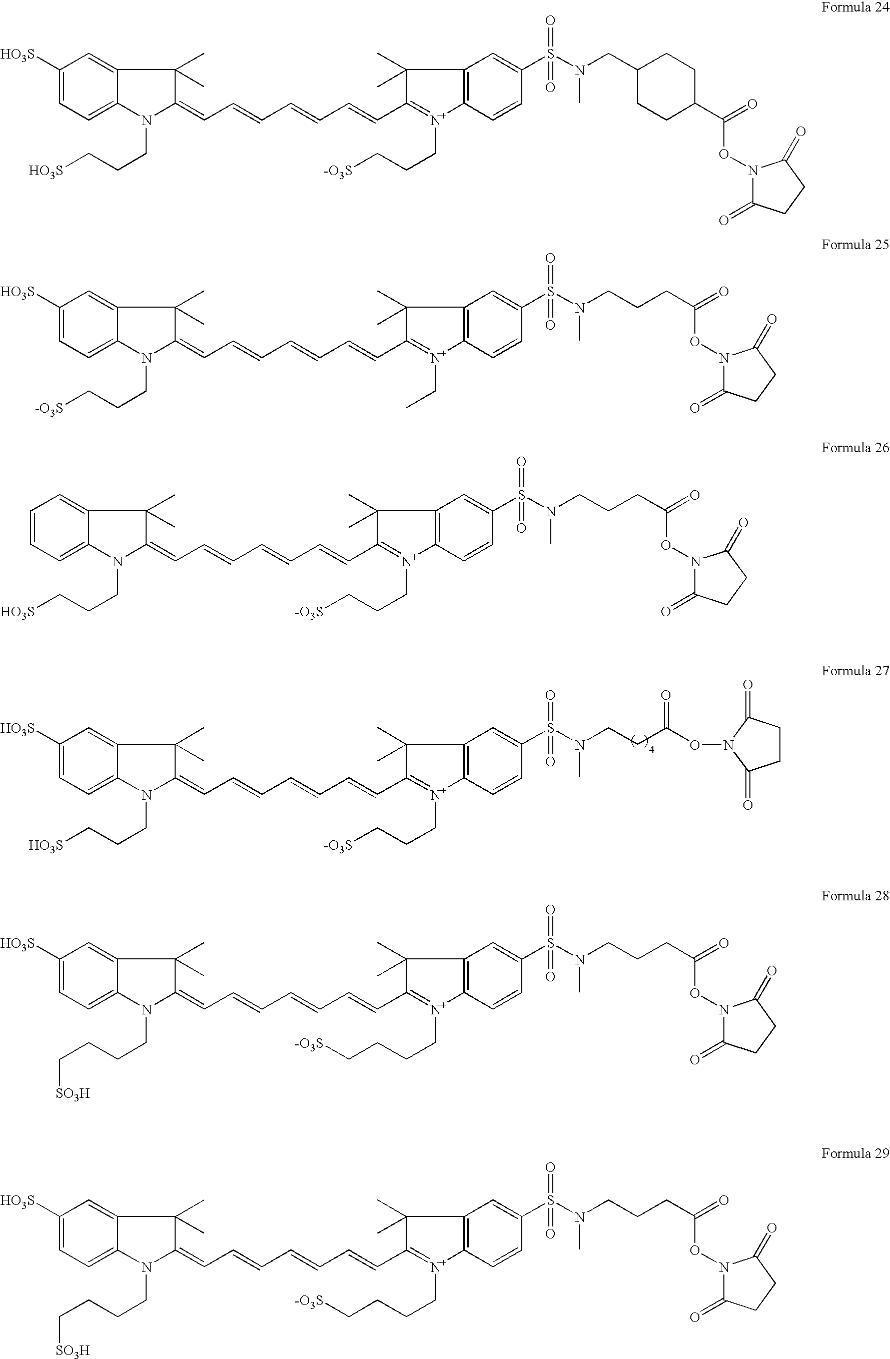

In certain embodiments, the near infrared fluorochrome used to label the cells is selected from the group consisting of:

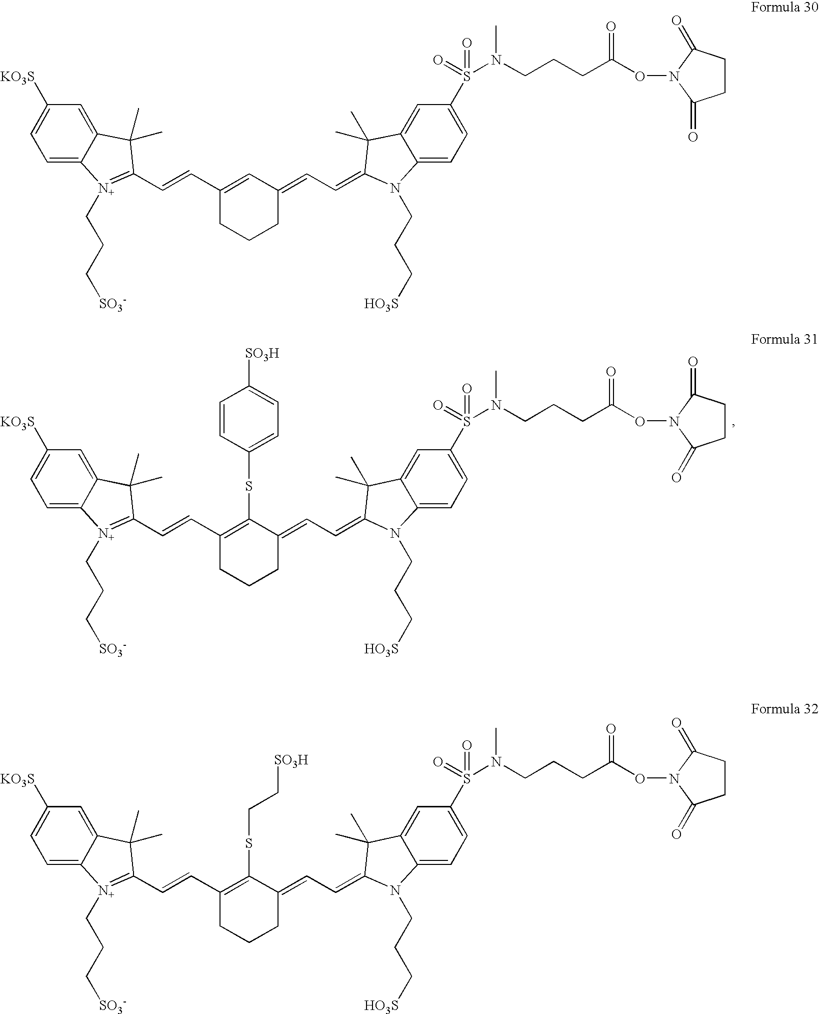

It is understood that the viable cells can be primary cells. The viable cells can be selected from the group consisting of T-cells, B-cells, tumor cells, stem cells, bacterial cells, macrophages, lymphocytes, monocytes and other immune cells. The near-infrared fluorochrome can be covalently linked to a component of the cell, for example, a reactive amine in an amino acid residue, via a chemical reactive functional group on the fluorochrome. Exemplary chemically reactive functional groups include, for example, a succinimidyl ester moiety (for example, an amine reactive N-hydroxysuccinimide (NHS) ester), tetrafluorophenyl ester, pentafluorophenyl ester, para-nitrophenyl ester, benzotriazolyl ester, aldehyde, and an iodoacetyl group.

In the method, steps (b) and/or (c) can be performed using at least one of: an endoscope, catheter, planar system, reflectance system, tomographic system, optical imaging system and/or an intraoperative microscope. Furthermore, the resulting representations can be co-registered with an image of the subject or a region within the subject obtained by X-ray, magnetic resonance, computed tomography, ultrasound, single photon emission tomography, or positron emission tomography

The method can be used to detect and/or monitor the development or regression of a disease. Exemplary diseases include bone disease, cancer, cardiovascular disease, environmental disease, dermatological disease, immunologic disease, inherited disease, infectious disease, inflammatory disease, metabolic disease, neurodegenerative disease, ophthalmic disease, and respiratory disease. Furthermore, the method can be used to detect and/or monitor cell-based therapies.

In another aspect, the invention provides a method of making a plurality of viable near-infrared fluorochrome labeled cells for use in in vivo imaging. The method comprises: (a) contacting a plurality of viable cells with near-infrared fluorochrome molecules under conditions (i) to covalently link the cells with at least one near-infrared fluorochrome, and (ii) to maintain the viability of the cells, wherein the cells have substantially the same function and/or viability as the cells prior to labeling; and (b) removing unbound near-infrared fluorochrome molecules, thereby to produce a plurality of viable near-infrared fluorochrome labeled cells. Step (a) can be performed such that the reaction occurs in a solution substantially free (for example, less than 2% (v/v), 1% (v/v), 0.5% (v/v), 0.1% (v/v), 0.05% (v/v)) of organic solvent, for example, DMSO.

In another aspect, the invention provides compositions for use in in-vivo imaging comprising a plurality of viable cells, for example, primary cells, covalently linked to at least one near-infrared fluorochrome molecule, wherein the cells have substantially the same function and/or viability as the cells prior to labeling. The cells can be selected from the group consisting of B-cells, T-cells, tumor cells, stem cells, bacterial cells, macrophages, lymphocytes, monocytes and other immune cells.

In one embodiment, the near-infrared fluorochrome molecule is a carbocyanine dye, for example, an indocarbocyanine cell, optionally comprising a succinimidyl ester moiety. In certain embodiments, the near-infrared fluorochrome molecule is selected from the group consisting of Cy5, Cy5.5, Cy7, VivoTag-680, VivoTag-5680, VivoTag-5750, AlexaFluor660, AlexaFluor680, AlexaFluor700, AlexaFluor750, Dy677, Dy676, Dy682, Dy752, Dy780, DyLight547, and DyLight647.

In one embodiment, the near-infrared fluorochrome molecule used to label the cells is a compound selected from the group consisting of:

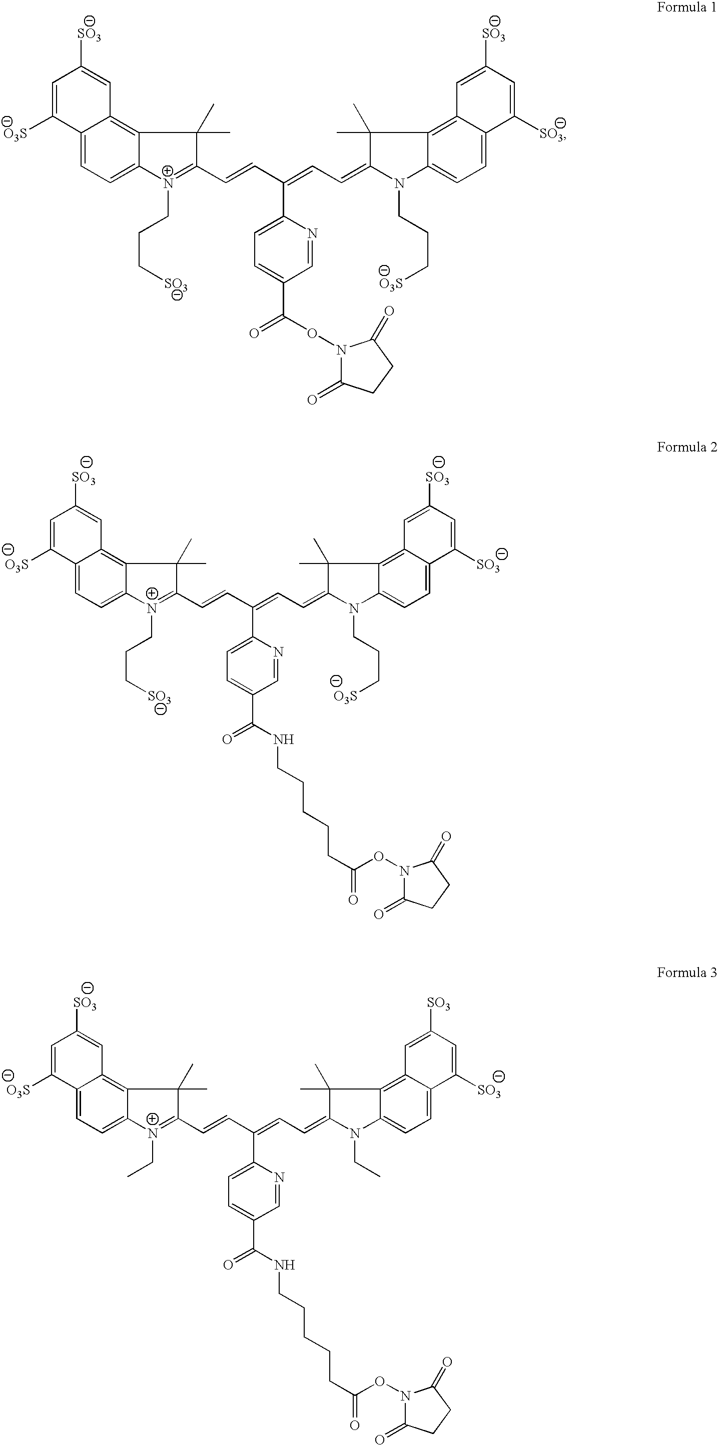

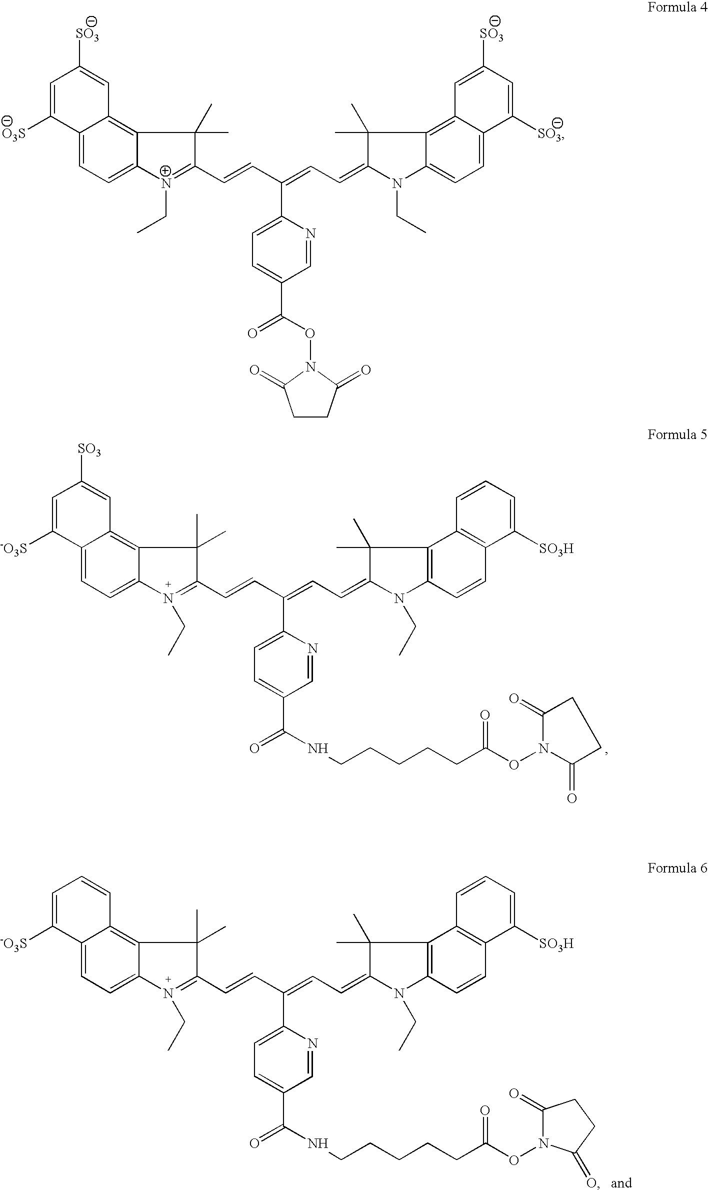

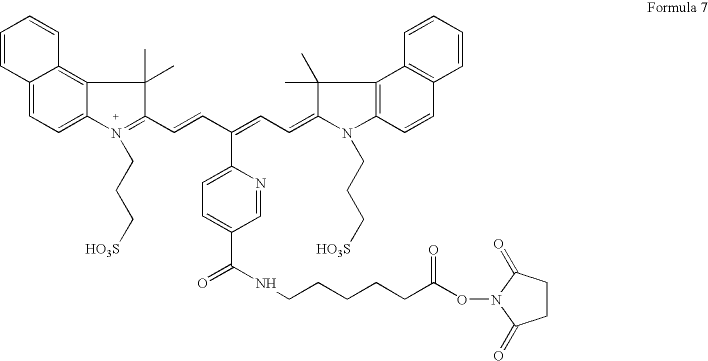

In one embodiment, the near infrared fluorochrome molecule used to label the cells is:

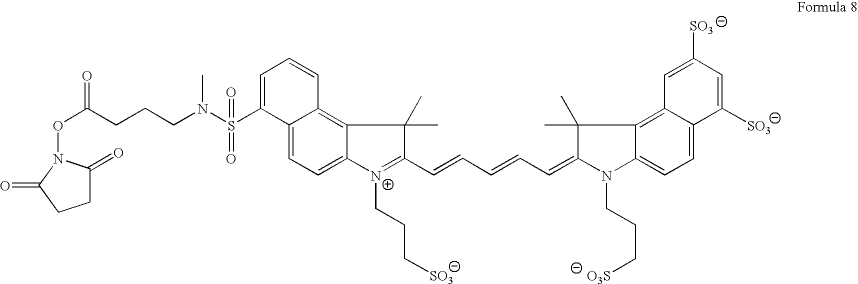

In one embodiment, the near infrared fluorochrome molecule used to label the cells is a compound selected from the group consisting of:

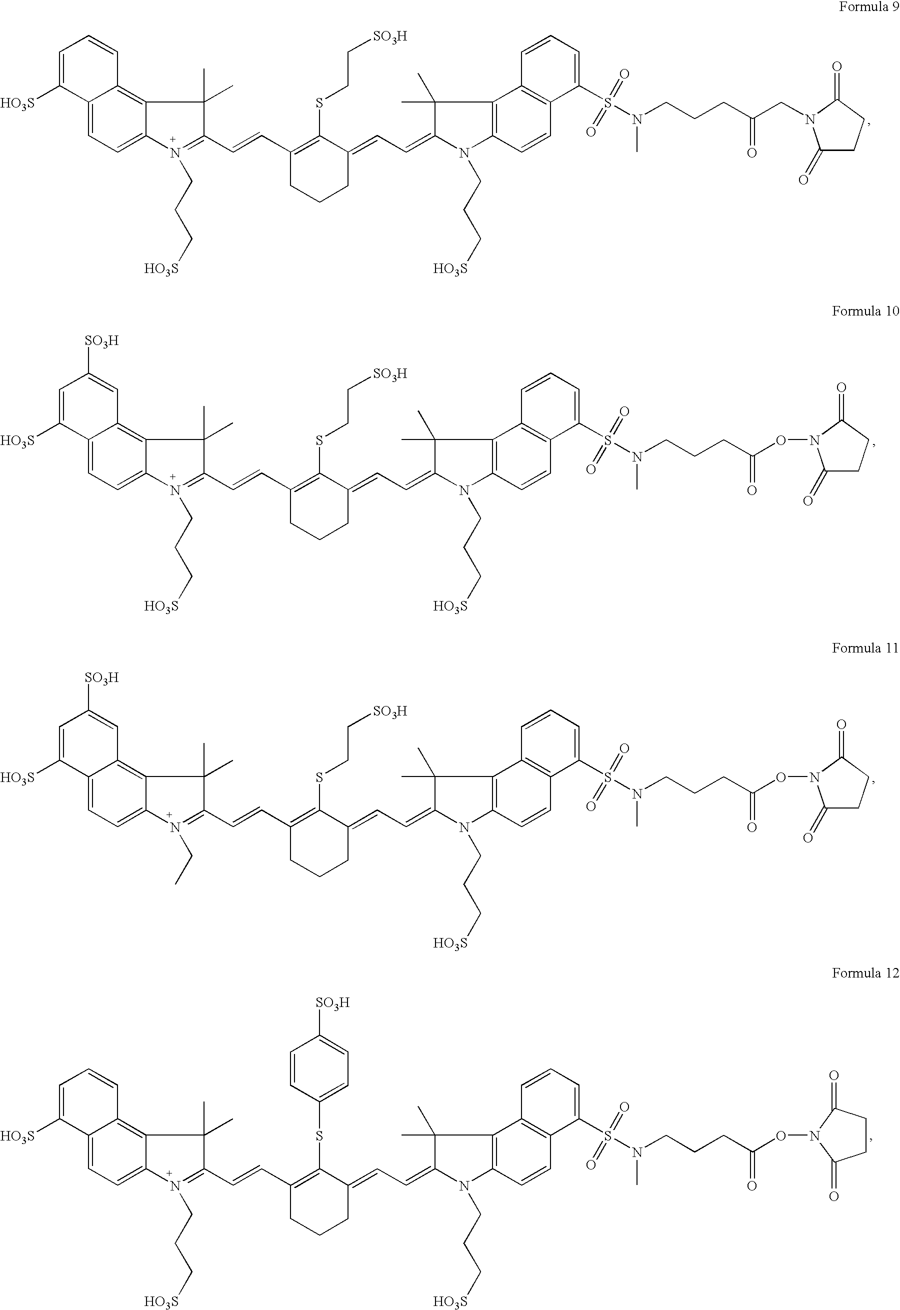

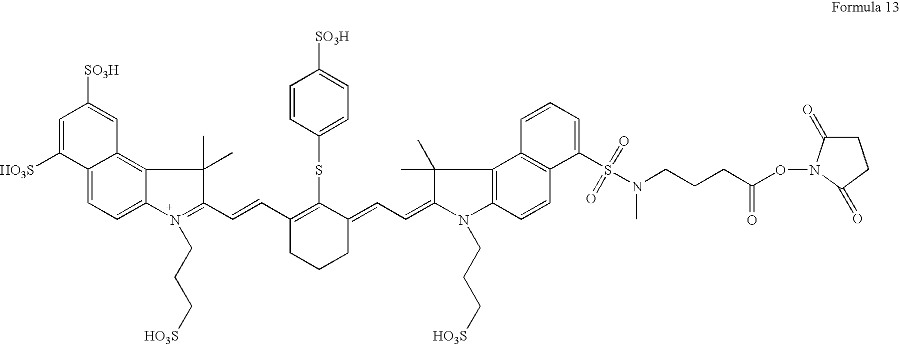

In one embodiment, the near infrared fluorochrome molecule used to label the cells is a compound selected from the group consisting of:

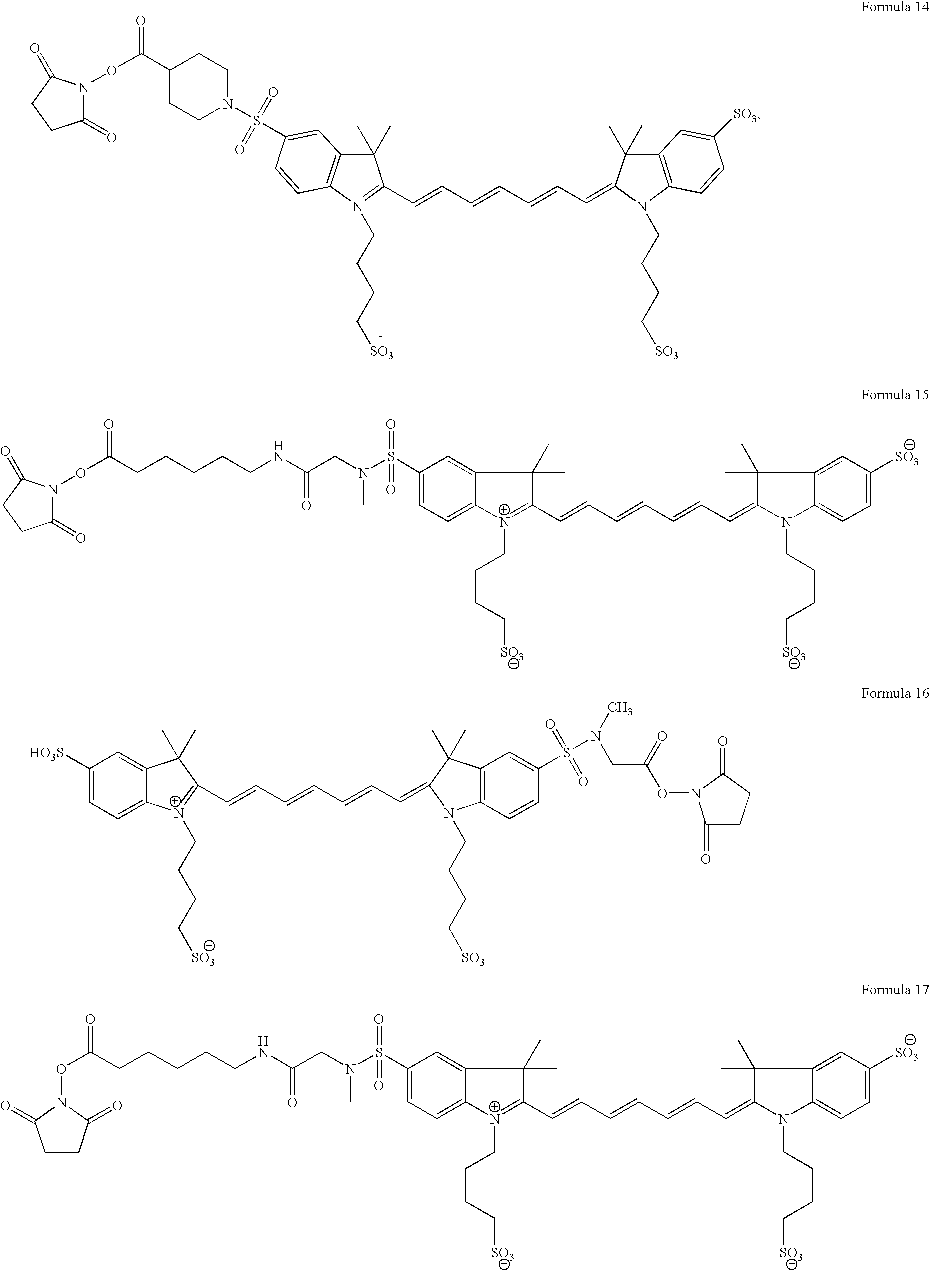

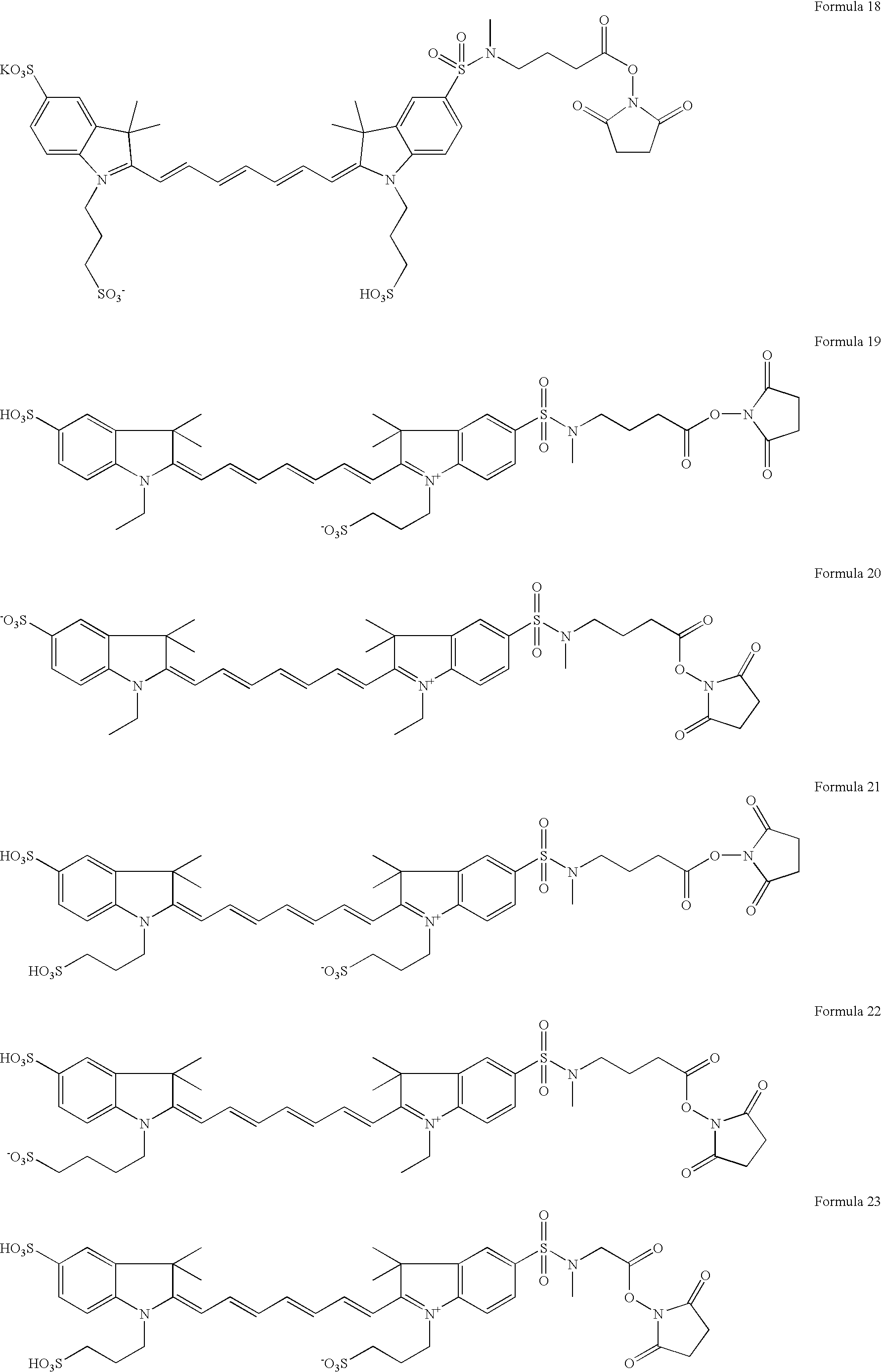

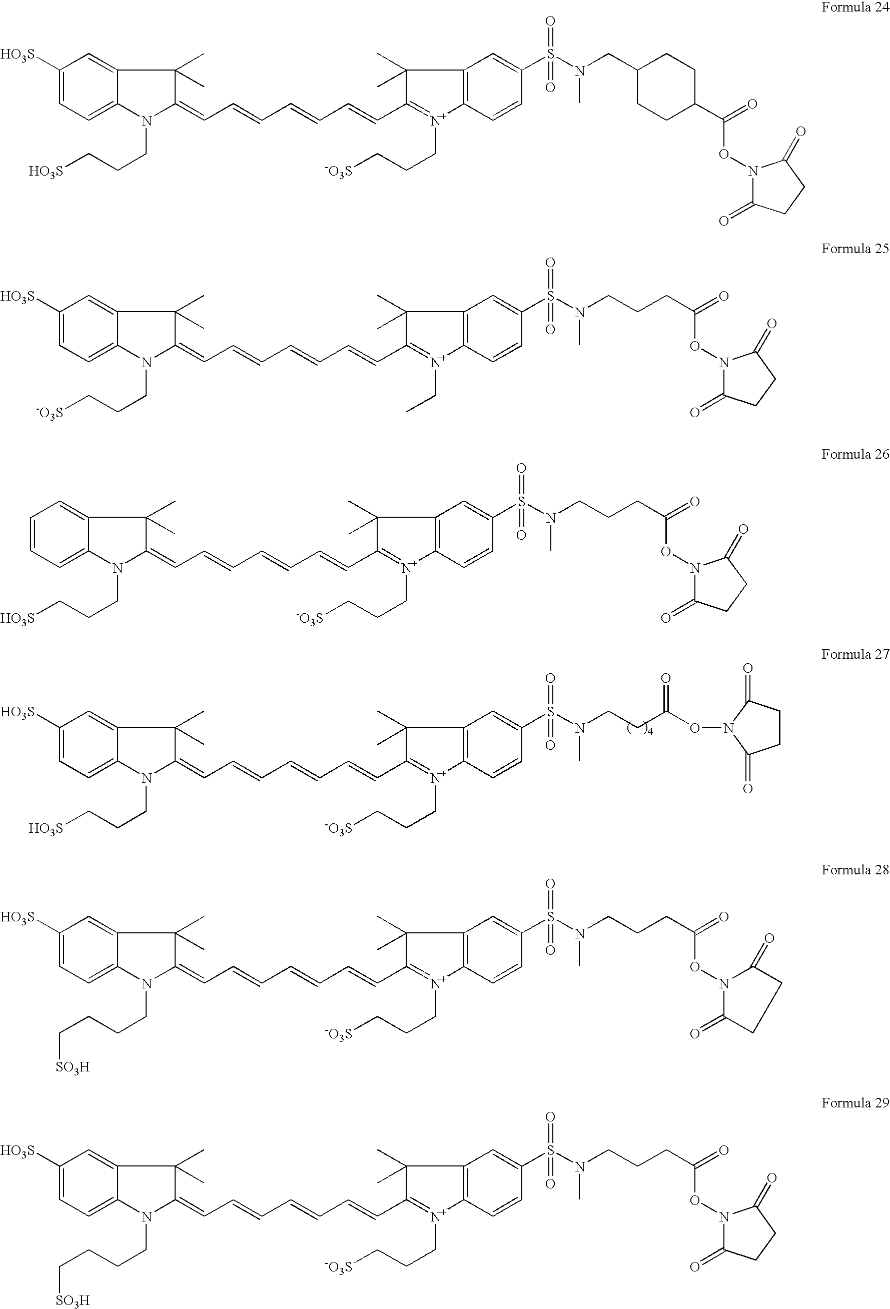

In one embodiment, the near infrared fluorochrome molecule used to label the cells is a compound selected from the group consisting of:

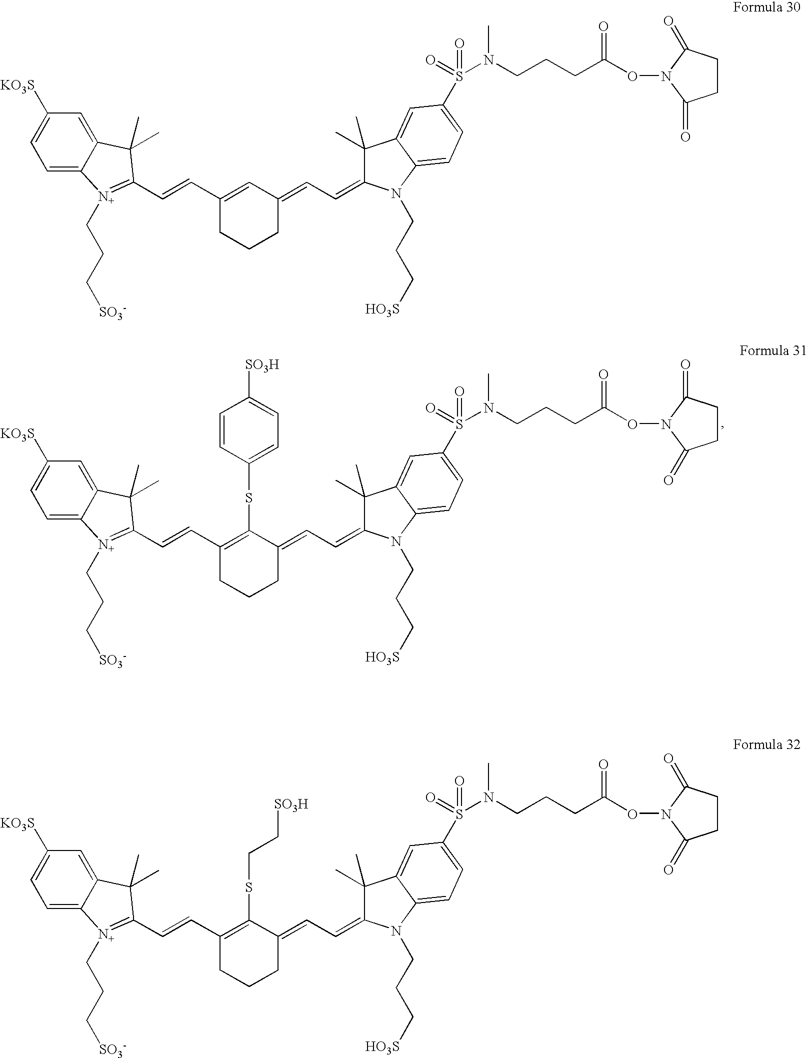

The foregoing compositions (Formulas 1-32) optionally are substantially free (for example, less than 2% (v/v), 1% (v/v), 0.5% (v/v), 0.1% (v/v), 0.05% (v/v)) of an organic solvent, for example, DMSO. Under certain circumstances, for example, when the labeling occurs under conditions substantially free of an organic solvent, for example, DMSO, the resulting labeled cells have substantially the same function and/or viability as the cells prior to labeling.

In another aspect, the invention relates to the use of a plurality of viable cells, for example, mammalian cells, each associated, for example, covalently associated, with at least one near-infrared fluorochrome molecule selected from the group consisting of: Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, and DyLight647, HiLyte Fluor 680, HiLyte Fluor 750, IRDye800CW, IRDye 800RS, IRDDye 700DX, ADS780WS, ADS832WS, and at least one of Formula 1-Formula 32, in the preparation of an agent for use in in vivo near-infrared imaging. The viable cells can be primary cells. The cells can be selected from a group consisting of B-cells, T-cells, immune cells, tumor cells, stem cells, macrophages, lymphocytes, monocytes, and splenocytes.

### BRIEF DESCRIPTION OF THE DRAWINGS

The invention maybe more clearly understood by reference to the drawings in which,

FIG. 1 shows images of a mouse 30 minutes (FIG. 1A) and 6 days (FIG. 1B) after having received viable HT-29 cells labeled with the fluorochrome VivoTag680 (succinimidyl ester); and

FIG. 2 is a graph showing the change in fluorescence of the two tumors detected and shown in FIG. 1 as a function of time.

### DETAILED DESCRIPTION OF THE INVENTION

The invention relates to in vivo imaging compositions containing viable cells labeled with a near-infrared fluorochrome, and to methods for tracking and/or locating and/or determining a quantity of viable, labeled cells in a subject. In certain embodiments the near-infrared fluorochrome is covalently linked to a cellular component (for example, to a membrane, organelle, protein, peptide, sugar, saccharide, polysaccharide, lipid, glycolipid, glycoprotein, or nucleic acid).

The in vivo imaging methods comprise administering to the subject a plurality of viable near-infrared fluorochrome labeled cells; directing near-infrared excitation light into the subject; and detecting fluorescent light emitted from the cells thereby to track and/or locate and/or determine a quantity of the cells in the subject. The signal emitted by the labeled cells can be used to construct an image, for example, a tomographic image, of a region or structure to be imaged. Such steps can be repeated at, for example, predetermined time intervals thereby to permit evaluation of the emitted signals of the cells in the subject over time. The foregoing steps can be repeated at predetermined intervals thereby permitting the evaluation of the emitted signals of the cells in the subject over time. In certain embodiments, two or more near-infrared fluorochrome labeled cells whose signal properties are distinguishable can be administered to the subject and their emission properties can be used to image two or more cell types in the subject.

The in vivo imaging methods can be used to detect and/or monitor a disease, for example, bone disease, cancer, cardiovascular disease, dermatological disease, environmental disease, immunologic disease, infectious disease, inflammation, inherited disease, metabolic disease, neurodegenerative disease, ophthalmic disease, and respiratory disease. The signal emitted by cells can be used to monitor transport, trafficking, and localization of the cells or to evaluate the efficacy of a cell therapy.

The labeled cells can be derived directly from a subject (i.e., are autologous cells) or can be derived from another source (for example, from another subject, cell culture, etc.). The labeled cells preferably retain substantially all, or at least partial, viability and/or function as compared to an unlabeled cell.

The fluorescently labeled cells can be administered to the subject systemically, for example, by injection into the blood, or locally, for example, by locally injecting the cells into the subject.

The term, “cellular component,” as used herein refers to a component of a viable cell that becomes covalently linked to a fluorochrome. Exemplary cellular components include, for example, a protein, peptide, sugar, saccharide, polysaccharide, lipid, glycoprotein, glycolipid or a nucleic acid (for example, deoxyribonucleic acid or ribonucleic acid). Furthermore, the cellular component can be, for example, an organelle, or a membrane. Similarly, the cellular component can be, for example, an enzyme, receptor, ligand, hormone, etc.

The term, “fluorochrome,” as used herein refers to a fluorochrome, a fluorophore, a fluorescent organic or inorganic dye, a metal chelate that changes the fluorescence of any entity, or a fluorescent enzyme substrate (including protease activatable enzyme substrates).

The terms, “near-infrared fluorochrome or NIRF,” as used herein refer to fluorochromes with absorption and emission maximum between about 600 and about 1200 nm, more preferably between about 600 nm and about 900 nm. The NIRFs preferably have an extinction coefficient of at least 50,000 M⁻¹cm⁻¹ per fluorochrome molecule in aqueous medium. The NIRFs preferably also have (1) high quantum yield (i.e., quantum yield greater than 5% in aqueous medium), (2) narrow excitation/emission spectrum, spectrally separated absorption and excitation spectra (i.e., excitation and emission maxima separated by at least 15 nm), (3) high chemical and photostability, (4) nontoxicity, (5) good biocompatibility, biodegradability and excretability, and (6) commercial viability and scalable production for large quantities (i.e., gram and kilogram quantities) required for in vivo and human use.

In particular, certain carbocyanine, indocarbocyanine or polymethine fluorescent dyes can be used for labeling cells for use in the methods of the invention, and include those described, for example, in U.S. Pat. No. 6,747,159; U.S. Pat. No. 6,448,008; U.S. Pat. No. 6,136,612; U.S. Pat. Nos. 4,981,977; 5,268,486; U.S. Pat. No. 5,569,587; U.S. Pat. No. 5,569,766; U.S. Pat. No. 5,486,616; U.S. Pat. No. 5,627,027; U.S. Pat. No. 5,808,044; U.S. Pat. No. 5,877,310; U.S. Pat. No. 6,002,003; U.S. Pat. No. 6,004,536; U.S. Pat. No. 6,008,373; U.S. Pat. No. 6,043,025; U.S. Pat. No. 6,127,134; U.S. Pat. No. 6,130,094; U.S. Pat. No. 6,133,445; also WO 97/40104, WO 99/51702, WO 01/21624, and EP 1 065 250 A1; and Tetrahedron Letters 41, 9185-88 (2000).

In certain embodiments, the NIRF further comprises a functional group that reacts with a reactive group in a cellular component, for example, a primary amine, a sulfydryl group, to produce a covalent linkage between the NIRF and the cellular component. Exemplary functional groups include, for example, a succinimidyl ester moiety (for example, an amine reactive N-hydroxysuccinimide (NHS) ester), tetrafluorophenyl ester, pentafluorophenyl ester, para-nitrophenyl ester, benzotriazolyl ester, aldehyde, and an iodoacetyl group. Under certain circumstances, it has been found that when the functional group of the NIRF is cleaved or hydrolyzed (therefore, unavailable to form a covalent bond with a cellular component) under aqueous conditions the resulting cells are not as “bright” as when the NIRF contains the functional group.

It is believed that, under certain circumstances (for example, when particular NIRFs and cell types are chosen to produce labeled cells), a covalent linkage is necessary to produce a labeled cell that is both sufficiently viable and labeled to be useful in the in vivo imaging methods described herein. Under certain circumstances, the cells retain substantially the same function and/or viability as the cells prior to labeling. With regard to function, dependent upon the cell type of interest, it is understood that the labeled cells retain one or more of, for example, their ability to divide, proliferate, produce and/or secrete a molecule of interest, metabolize a molecule of interest, or bind to a solid support, tissue, cell or ligand of interest, each of which can be determined using techniques known in the art. With regard to viability, the viability of the labeled cells can be determined by techniques known in the art, for example, via a Trypan Blue exclusion assay (Cellgro Mediatech, Inc.). The labeled cells should not only be viable but also contain enough label to be visualized by an in vivo imaging protocol. Depending upon the labeling conditions, at least 50%, 60%, 70%, 80%, 90%, or 95% of the cells remain viable post labeling.

In certain embodiments, at least 50%, 60%, 70%, 80%, 90%, or 95% of the cells remain viable 1 hour after labeling. In certain embodiments, at least 50%, 60%, 70%, 80%, 90%, or 95% of the cells remain viable 3 hours after labeling. In certain embodiments, at least 50%, 60%, 70%, 80%, 90%, or 95% of the cells remain viable 6 hours after labeling. In certain embodiments, at least 50%, 60%, 70%, 80%, 90%, or 95% of the cells remain viable 9 hours after labeling. In certain embodiments, at least 50%, 60%, 70%, 80%, 90%, or 95% of the cells remain viable 12 hours after labeling. In certain embodiments, at least 50%, 60%, 70%, 80%, 90%, or 95% of the cells remain viable 18 hours after labeling. In certain embodiments, at least 50%, 60%, 70%, 80%, 90%, or 95% of the cells remain viable 24 hours after labeling. In certain embodiments, at least 50%, 60%, 70%, 80%, 90%, or 95% of the cells remain viable 36 hours after labeling. In certain embodiments, at least 50%, 60%, 70%, 80%, 90%, or 95% of the cells remain viable 48 hours after labeling. In certain embodiments, at least 50%, 60%, 70%, 80%, 90%, or 95% of the cells remain viable 60 hours after labeling. In certain embodiments, at least 50%, 60%, 70%, 80%, 90%, or 95% of the cells remain viable 72 hours, or 4 days, 5 days, 6 days, 7 days, 8 days or 9 days after labeling.

Various NIRFs are commercially available and can be used to according to methods of this invention. Exemplary NIRFs include, for example, Cy5, Cy5.5, and Cy7, each of which are available from GE Healthcare; VivoTag-680, VivoTag-S680, VivoTag-S750, each of which are available from VisEn Medical; AlexaFluor660, AlexaFluor680, AlexaFluor700, AlexaFluor750, and Alexa Fluor790, each of which are available from Invitrogen; Dy677, Dy676, Dy682, Dy752, Dy780, each of which are available from Dyonics; DyLight547 and DyLight647, each of which are available from Pierce; HiLyte Fluor 647, HiLyte Fluor 680, and HiLyte Fluor 750, each of which are available from AnaSpec; IRDye800CW, IRDye 800RS, and IRDye 700DX, each of which are available from Li-Cor; and ADS780WS, ADS830WS, and ADS832WS, each of which are available from American Dye Source.

Table 1 lists a number of exemplary fluorochromes useful in the practice of the invention together with their spectral properties.

TABLE 1

| | | Absorbance |
|---|---|---|
| Fluorochrome | εₘₐₓ M⁻¹cm⁻¹ | max (nm) |
| Cy5 | 250,000 | 649 |
| Cy5.5 | 250,000 | 675 |
| Cy7 | 250,000 | 743 |
| AlexaFlour660 | 132,000 | 663 |
| AlexaFlour680 | 184,000 | 679 |
| AlexaFlour700 | 192,000 | 702 |
| AlexaFlour750 | 280,000 | 749 |
| VivoTag-680 (VT680) | 100,000 | 670 |
| VivoTag-S680 | 220,000 | 674 |
| VivoTag-S750 | 100,000 | 750 |
| Dy677 | 180,000 | 673 |
| Dy682 | 140,000 | 690 |
| Dy752 | 270,000 | 748 |
| Dy780 | 170,000 | 782 |
| DyLight547 | 150,000 | 557 |
| DyLight647 | 250,000 | 653 |
| IRDye800CW | 240,000 | 774 |
| IRDye800RS | 200,000 | 767 |
| IRDye700DX | 165,000 | 689 |
| ADS780WS | 170,000 | 782 |
| ADS830WS | 240,000 | 819 |
| ADS832WS | 190,000 | 824 |

In one embodiment, the fluorochrome used to label the cells comprises the molecule of Formula 1:

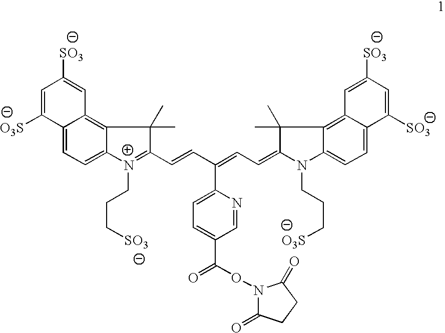

After labeling, the fluorochrome that is covalently linked to the cellular component comprises the molecule of Formula 1′ (the wavy line identifies the covalent linkage between the fluorochrome and the cellular component).

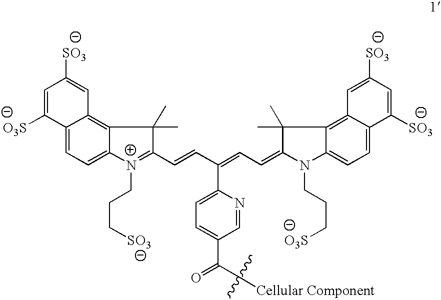

In another embodiment, the fluorochrome used to label the cells comprises the molecule of Formula 2:

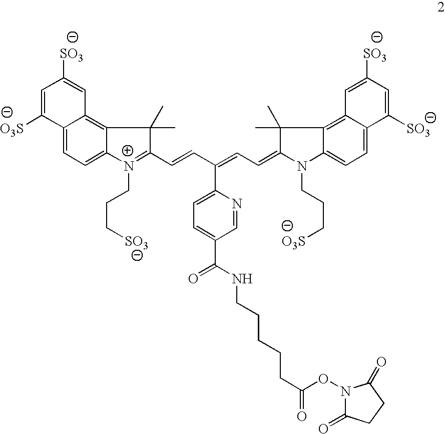

After labeling, the fluorochrome that is covalently linked to the cellular component comprises the molecule of Formula 2′ (the wavy line identifies the covalent linkage between the fluorochrome and the cellular component).

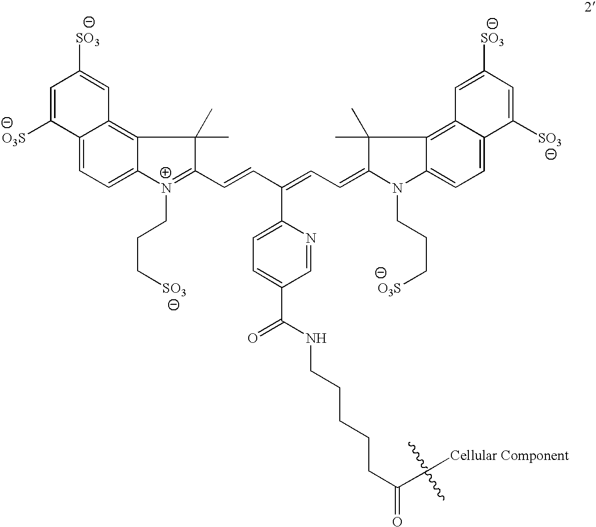

In another embodiment, the fluorochrome used to label the cells comprises the molecule of Formula 3:

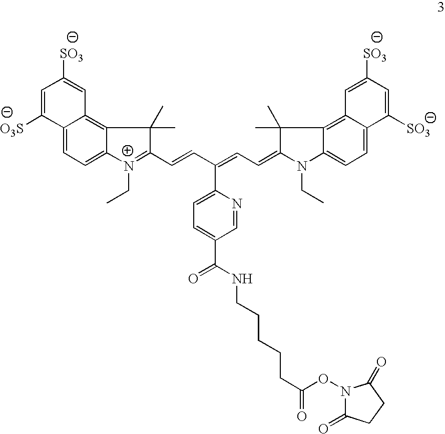

After labeling, the fluorochrome that is covalently linked to the cellular component comprises the molecule of Formula 3′ (the wavy line identifies the covalent linkage between the fluorochrome and the cellular component).

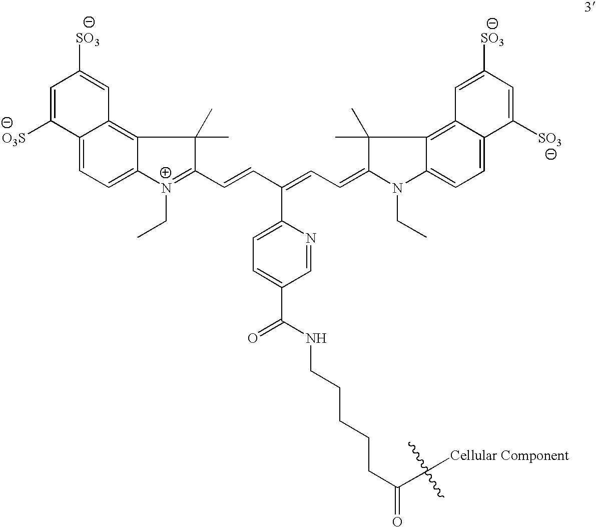

In another embodiment, the fluorochrome used to label the cells comprises the molecule of Formula 4:

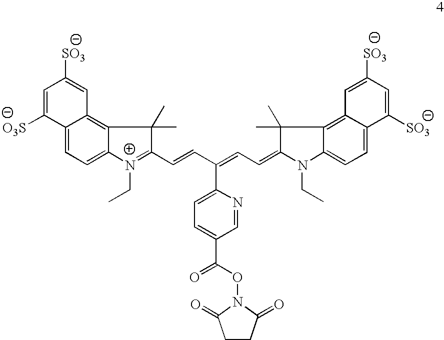

After labeling, the fluorochrome that is covalently linked to the cellular component comprises the molecule of Formula 4′ (the wavy line identifies the covalent linkage between the fluorochrome and the cellular component).

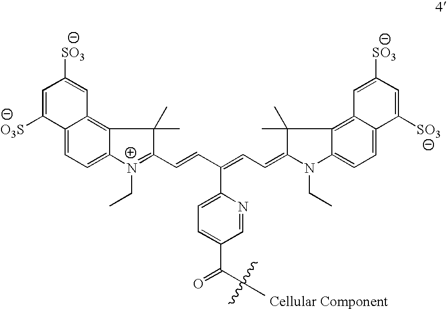

In another embodiment, the fluorochrome used to label the cells comprises the molecule of Formula 5:

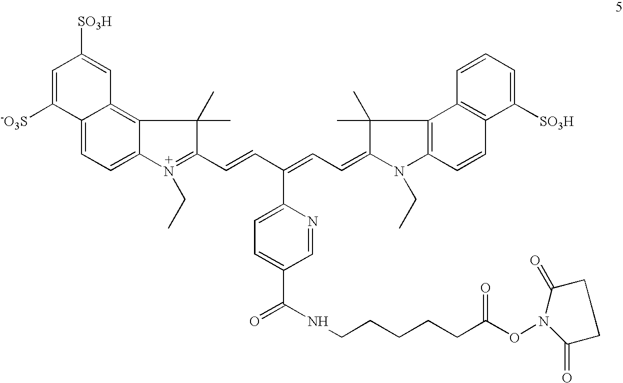

After labeling, the fluorochrome that is covalently linked to the cellular component comprises the molecule of Formula 5′ (the wavy line identifies the covalent linkage between the fluorochrome and the cellular component).

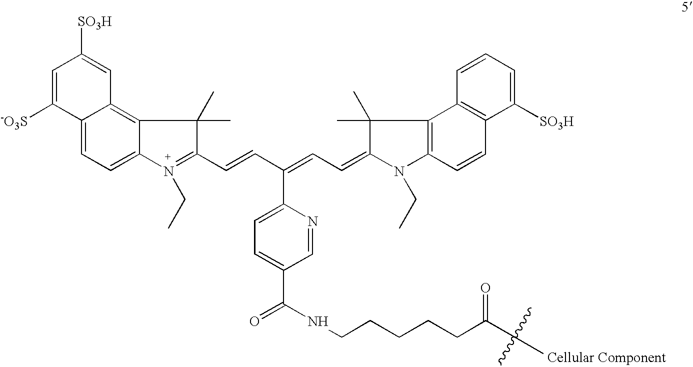

In another embodiment, the fluorochrome used to label the cells comprises the molecule of Formula 6:

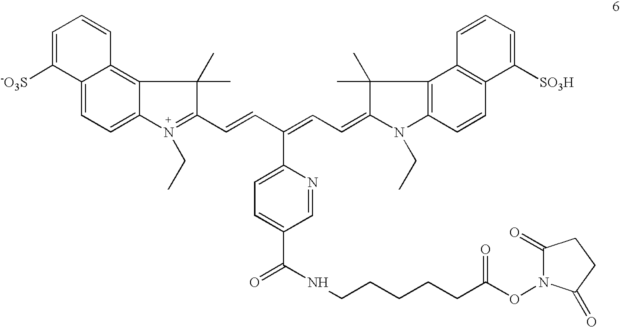

After labeling, the fluorochrome that is covalently linked to the cellular component comprises the molecule of Formula 6′ (the wavy line identifies the covalent linkage between the fluorochrome and the cellular component).

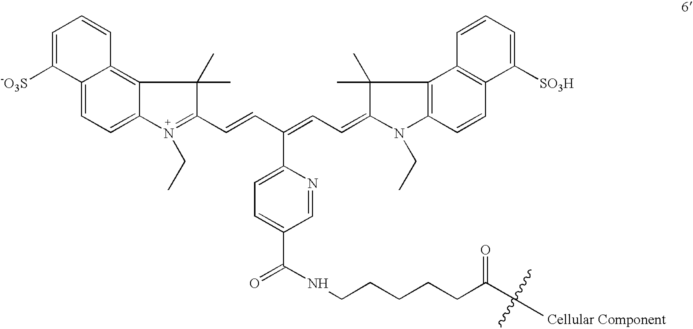

In another embodiment, the fluorochrome used to label the cells comprises the molecule of Formula 7:

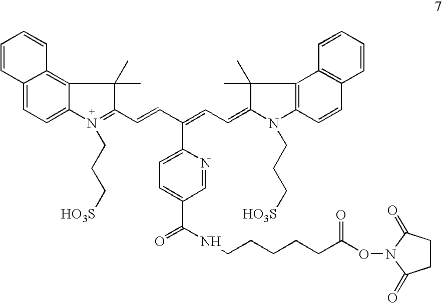

After labeling, the fluorochrome that is covalently linked to the cellular component comprises the molecule of Formula 7′ (the wavy line identifies the covalent linkage between the fluorochrome and the cellular component).

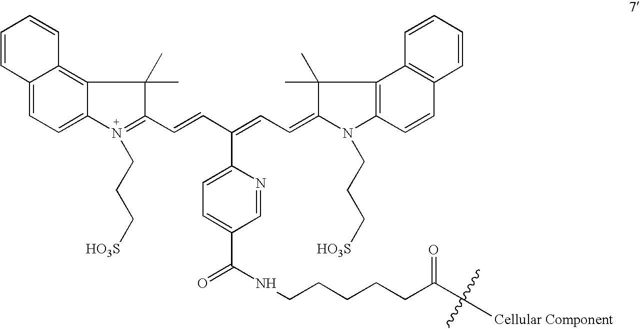

In another embodiment, the fluorochrome used to label the cells comprises the molecule of Formula 8:

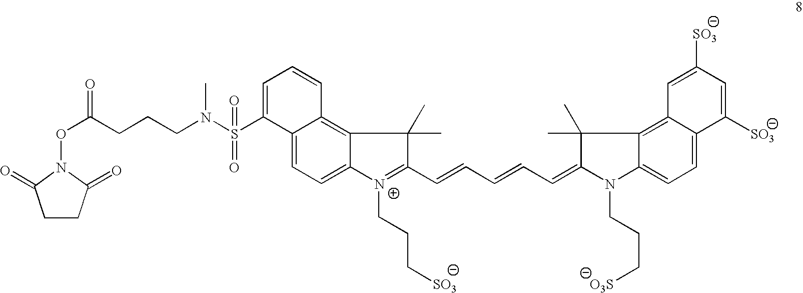

After labeling, the fluorochrome that is covalently linked to the cellular component comprises the molecule of Formula 8′ (the wavy line identifies the covalent linkage between the fluorochrome and the cellular component).

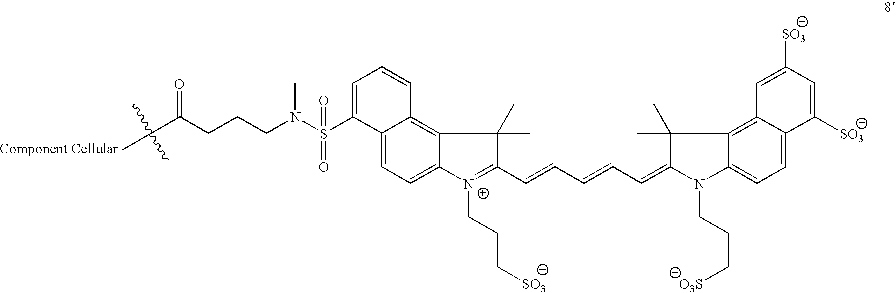

In another embodiment, the fluorochrome used to label the cells comprises the molecule of Formula 9:

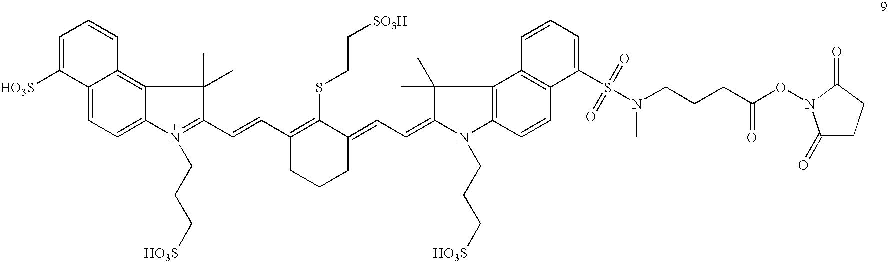

After labeling, the fluorochrome that is covalently linked to the cellular component comprises the molecule of Formula 9′ (the wavy line identifies the covalent linkage between the fluorochrome and the cellular component).

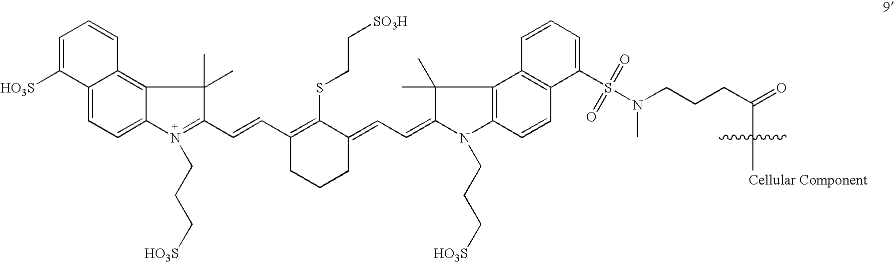

In another embodiment, the fluorochrome used to label the cells comprises the molecule of Formula 10:

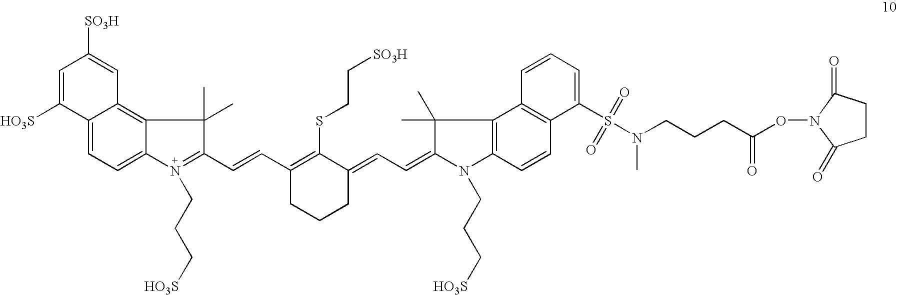

After labeling, the fluorochrome that is covalently linked to the cellular component comprises the molecule of Formula 10′ (the wavy line identifies the covalent linkage between the fluorochrome and the cellular component).

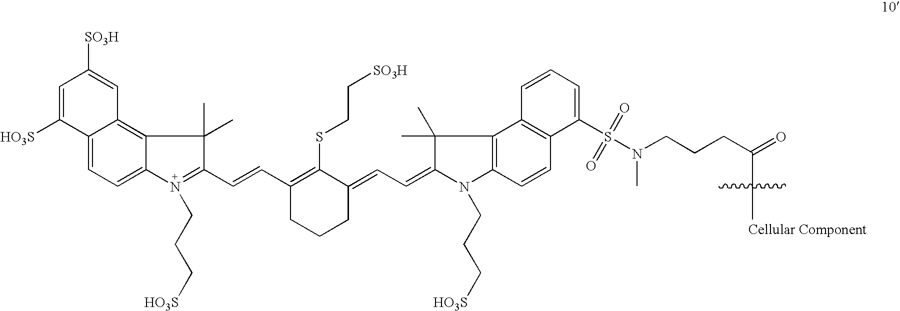

In another embodiment, the fluorochrome used to label the cells comprises the molecule of Formula 11:

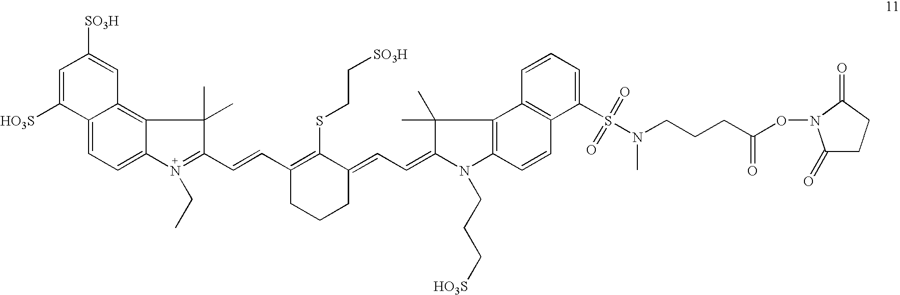

After labeling, the fluorochrome that is covalently linked to the cellular component comprises the molecule of Formula 11′ (the wavy line identifies the covalent linkage between the fluorochrome and the cellular component).

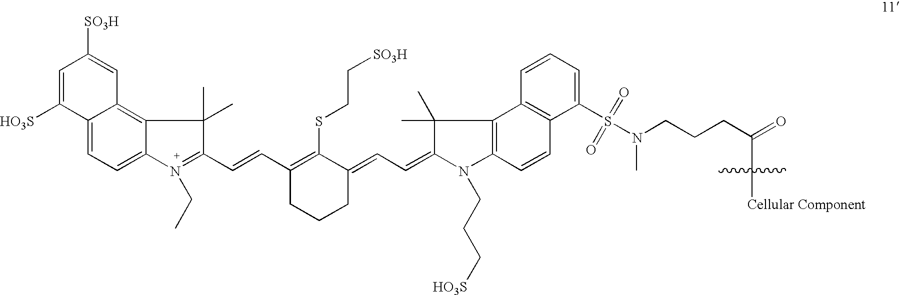

In another embodiment, the fluorochrome used to label the cells comprises the molecule of Formula 12:

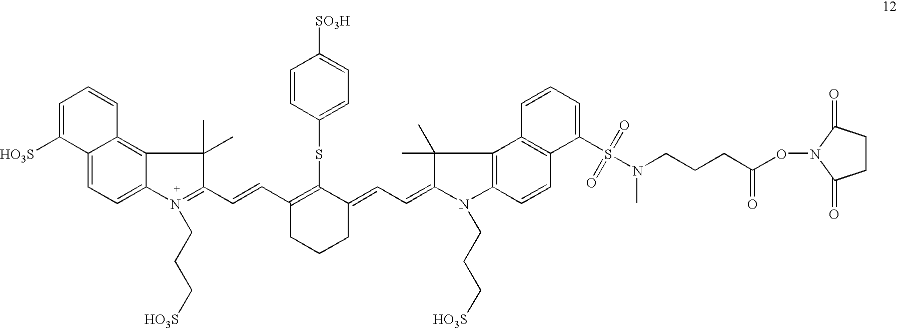

After labeling, the fluorochrome that is covalently linked to the cellular component comprises the molecule of Formula 12′ (the wavy line identifies the covalent linkage between the fluorochrome and the cellular component).

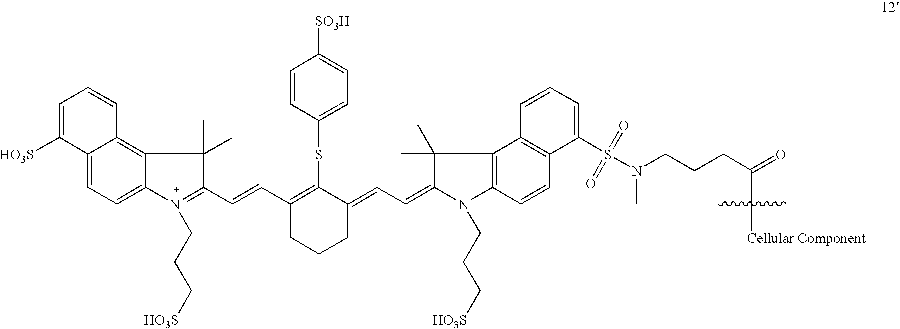

In another embodiment, the fluorochrome used to label the cells comprises the molecule of Formula 13:

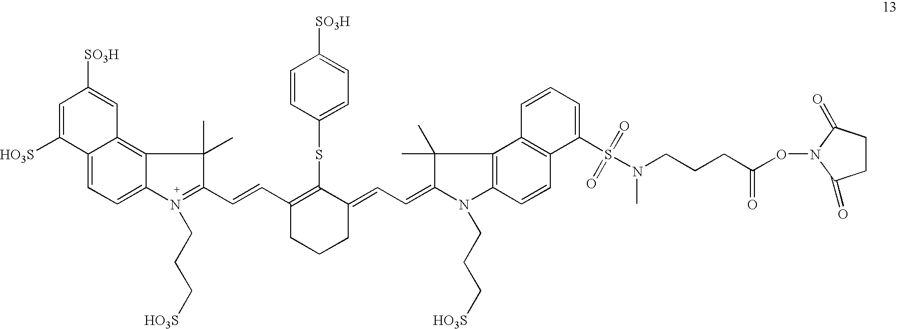

After labeling, the fluorochrome that is covalently linked to the cellular component comprises the molecule of Formula 13′ (the wavy line identifies the covalent linkage between the fluorochrome and the cellular component).

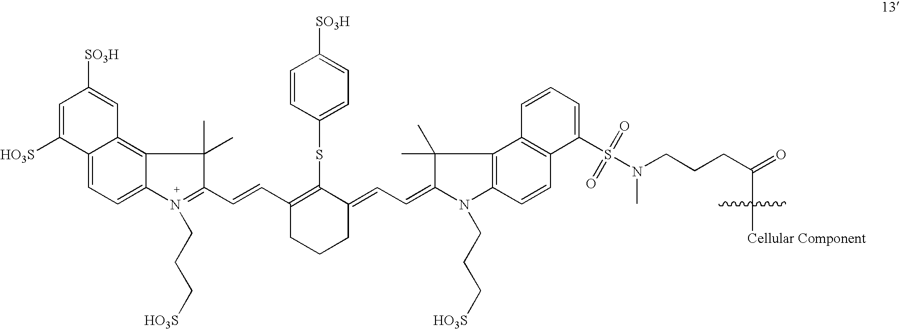

In another embodiment, the fluorochrome used to label the cells comprises the molecule of Formula 14:

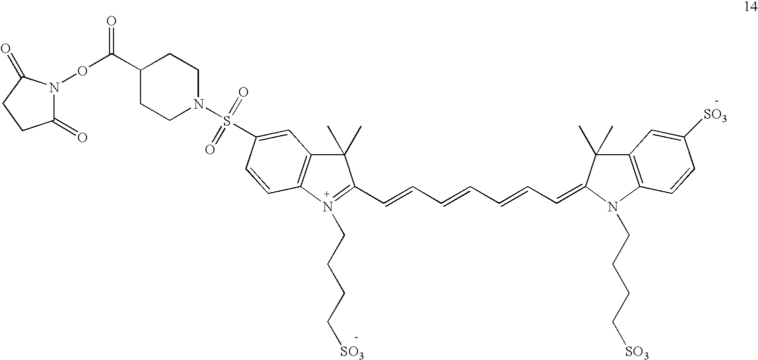

After labeling, the fluorochrome that is covalently linked to the cellular component comprises the molecule of Formula 14′ (the wavy line identifies the covalent linkage between the fluorochrome and the cellular component).

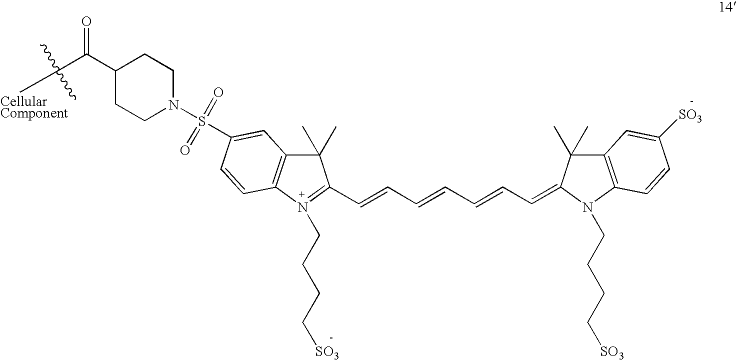

In another embodiment, the fluorochrome used to label the cells comprises the molecule of Formula 15:

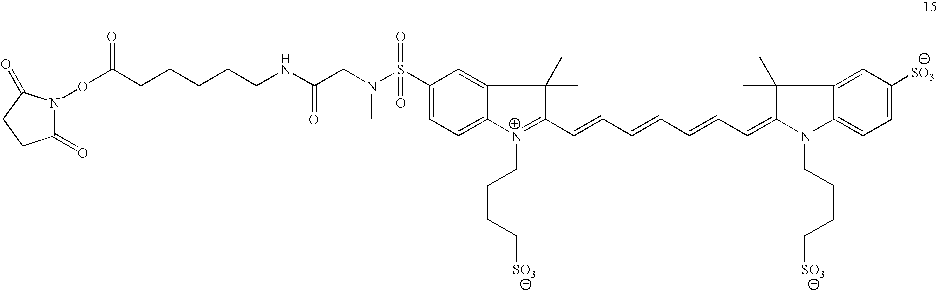

After labeling, the fluorochrome that is covalently linked to the cellular component comprises the molecule of Formula 15′ (the wavy line identifies the covalent linkage between the fluorochrome and the cellular component).

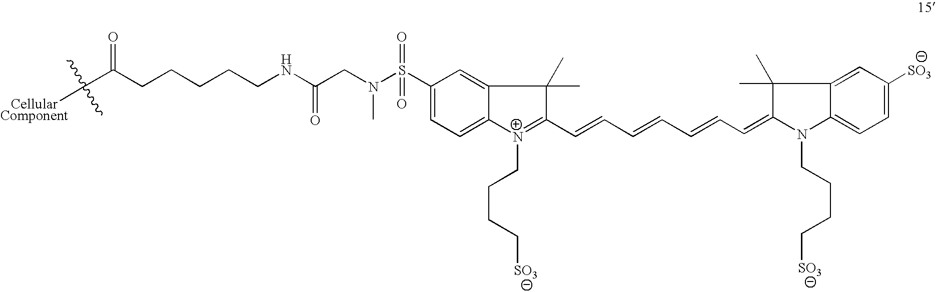

In another embodiment, the fluorochrome used to label the cells comprises the molecule of Formula 16:

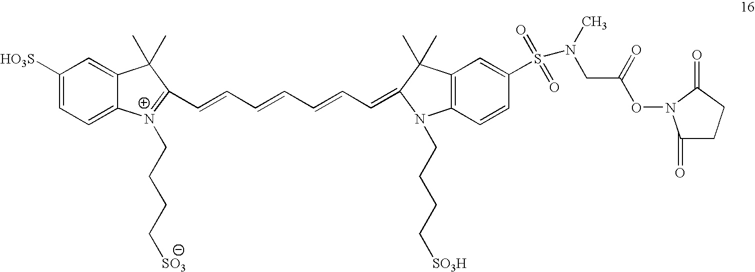

After labeling, the fluorochrome that is covalently linked to the cellular component comprises the molecule of Formula 16′ (the wavy line identifies the covalent linkage between the fluorochrome and the cellular component).

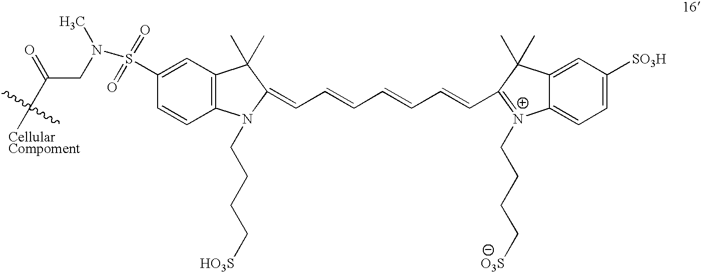

In another embodiment, the fluorochrome used to label the cells comprises the molecule of Formula 17:

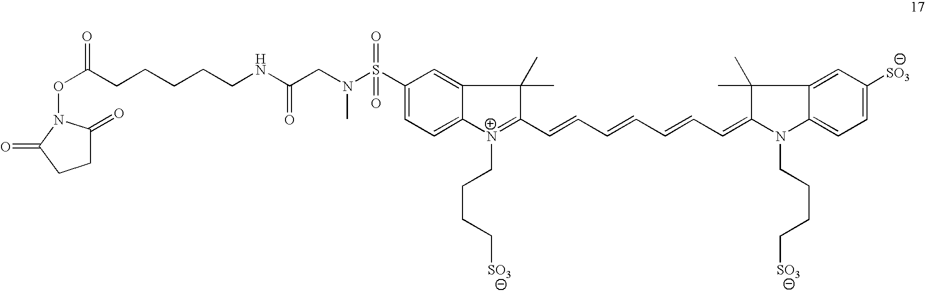

After labeling, the fluorochrome that is covalently linked to the cellular component comprises the molecule of Formula 17′ (the wavy line identifies the covalent linkage between the fluorochrome and the cellular component).

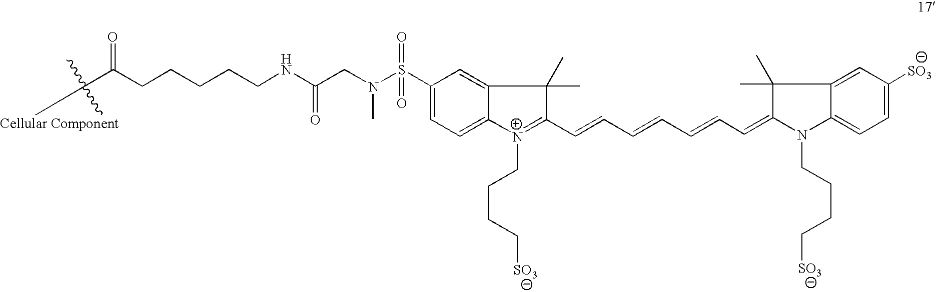

In another embodiment, the fluorochrome used to label the cells comprises the molecule of Formula 18:

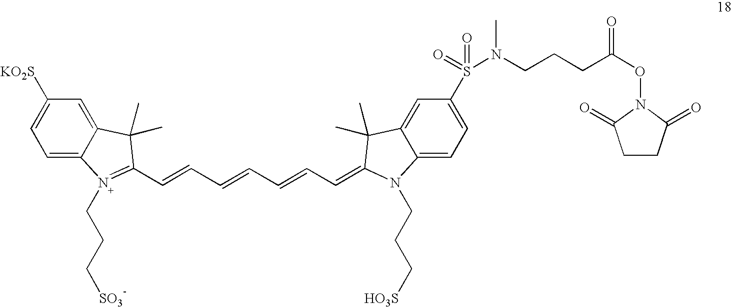

After labeling, the fluorochrome that is covalently linked to the cellular component comprises the molecule of Formula 18′ (the wavy line identifies the covalent linkage between the fluorochrome and the cellular component).

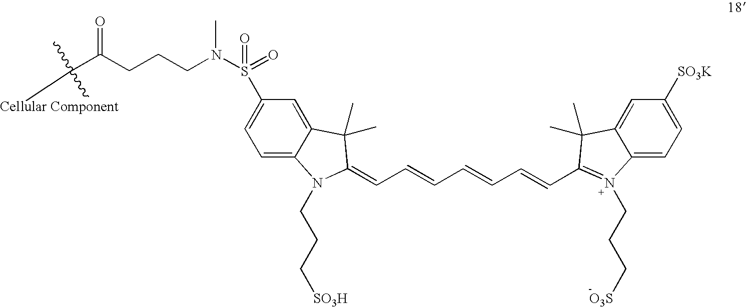

In another embodiment, the fluorochrome used to label the cells comprises the molecule of Formula 19:

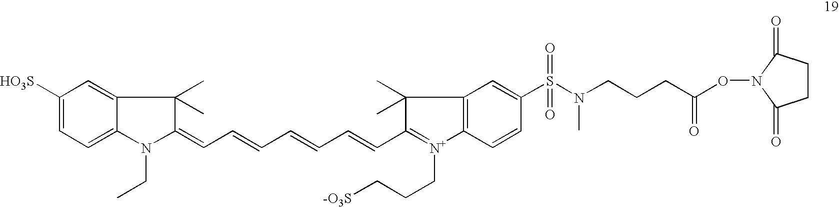

After labeling, the fluorochrome that is covalently linked to the cellular component comprises the molecule of Formula 19′ (the wavy line identifies the covalent linkage between the fluorochrome and the cellular component).

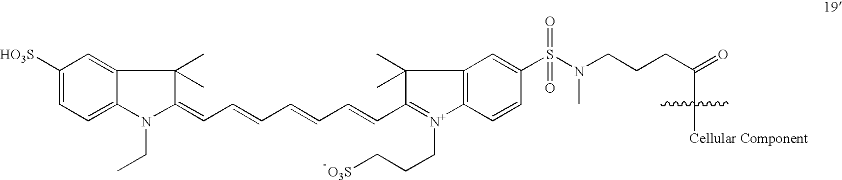

In another embodiment, the fluorochrome used to label the cells comprises the molecule of Formula 20:

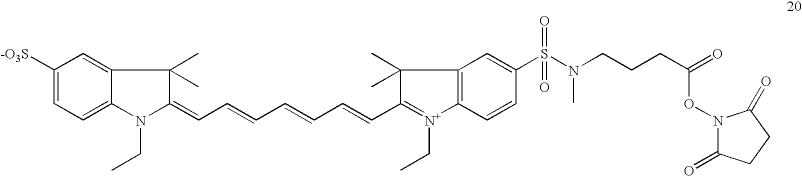

After labeling, the fluorochrome that is covalently linked to the cellular component comprises the molecule of Formula 20′ (the wavy line identifies the covalent linkage between the fluorochrome and the cellular component).

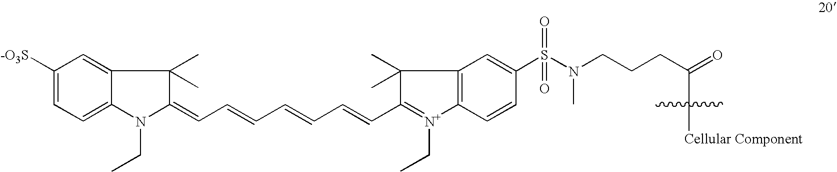

In another embodiment, the fluorochrome used to label the cells comprises the molecule of Formula 21:

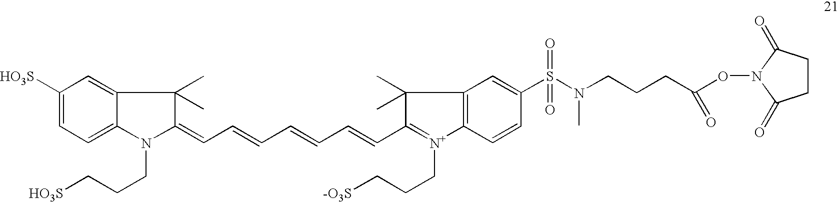

After labeling, the fluorochrome that is covalently linked to the cellular component comprises the molecule of Formula 21′ (the wavy line identifies the covalent linkage between the fluorochrome and the cellular component).

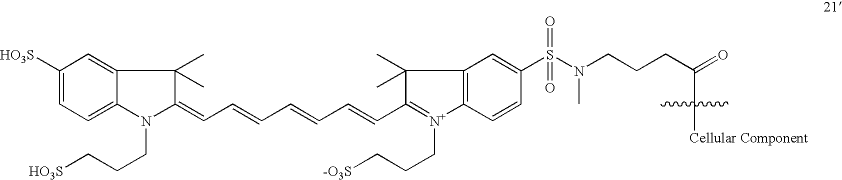

In another embodiment, the fluorochrome used to label the cells comprises the molecule of Formula 22:

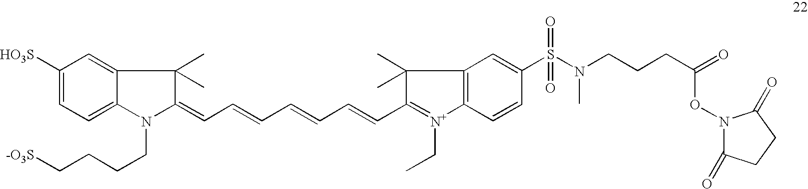

After labeling, the fluorochrome that is covalently linked to the cellular component comprises the molecule of Formula 22′ (the wavy line identifies the covalent linkage between the fluorochrome and the cellular component).

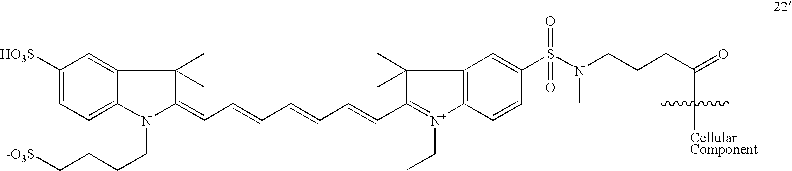

In another embodiment, the fluorochrome used to label the cells comprises the molecule of Formula 23:

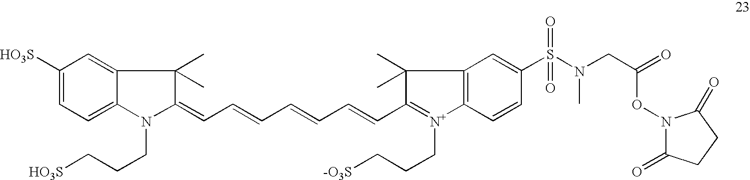

After labeling, the fluorochrome that is covalently linked to the cellular component comprises the molecule of Formula 23′ (the wavy line identifies the covalent linkage between the fluorochrome and the cellular component).

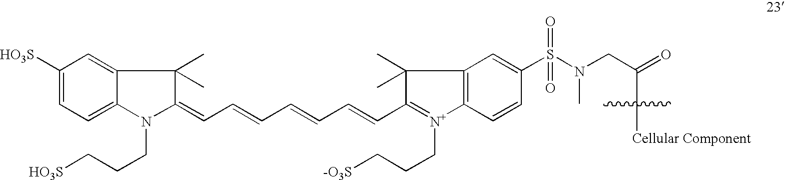

In another embodiment, the fluorochrome used to label the cells comprises the molecule of Formula 24:

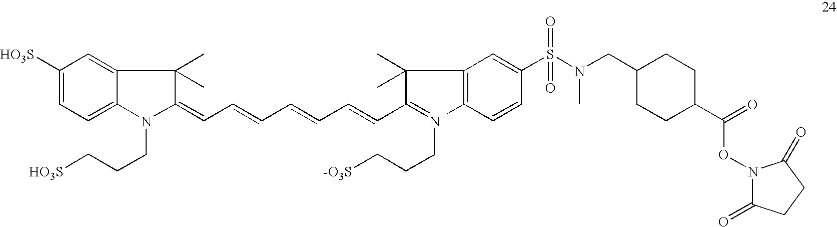

After labeling, the fluorochrome that is covalently linked to the cellular component comprises the molecule of Formula 24′ (the wavy line identifies the covalent linkage between the fluorochrome and the cellular component).

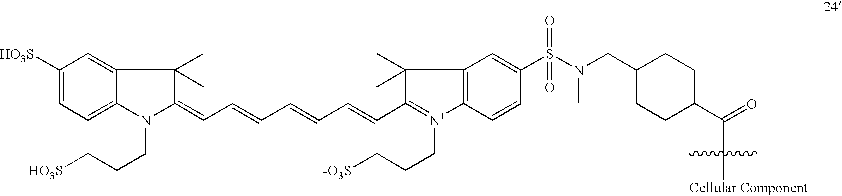

In another embodiment, the fluorochrome used to label the cells comprises the molecule of Formula 25:

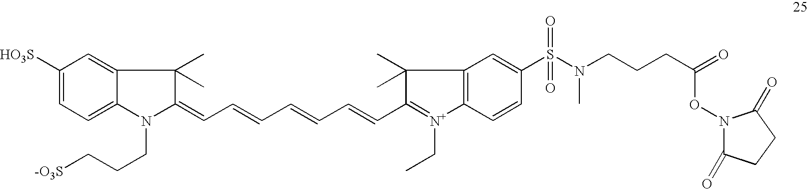

After labeling, the fluorochrome that is covalently linked to the cellular component comprises the molecule of Formula 25′ (the wavy line identifies the covalent linkage between the fluorochrome and the cellular component).

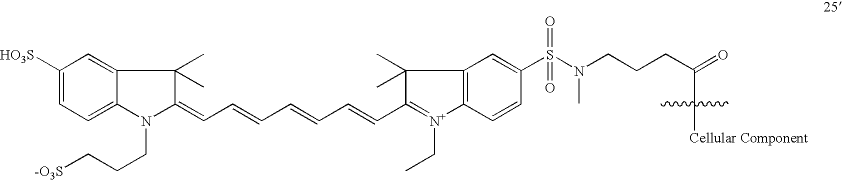

In another embodiment, the fluorochrome used to label the cells comprises the molecule of Formula 26:

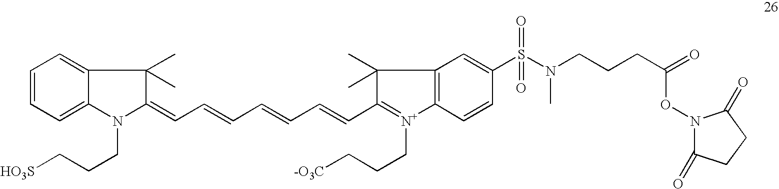

After labeling, the fluorochrome that is covalently linked to the cellular component comprises the molecule of Formula 26′ (the wavy line identifies the covalent linkage between the fluorochrome and the cellular component).

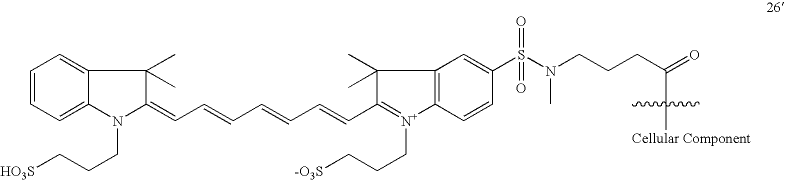

In another embodiment, the fluorochrome used to label the cells comprises the molecule of Formula 27:

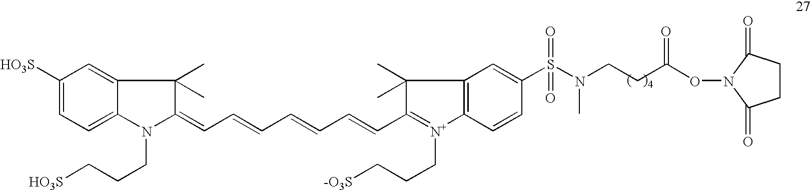

After labeling, the fluorochrome that is covalently linked to the cellular component comprises the molecule of Formula 27′ (the wavy line identifies the covalent linkage between the fluorochrome and the cellular component).

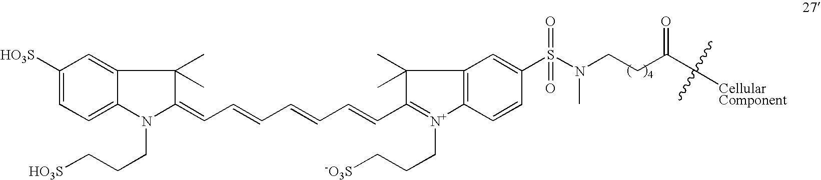

In another embodiment, the fluorochrome used to label the cells comprises the molecule of Formula 28:

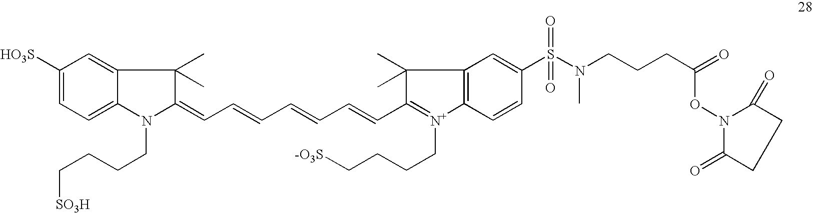

After labeling, the fluorochrome that is covalently linked to the cellular component comprises the molecule of Formula 28′ (the wavy line identifies the covalent linkage between the fluorochrome and the cellular component).

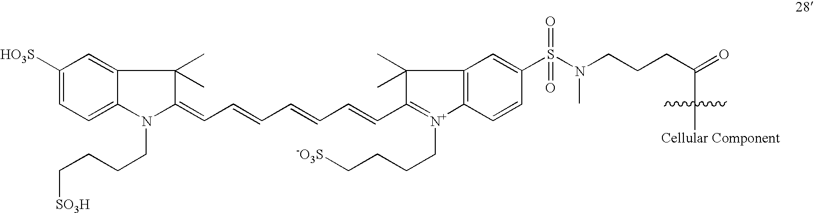

In another embodiment, the fluorochrome used to label the cells comprises the molecule of Formula 29:

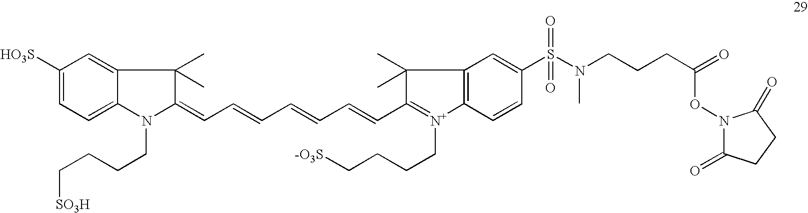

After labeling, the fluorochrome that is covalently linked to the cellular component comprises the molecule of Formula 29′ (the wavy line identifies the covalent linkage between the fluorochrome and the cellular component).

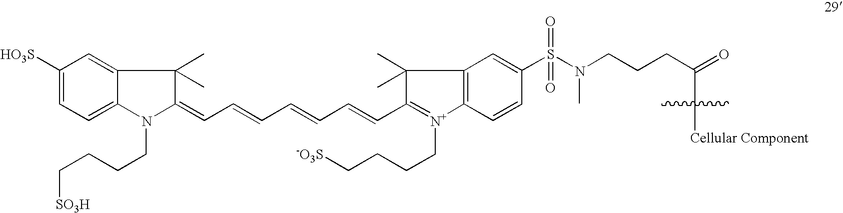

In another embodiment, the fluorochrome used to label the cells comprises the molecule of Formula 30:

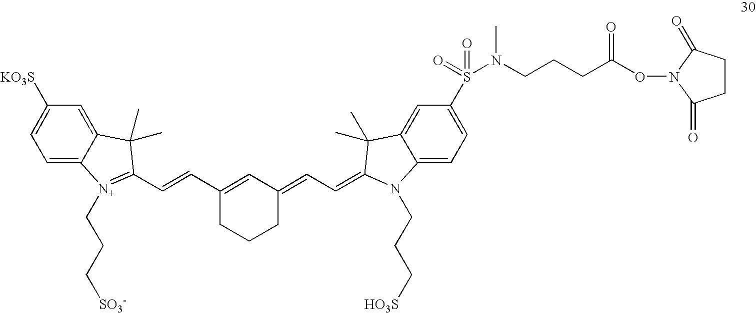

After labeling, the fluorochrome that is covalently linked to the cellular component comprises the molecule of Formula 30′ (the wavy line identifies the covalent linkage between the fluorochrome and the cellular component).

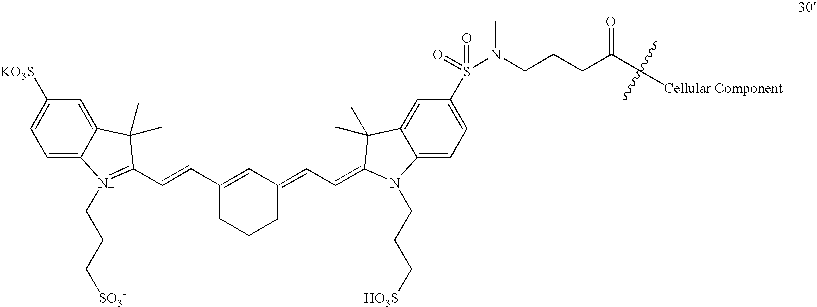

In another embodiment, the fluorochrome used to label the cells comprises the molecule of Formula 31:

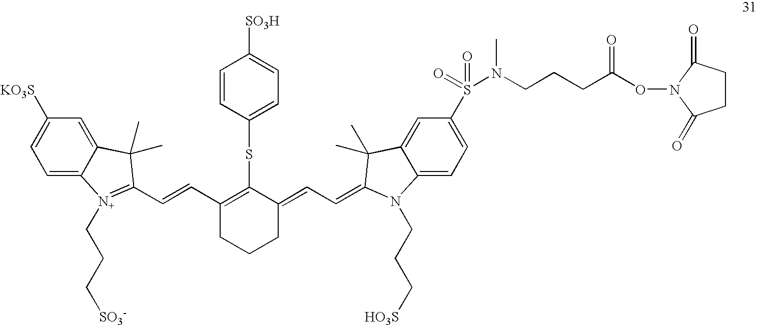

After labeling, the fluorochrome that is covalently linked to the cellular component comprises the molecule of Formula 31′ (the wavy line identifies the covalent linkage between the fluorochrome and the cellular component).

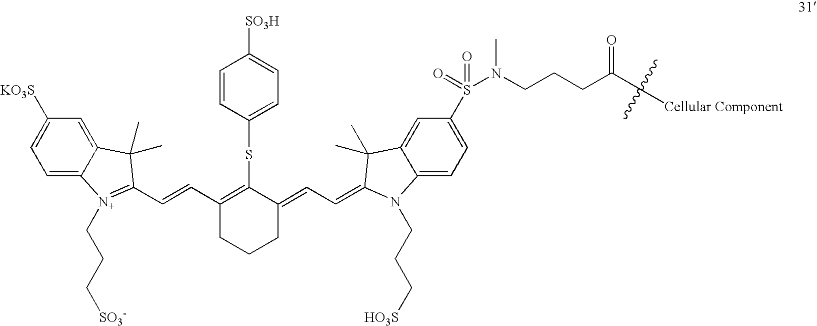

In another embodiment, the fluorochrome used to label the cells comprises the molecule of Formula 32:

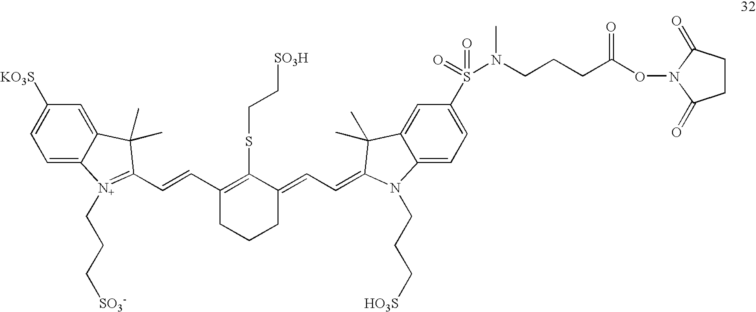

After labeling, the fluorochrome that is covalently linked to the cellular component comprises the molecule of Formula 32′ (the wavy line identifies the covalent linkage between the fluorochrome and the cellular component).

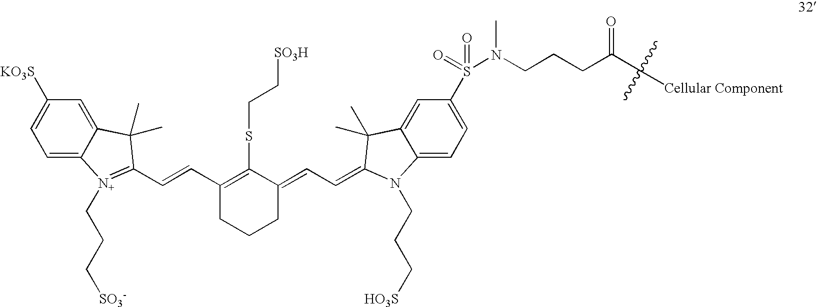

The viable near-infrared fluorochrome labeled cells for use in in vivo imaging are produced as follows. A plurality of viable cells are contacted with a solution comprising near-infrared fluorochrome molecules under conditions that (i) permit at least one near-infrared fluorochrome molecule to become associated (either covalently associated or non covalently associated) to all or a subpopulation of the cells and (ii) maintain the viability of the cells. Under certain circumstances, the cells retain substantially the same viability and/or function as the cells prior to labeling. Under certain circumstances, at least 50%, 60%, 70%, 80%, 90%, or 95% of the cells remain viable after labeling (for, at least, one hour after labeling).

In certain embodiments, the cells are labeled with one or more of fluorochromes of Formula 1-Formula 32, wherein the fluorochrome becomes covalently coupled to a cellular component, for example, as shown in Formula 1′-Formula 32′, respectively.

In certain other embodiments, the near-infrared fluorochrome molecules can be the near infrared fluorochrome of Formula I or II (below).

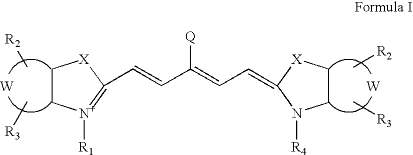

or a salt thereof, wherein:
X is independently selected from the group consisting of C(CH₂Y₁)(CH₂Y₂), O, S, and Se;
Y₁ and Y₂ are independently selected from the group consisting of H, C₁-C₂₀ aliphatic group, and a C₁-C₂₀ aliphatic group substituted with —OR*, N(R*)₂ or —SR*;
W represents a benzo-condensed, a naphtho-condensed or a pyrido-condensed ring;
R₁ is selected from the group consisting of H, (CH₂)ₓCH₃, (CH₂)ₙSO₃⁻ and (CH₂)ₙSO₃H, wherein x is an integer selected from 0 to 6 and n is an integer selected from 2 to 6;
R₂ and R₃ are independently selected from the group consisting of H, carboxylate, carboxylic acid, carboxylic ester, amine, amide, sulfonamide, hydroxyl, alkoxyl, a sulphonic acid moiety and a sulphonate moiety;
R₄ is selected from the group consisting of H, (CH₂)ₓCH₃, (CH₂)ₙSO₃⁻ and (CH₂)SO₃H, wherein x is an integer selected from 0 to 6 and n is an integer selected from 2 to 6; and
Q is selected from a group consisting of a heteroaryl ring substituted with a carboxyl group or 6-membered heteroaryl ring substituted with a carbonyl group.

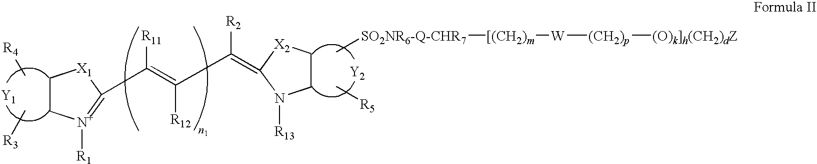

or a salt thereof, wherein:
X₁ and X₂ are independently selected from the group consisting of C(CH₂K₁)(CH₂K₂), O, S and Se;
K₁ and K₂ are independently selected from the group consisting of H, a C₁-C₂₀ aliphatic group and a C₁-C₂₀ aliphatic group substituted with —OR*, N(R*)₂ or —SR*; or K₁ and K₂ together are part of a substituted or unsubstituted carbocyclic or heterocyclic ring;
Y₁ and Y₂ are each independently a benzo-condensed ring, a naphtha-condensed ring or a pyrido-condensed ring;
R₂, R₁₁ and R₁₂ are independently H, halogen, alkyl, alkoxy, aryloxy, aryl, a sulfonate, a group containing SO₂NR₆-Q-CHR₇—(CH₂)ₘ; i is 0 or 1; and m=0-12, an iminium ion, S-aryl, S-alkyl, or any two adjacent R₁₂ and R₁₁ substituents or R₂ and R₁₁ substituents, when taken in combination, form a 4-, 5-, or 6-membered substituted or unsubstituted carbocyclic ring, substituted or unsubstituted non-aromatic carbocyclic ring or a substituted or unsubstituted carbocyclic aryl ring, wherein the carbocyclic rings are each independently optionally substituted one or more times by C₁-C₆ alkyl, halogen, or OR* or SR*;
R₁ and R₁₃ are —H, (CH₂)ₓCH₃, when x is an integer selected from 0 to 6; or R₁ and R₁₃ are independently (CH₂)ₙSO₃⁻ or (CH₂)ₙSO₃H when n is an integer selected from 2 to 6;
R₃, R₄ and R₅ are independently selected from the group consisting of H, carboxylate, carboxylic acid, carboxylic ester, amine, amide, sulfonamide, hydroxyl, alkoxyl, a sulphonic acid moiety and a sulphonate moiety;
R₆ is selected from the group consisting of a substituted or unsubstituted C₁-C₂₀ aliphatic group, a substituted or unsubstituted aryl, a substituted or unsubstituted alkylaryl, wherein R₆ is optionally substituted with halogen, OR*, N(R*)₂ or SR* when Q is absent, a carbonyl group, a substituted or unsubstituted C₁-C₆ alkyl group, wherein 0-2 of the methylene groups of the alkyl group are replaced by NH, O or S, or a substituted or unsubstituted C₁-C₆ carbocyclic, non-aromatic carbocyclic, heterocyclic or non-aromatic heterocyclic ring wherein the heterocyclic rings contains 1-2 heteroatoms; or
R₆ is H, when Q is a carbonyl; and
R₇ is selected from the group consisting of H, a substituted or unsubstituted C₁-C₂₀ aliphatic group, a substituted or unsubstituted aryl, a substituted or unsubstituted alkylaryl, wherein R₇ is optionally substituted with halogen, OR*, N(R*)₂ or SR*; or
R₆ and R₇, taken together form a 4-, 5-, 6- or 7-membered heterocyclic or non-aromatic heterocyclic ring optionally substituted with halogen, OR*, N(R*)₂ or SR*; or
NR₆, Q and CHR₇ together form a substituted or unsubstituted or heterocyclic or non-aromatic heterocyclic ring system wherein the rings contain 1 or 2 heteroatoms, wherein rings are optionally substituted with —OR*, N(R*)₂ or —SR*; and
W is absent or is a group selected from the group consisting of —SO₂NR₆-Q-CHR₇—, —O—, —COO—, and —CONH—;
Z is, or contains a N, O or S nucleophile functionality or is, or contains a functionality capable of reacting with N, O or S nucleophiles;
   h=0-70;
   k=0 or 1;
   d=0-12;
   m=0-12;
   n₁ is 1, 2, or 3;
   p=0-12; and
   each R* is independently —H or C₁₋₂₀ alkyl.

For example, the cells are incubated with various concentrations of a NIRF for about 5 minutes to 24 hours or more at a temperature from about 4° C. to about 37° C. Depending upon the NIRF used, the NIRF can be solubilized in an aqueous rather than an organic solvent, which could be detrimental to the viability of the cells. For example, the cells, in buffer, for example, phosphate buffer saline (PBS) optionally supplemented with bovine serum albumin (BSA), are incubated with the fluorochrome (at a final concentration of 5-50 μg/mL) on ice, for example, 5 minutes to 10 hours, with periodic agitation, for example, every 5 minutes. Although less desirable, the fluorochromes can be reconstituted in an organic solvent, for example, dimethyl sulfoxide (DMSO) and then added to the cells. Aqueous solvents, however, generally are preferred so as to preserve the viability of the cells.

After incubation, unbound NIRF can be removed using methods known to those skilled in art, for example, by washing, chromatography or ultrafiltration. For example, the cells can be centrifuged after incubation to create a cell pellet from which the supernatant is removed. Cells then are resuspended in culture media or physiologic saline (for example, in PBS optionally supplemented with 0.5% bovine serum albumin (BSA)) to wash away residual, unbound NIRF. This can be repeated several times. In this manner, cells can be labeled by conjugation (through a covalent linkage or adsorption) to internal or external cellular components.

The resulting cells can be used immediately or after storage on ice in a storage medium comprising a supplemental media suitable for the health and viability of the cells. The cells can be administered locally or systemically using techniques known in the art. Following administration the labeled cells can be detected using imaging systems known in the art. An imaging system useful in the practice of this invention typically includes three basic components: (1) an appropriate light source for exciting the fluorochrome labeled cells of the invention, (2) a system for separating or distinguishing emissions from light used for inducing fluorochrome excitation, and (3) a detection system. This detection system can be hand-held or incorporated into other useful imaging devices such as endoscopes, catheters, intraoperative microscopes and/or viewers.

Preferably, the light source provides monochromatic (or substantially monochromatic) light. The light source can be a suitably filtered white light, i.e., bandpass light from a broadband source. For example, light from a 150-watt halogen lamp can be passed through a suitable bandpass filter commercially available from Omega Optical (Brattleboro, Vt.). Depending upon the system, the light source can be a laser. See, e.g., Boas et al., Proc. Natl. Acad. Sci. USA 91:4887-4891, 1994; Ntziachristos et al., Proc. Natl. Acad. Sci. USA 97:2767-2772, 2000; and Alexander, J. Clin. Laser Med. Surg. 9:416-418, 1991. Information on lasers useful in in vivo imaging can be found, for example, at Imaging Diagnostic Systems, Inc., Plantation, Fla. and various other sources. A high pass or bandpass filter can be used to separate optical emissions from excitation light. A suitable high pass or bandpass filter is commercially available from Omega Optical, Burlington, Vt.

In general, the light detection system can be viewed as including a light gathering/image forming component and a light detection/image recording component. Although the light detection system can be a single integrated device that incorporates both components, the light gathering/image forming component and light detection/image recording component are discussed separately.

A particularly useful light gathering/image forming component is an endoscope. Endoscopic devices and techniques which have been used for in vivo optical imaging of numerous tissues and organs, including peritoneum (Gahlen et al., J. Photochem. Photobiol. B 52:131-135, 1999), ovarian cancer (Major et al., Gynecol. Oncol. 66:122-132, 1997), colon and rectum (Mycek et al., Gastrointest. Endosc. 48:390-394, 1998; and Stepp et al., Endoscopy 30:379-386, 1998), bile ducts (Izuishi et al., Hepatogastroenterology 46:804-807, 1999), stomach (Abe et al., Endoscopy 32:281-286, 2000), bladder (Kriegmair et al., Urol. Int. 63:27-31, 1999; and Riedl et al., J. Endourol. 13:755-759, 1999), lung (Hirsch et al., Clin Cancer Res 7:5-220, 2001), brain (Ward, J. Laser Appl. 10:224-228, 1998), esophagus, and head and neck regions can be employed in the practice of the present invention.

Other types of light gathering components are catheter-based devices, including fiber optics devices. Such devices are particularly suitable for intravascular imaging. See, for example, Tearney et al., Science 276: 2037-2039, 1997; and Circulation 94: 3013, 1996.

Still other imaging technologies, including phased array technology (Boas et al., Proc. Natl. Acad. Sci. USA 91:4887-4891, 1994; Chance, Ann. NY Acad. Sci. 838:29-45, 1998), optical tomography (Cheng et al., Optics Express 3:118-123, 1998; and Siegel et al., Optics Express 4:287-298, 1999), intravital microscopy (Dellian et al., Br. J. Cancer 82:1513-1518, 2000; Monsky et al, Cancer Res. 59:4129-4135, 1999; and Fukumura et al., Cell 94:715-725, 1998), confocal imaging (Korlach et al., Proc. Natl. Acad. Sci. USA 96:8461-8466, 1999; Rajadhyaksha et al., J. Invest. Dermatol. 104:946-952, 1995; and Gonzalez et al., J. Med. 30:337-356, 1999) and fluorescence molecular tomography (FMT) (Nziachristos et al., Nature Medicine 8:757-760, 2002; U.S. Pat. No. 6,615,063, PCT Application No. WO 03/102558, and PCT US/03/07579) can be used with the fluorochrome compounds of the invention. Similarly, the agents can be used in a variety of imaging systems, for example, the IVIS® Imaging Systems: 100 Series, 200 Series; SPECTRUM and LUMINA (Xenogen, Alameda, Calif.—part of Caliper LifeSciences); SoftScan® or the eXplore Optix™ (GE Healthcare, United Kingdom); Maestro and Nuance-2 Systems (CRi, Woburn, Mass.); Image Station In-Vivo FX from Carestream Molecular Imaging, Rochester, N.Y. (formerly Kodak Molecular Imaging Systems); OV100, IV100 (Olympus Corporation, Japan); Cellvizio Mauna Kea Technologies, France); NanoSPECT/CT or HiSPECT (Bioscan, Washington, D.C.); CTLM or LILA (Imaging Diagnostic Systems, Plantation, Fla.); DYNOT (NIRx Medical Technologies, Glen Head, N.Y.); and NightOWL Imaging Systems by Berthold Technologies, Germany.

A variety of light detection/image recording components, e.g., charge coupled device (CCD) systems or photographic film, can be used in such systems. The choice of light detection/image recording depends on factors including the type of light gathering/image forming component being used. It is understood, however, that the selection of suitable components, the assembly of the components into an optical imaging system, and the operation of the system is within the level of skill in the art.

Fluorescence and optical imaging and measurement techniques include, but are not limited to, fluorescence imaging, luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; intravital imaging; two photon imaging; interferometry; coherence interferometry; diffuse optical tomography and fluorescence molecular tomography.

In addition, the methods of the present invention can be used in combination with other imaging compositions and methods. For example, in addition to fluorescent imaging, the viable cells can be detected by other imaging modalities, such as, X-ray, computed tomography (CT), MR imaging, ultrasound, positron emission tomography (PET), and single photon computerized tomography (SPECT), including co-registration of images. As a result, the image representation of the subject or region within the subject obtained by fluorescent imaging can be co-registered with an image of the subject or the region within the subject obtained by X-ray, CT, MR imaging, PET, and SPECT.

In certain embodiments, the labeled cells are detected within a vertebrate, for example, a mammal, for example, a human, laboratory animals, for example, rats, mice, dogs and farm animals. It is understood, however, that the cells can also be detected within a non-vertebrate (e.g., C. elegans, drosophila, zebra fish or other animal models used in research).

The methods described herein can be used to determine a number of indicia, including tracking the localization of the cells in the subject over time or assessing changes or alterations in the cells in the subject over time. The methods can also be used to follow therapy for such diseases by imaging molecular events and biological pathways modulated by such therapy, including but not limited to determining efficacy, optimal timing, optimal dosing levels (including for individual patients or test subjects), and synergistic effects of combination therapies.

The methods and compositions described herein can also be used to help a physician or surgeon to identify and characterize areas of disease, such as arthritis, cancers and specifically colon polyps, or vulnerable or unstable plaque, to distinguish diseased and normal tissue, such as detecting tumor margins that are difficult to detect using an ordinary operating microscope, e.g., in brain surgery, to help dictate a therapeutic or surgical intervention, for example, by determining whether a lesion is cancerous and should be removed or non-cancerous and left alone, or in surgically staging a disease, for example, intraoperative lymph node staging, sentinel lymph node mapping, or assessing intraoperative bleeding or to delineate tumor margins.

The methods and compositions of the invention can also be used in the detection, characterization and/or determination of the localization of a disease, especially early disease, the severity of a disease or a disease-associated condition, the staging of a disease, and/or monitoring a disease. The presence, absence, or level of an emitted signal can be indicative of a disease state. The methods and compositions of the invention can also be used to monitor and/or guide various therapeutic interventions, such as surgical procedures, and monitoring drug therapy, including cell based therapies. The methods of the invention can also be used in prognosis of a disease or disease condition.

Examples of disease or disease conditions that can be detected or monitored (before, during or after therapy) using the procedures described herein include inflammation (for example, inflammation caused by arthritis, for example, rheumatoid arthritis), cancer (for example, colorectal, ovarian, lung, breast, prostate, cervical, testicular, skin, brain, gastrointestinal, pancreatic, liver, kidney, bladder, stomach, leukemia, mouth, esophageal, bone), cardiovascular disease (for example, atherosclerosis and inflammatory conditions of blood vessels, ischemia, stroke, thrombosis, disseminated intravascular coagulation), dermatologic disease (for example, Kaposi's Sarcoma, psoriasis, allergic dermatitis), ophthalmic disease (for example, macular degeneration, diabetic retinopathy), infectious disease (for example, bacterial, viral, fungal and parasitic infections, including Acquired Immunodeficiency Syndrome, Malaria, Chagas Disease, Schistosomiasis), immunologic disease (for example, an autoimmune disorder, lymphoma, multiple sclerosis, rheumatoid arthritis, diabetes mellitus, lupus erythematosis, myasthenia gravis, Graves disease), central nervous system disease (for example, a neurodegenerative disease, such as Parkinson's disease or Alzheimer's disease, Huntington's Disease, amyotrophic lateral sclerosis, prion disease), inherited diseases, metabolic diseases, environmental diseases (for example, lead, mercury and radioactive poisoning, skin cancer), bone-related disease (for example, osteoporosis, primary and metastatic bone tumors, osteoarthritis), neurodegenerative disease, and surgery-related complications (such as graft rejection, organ rejection, alterations in wound healing, fibrosis or other complications related to surgical implants).

The methods and compositions of the invention, therefore, can be used, for example, to determine the presence and/or localization of tumor cells, the presence and/or localization of inflammation, including the presence of activated macrophages, for instance in atherosclerosis or arthritis, the presence and in localization of vascular disease including areas at risk for acute occlusion (i.e., vulnerable plaques) in coronary and peripheral arteries, regions of expanding aneurysms, unstable plaque in carotid arteries, and ischemic areas. The disclosed methods of the invention can be used, for example, in identification and evaluation of apoptosis, necrosis, hypoxia and angiogenesis. Alternatively, the disclosed methods may also be used to assess the effect of a therapeutic compound or therapy on a specified molecular target by, for example, imaging a subject prior to and after treatment with the therapeutic compound or therapy, and comparing corresponding images.

Throughout the description, where compositions are described as having, including, or comprising specific components, it is contemplated that compositions also consist essentially of, or consist of, the recited components. Similarly, where processes are described as having, including, or comprising specific process steps, the processes also consist essentially of, or consist of, the recited processing steps. Further, it should be understood that the order of steps or order for performing certain actions are immaterial so long as the invention remains operable. Moreover, two or more steps or actions may be conducted simultaneously.

The invention will now be illustrated by means of the following examples, which are given for the purpose of illustration only and without any intention to limit the scope of the present invention.

### EXAMPLES

### Example 1

### Cell Labeling

Mouse splenocytes from 12 week old BALB/c mice (Charles River Laboratories, Wilmington, Mass.) are prepared as a single cell suspension, and the T cell subpopulation within the splenocyte preparation are enriched by passage over a column that can remove B cells and macrophages (R & D kit, Mouse T-cell enrichment columns, MTCC500). T cells are centrifuged to produce a cell pellet of about 10⁷ cells. The supernatant then is removed from the cell pellet. The pellet is resuspended in complete media for several cycles of rinsing and recentrifugation before being resuspended in a final complete media suitable to cell culture with a solution of 10 mg/mL of a near-infrared fluorochrome molecule disclosed herein is added. Cells then are incubated at room temperature for 5 minutes, followed by 2 rounds of centrifugation and resuspension in physiologic buffer to wash away unbound fluorochrome molecules. Cells then are assessed by fluorescence microscopy.

### Example 2

### Cell Labeling and In Vivo Imaging

Mouse 4T1 breast adenocarcinoma cells are centrifuged to generate a cell pellet of about 10⁷ cells. The supernatant is removed from the cell pellet, and a solution of 10 mg/mL of a near-infrared fluorochrome molecule disclosed herein is added. Cells then are incubated at room temperature for 5 minutes, followed by 2 rounds of centrifugation and resuspension in physiological buffer to remove unbound fluorophore. Cells then are be assessed by fluorescence microscopy. Cells then are injected intravenously into mice at 5×10⁵ cells per mouse, and the live mice are imaged by fluorescent molecular tomography immediately after injection and 24 hours after injection. Because 4T1 cells primarily metastasize to the lungs, it is contemplated that lung fluorescence can be quantified.

### Example 3

### Cell Labeling Efficiency Screen

This example describes that it is possible to label viable cells with a variety of fluorochromes including the succinimidyl ester of the fluorochrome of Cy5.5, Formula 3, Formula 4, and VivoTag-680.

One million HT-29 cells in PBS were added to each well of a 96-well tissue culture plate. The fluorophores were reconstituted in DMSO at 1 mg/mL and added to designated wells at 30 μg/mL. The cells then were incubated with fluorophore on ice for 30 minutes with agitation every 5 minutes. The cells then were washed with PBS/0.5% FBS to remove excess fluorophore, and a sample removed from each group for microscopic evaluation. The resulting microscopic images, each of which was gated at the same maximum fluorescence, demonstrate that the cells were labeled effectively with each fluorophore.

### Example 4

### Labeling of Splenocytes with VivoTag-680

Splenocytes contain mixtures of T-cells and B-cells, along with other cell types. Four million splenocytes (depleted of red blood cells) per mL were resuspended in PBS. Fluorophore VivoTag-680 (succinimidyl ester) from VisEn Medical, Bedford Mass. was reconstituted in DMSO at 10 mg/mL and added to cells at 30 μg/mL. The cells were incubated on ice for 20 minutes and then washed with PBS/0.5% BSA to remove excess fluorophore. A sample was taken for microscopic evaluation, which demonstrated that splenocytes can be effectively labeled with the fluorochrome VivoTag-680.

### Example 5

### In Vivo Imaging of Labelled HT-29 Cells

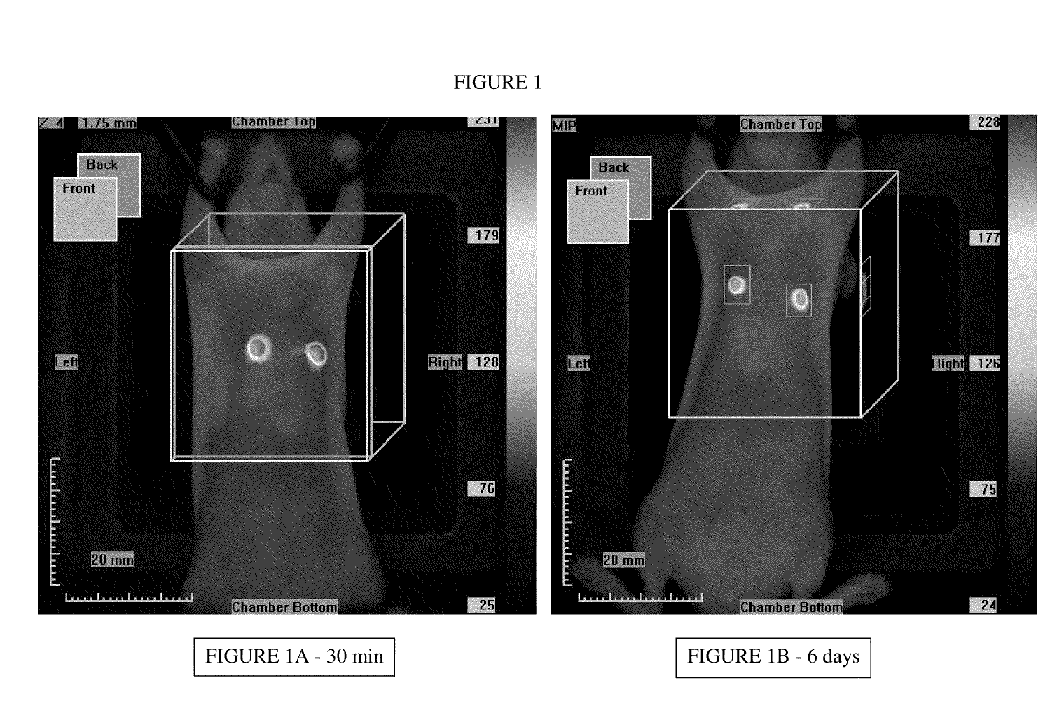

Four million HT-29 cells per mL were resuspended in PBS/0.5% BSA. VivoTag680 (succinimidyl ester) from VisEn Medical, Bedford Mass. was reconstituted in DMSO and added to the cells to give a final concentration of 30 μs/mL. The cells were incubated with VivoTag-680 on ice for 20 minutes, and then washed with PBS/0.5% BSA to remove excess VivoTag-680. Three and a half million labeled cells in 100 μL were injected subcutaneously per site of mammary fat pad of a 6 week old female Nu/Nu mouse (Charles River Laboratories, Wilmington, Mass.). Mice were imaged for colorectal xenograft tumors in the mammary fat pad tissues using the FMT system (VisEn Medical, Bedford, Mass.) starting at 30 minutes. Images of the mouse at 30 minutes and at 6 days are shown in FIGS. 1A and 1B, respectively.

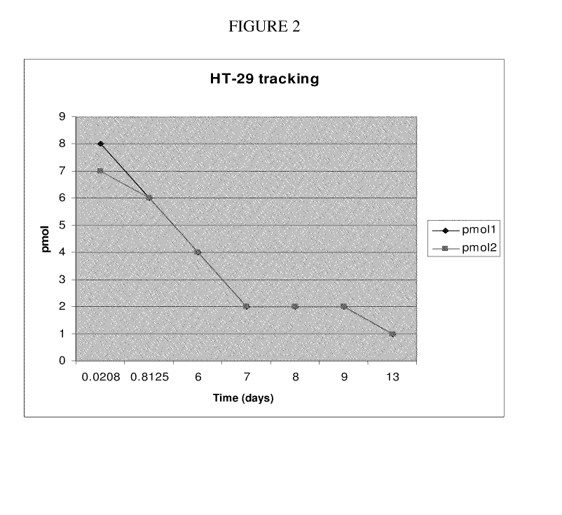

The decrease in fluorescent signal in the two separate tumors shown in FIG. 1 was measured and the results shown in FIG. 2. Fluorescent information was used to assess the volume of the tumor mass. It was found that, despite reduction in fluorescent signal over time, the volume of the tumor could be accurately measured on the thirteenth day.

### Example 6

### Labeling of HT-29 Cells Without DMSO as a Solvent for Fluorochromes

The example demonstrates that it is possible to effectively label viable cells when the fluorochrome is not first dissolved in an organic solvent, for example, DMSO.

One million HT-29 cells in 250 μL PBS were placed in wells of a microtiter plate. Then 10 μg/mL final solutions of the fluorophores, Cy5.5 (succinimidyl ester) and Formula 3 (succinimidyl ester) in PBS were added to each well. The cells were incubated with fluorophore on ice for 1.5 hours with agitation every 15 minutes, and then were washed with PBS/0.5% BSA to remove excess fluorophore. A sample was taken for microscopic evaluation. The results demonstrated that the HT 29 cells were effectively labeled with both the Cy5.5 fluorochrome and the fluorochrome of Formula 3.

### INCORPORATION BY REFERENCE

All publications, patents, and patent applications cited herein and listed below are hereby expressly incorporated by reference in their entirety and for all purposes to the same extent as if each was so individually denoted.

### EQUIVALENTS

The invention may be embodied in other specific forms without departing form the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. An in vivo imaging method for tracking and/or locating and/or determining a quantity of viable cells in a subject, the method comprising the steps of:
a) administering to the subject a plurality of viable cells covalently labeled with a near-infrared fluorochrome, wherein at least 50% of the cells remain viable after labeling as determined by a Trypan Blue exclusion assay;
b) directing near-infrared excitation light into the subject; and
c) detecting fluorescent light emitted from the cells thereby to track and/or locate and/or determine the quantity of the cells in the subject.

2. The method of claim 1, further comprising the step of, after step c), processing the detected fluorescent light emitted from the cells to create an image representation of the subject or a region within the subject.

3. The method of claim 2, wherein the image representation is a tomographic image.

4. (canceled)

5. The method of claim 1, further comprising repeating steps b) and c) at discrete or continuous points in time.

6. The method of claim 1, wherein step (a) comprises administering the cells systemically.

7. The method of claim 1, wherein step (a) comprises administering the cells locally.

8. The method of claim 1, wherein the subject is a mammal.

9. The method of claim 1, wherein the subject is a human.

10. The method of claim 1, wherein the near-infrared fluorochrome is a carbocyanine dye.

11. (canceled)

12. The method of claim 1, wherein the near-infrared fluorochrome is selected from the group consisting of Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, and DyLight647, HiLyte Fluor 680, HiLyte Fluor 750, IRDye800CW, IRDye 800RS, IRDDye 700DX, ADS780WS, and ADS832WS.

13. The method of claim 1, wherein the near-infrared fluorochrome is covalently linked to the cell through a chemically reactive functional group.

14. (canceled)

15. The method of claim 1, wherein the near-infrared fluorochrome is selected from the group consisting of:

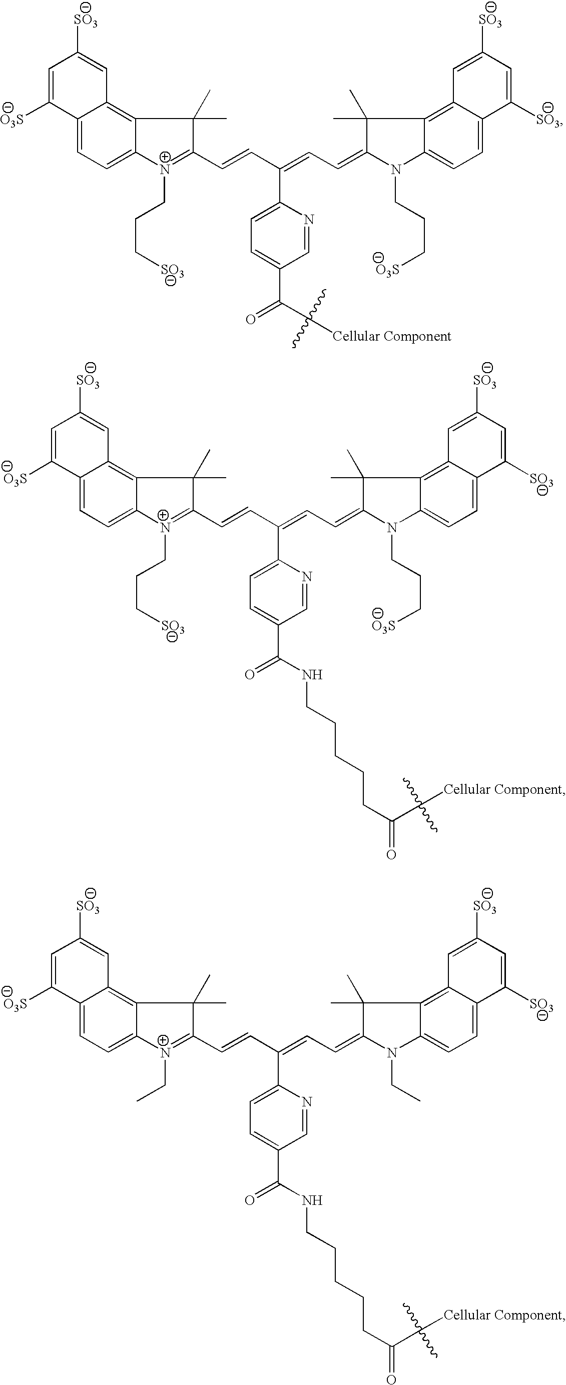

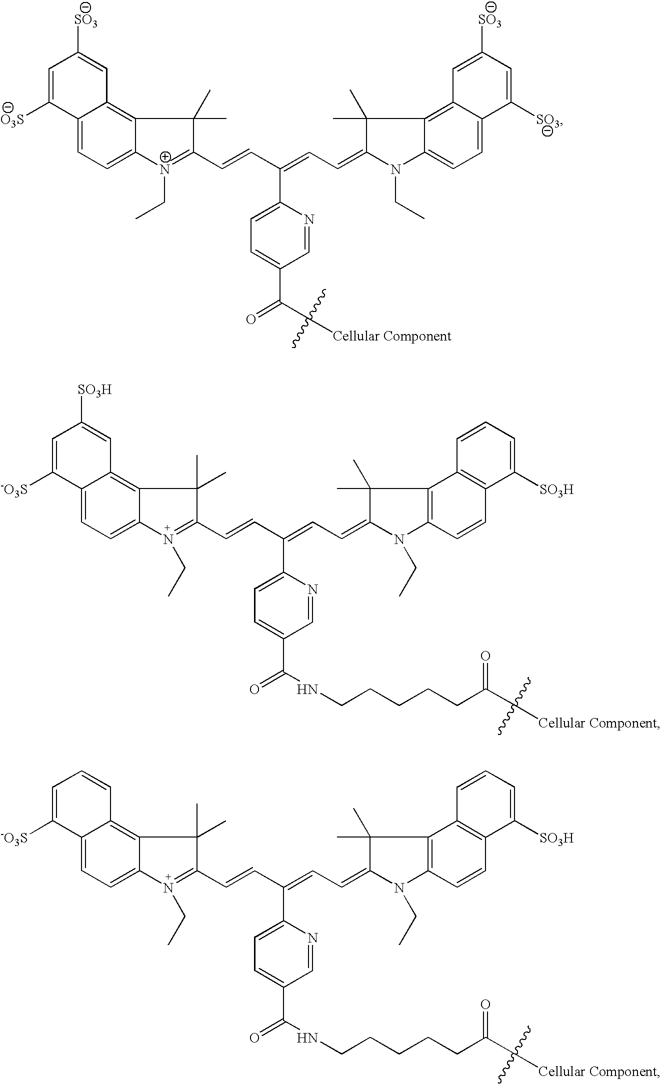

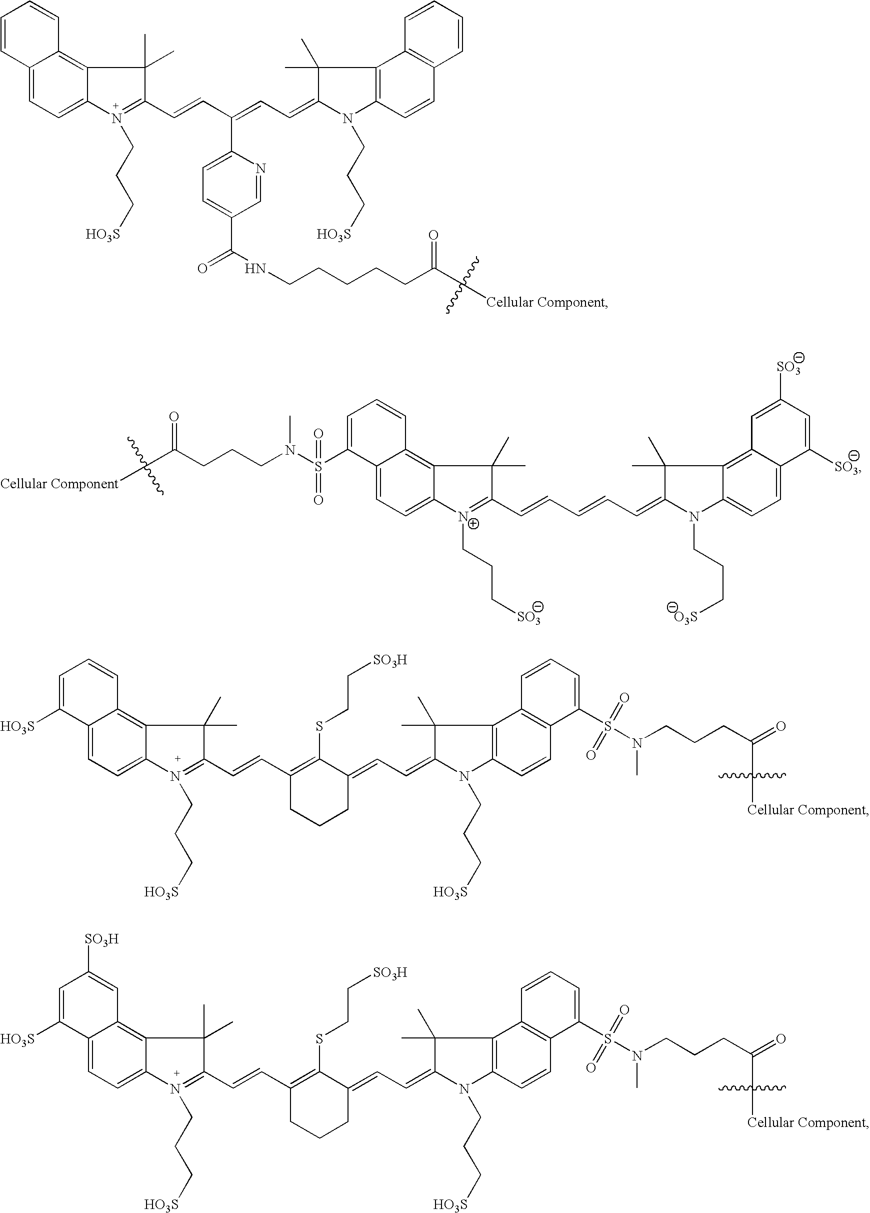

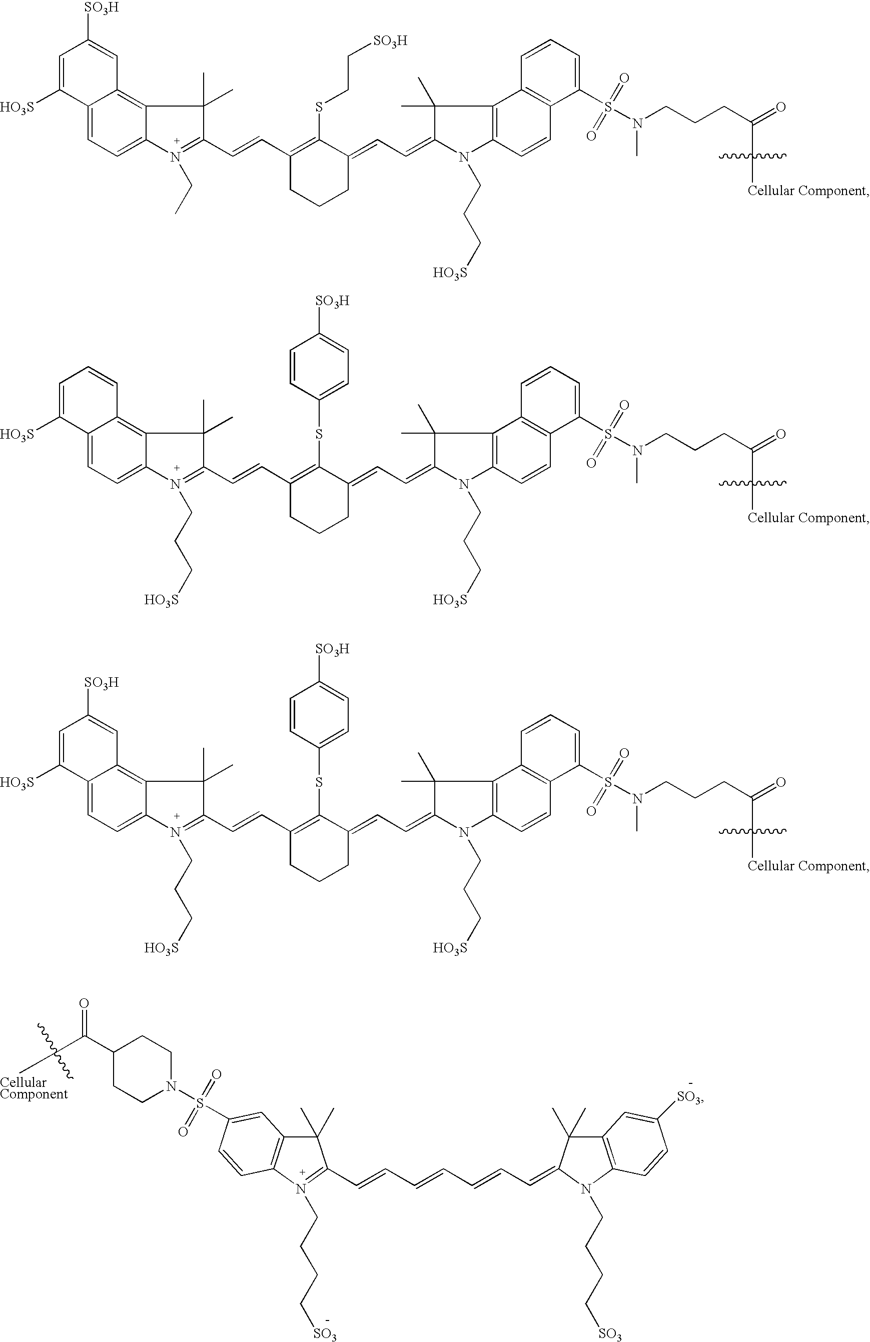

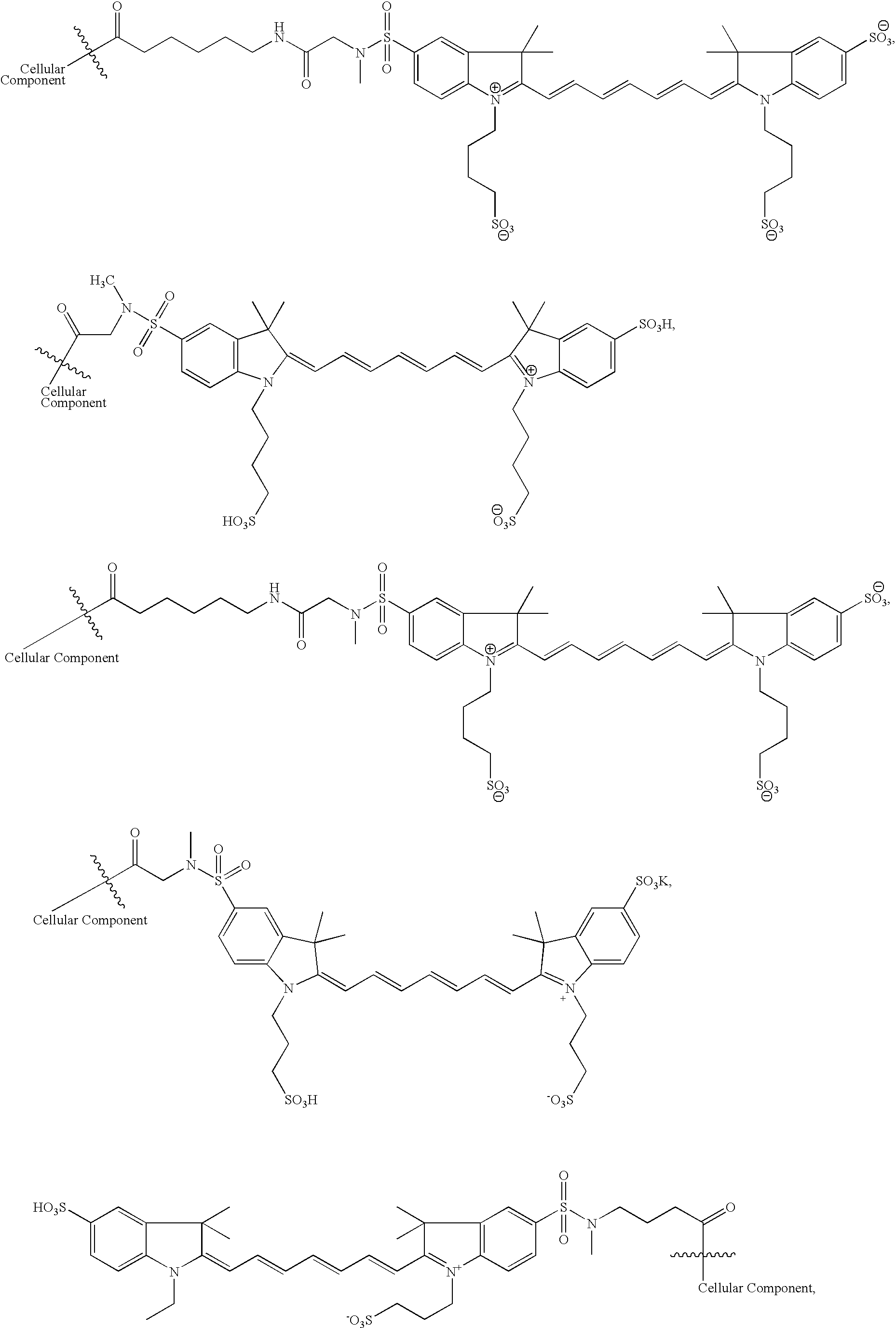

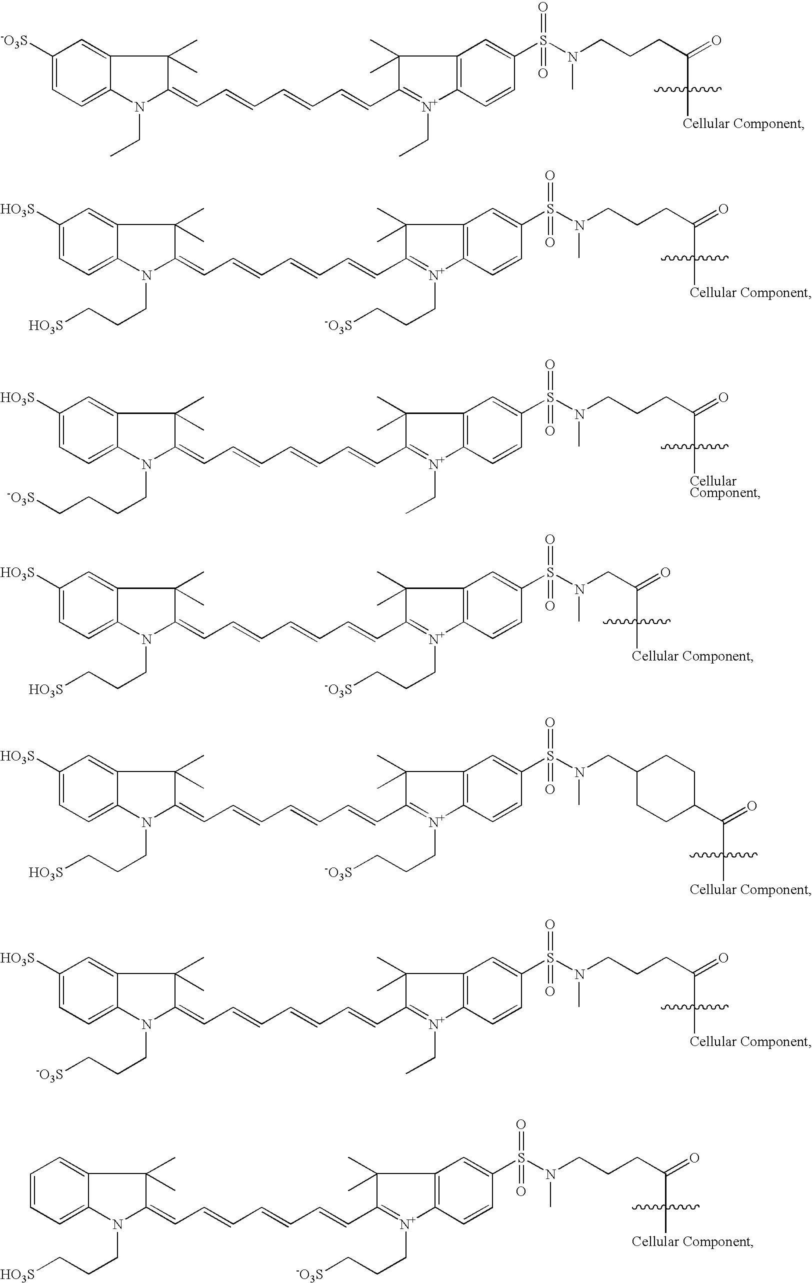

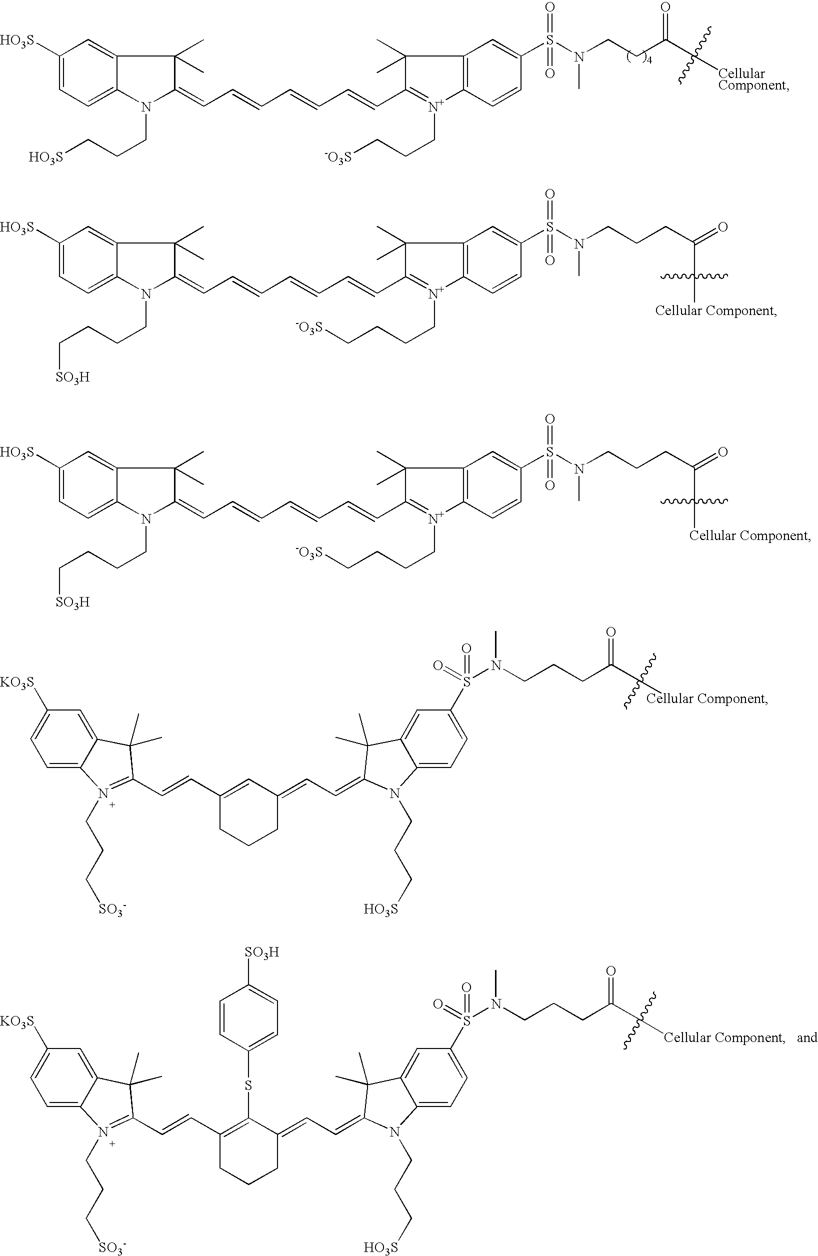

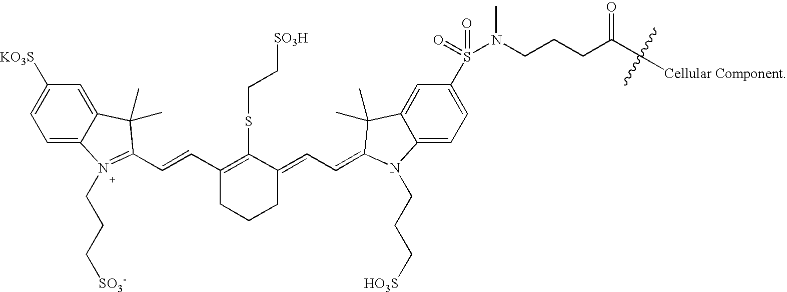

16. The method of claim 1, wherein the cells comprise primary cells.

17. The method of claim 1, wherein the cells are selected from the group consisting of T-cells, B-cells, tumor cells, stem cells, bacterial cells, macrophages, lymphocytes, monocytes, and splenocytes.

18. The method of claim 1, wherein step (b) and/or step (c) is/are performed using at least one of: an endoscope, catheter, planar system, reflectance system, tomographic system, optical imaging system and/or an intraoperative microscope.

19. A method of detecting and/or monitoring a disease comprising performing the in vivo imaging method of claim 1.

20. The method of claim 19, wherein the disease is selected from the group consisting of bone disease, cancer, cardiovascular disease, environmental disease, dermatological disease, immunologic disease, inherited disease, infectious disease, inflammatory disease, metabolic disease, neurodegenerative disease, ophthalmic disease, and respiratory disease.

21. A method of detecting and/or monitoring cell-based therapies comprising performing the in vivo imaging method of claim 1.

22. A method of making a plurality of viable near-infrared fluorochrome labeled cells for use in in vivo imaging comprising:
a) contacting a plurality of viable cells with near-infrared fluorochrome molecules under conditions to (i) covalently link the cells with at least one near-infrared fluorochrome, and (ii) maintain the viability of the cells, wherein the cells have substantially the same function and/or viability as the cells prior to labeling; and
b) removing unbound near-infrared fluorochrome molecules thereby to produce a plurality of viable near-infrared fluorochrome labeled cells.

23. The method of claim 22, wherein, step (a) is performed such that the reaction occurs in a solution substantially free of organic solvent.

24. The method of claim 23, wherein the solution is substantially free of DMSO.

25. The method of claim 22, wherein the cells are primary cells.

26. The method of claim 22, wherein the cells are selected from a group consisting of B-cells, T-cells, immune cells, tumor cells, stem cells, bacterial cells, macrophages, lymphocytes, monocytes, and splenocytes.

27. The method of claim 22, wherein the near-infrared fluorochrome molecule is a carbocyanine dye.

28. (canceled)

29. The method of claim 22, wherein the near-infrared fluorochrome molecule is selected from the group consisting of Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, and DyLight647, HiLyte Fluor 680, HiLyte Fluor 750, IRDye800CW, IRDye 800RS, IRDDye 700DX, ADS780WS, and ADS832WS.

30. (canceled)

31. The method of claim 22, wherein the near-infrared fluorochrome molecule is selected from the group consisting of:

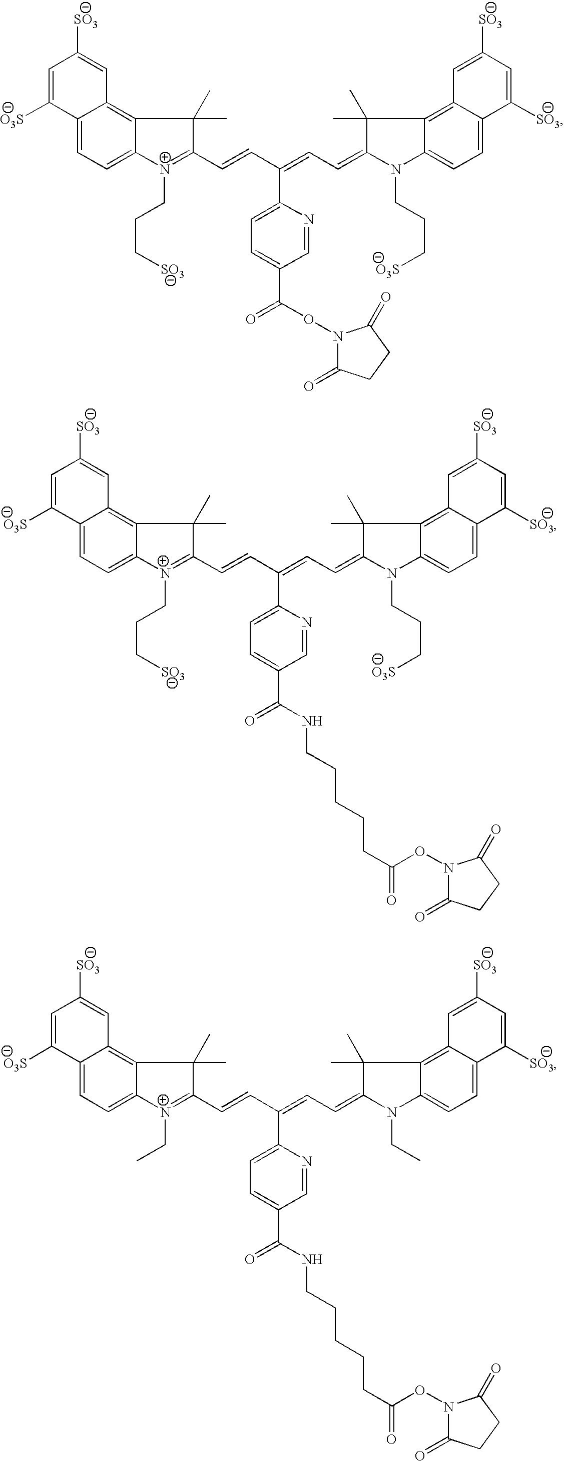

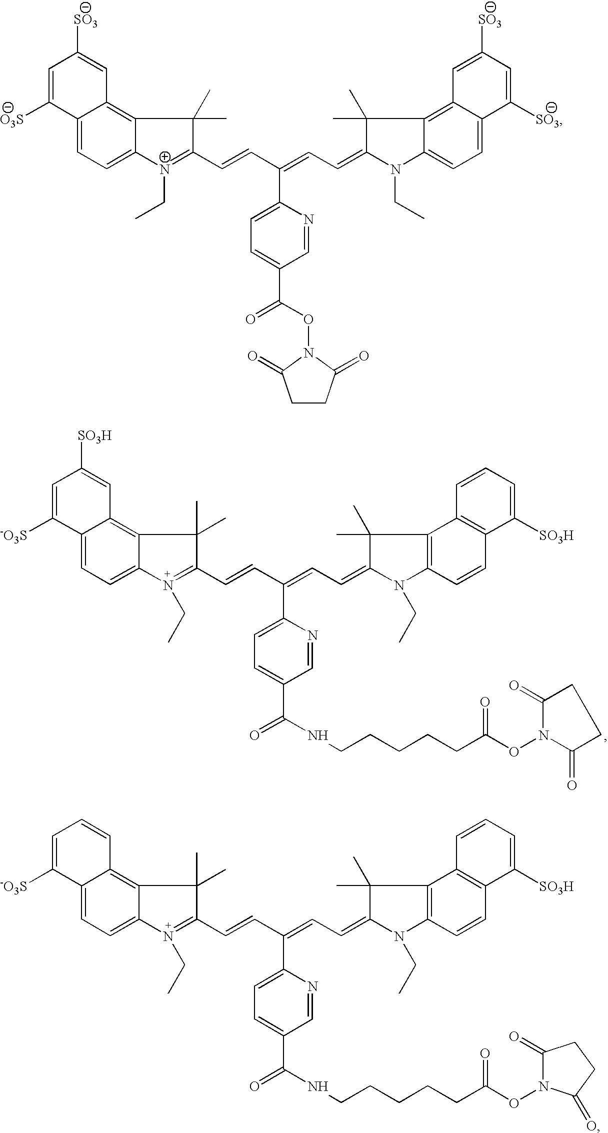

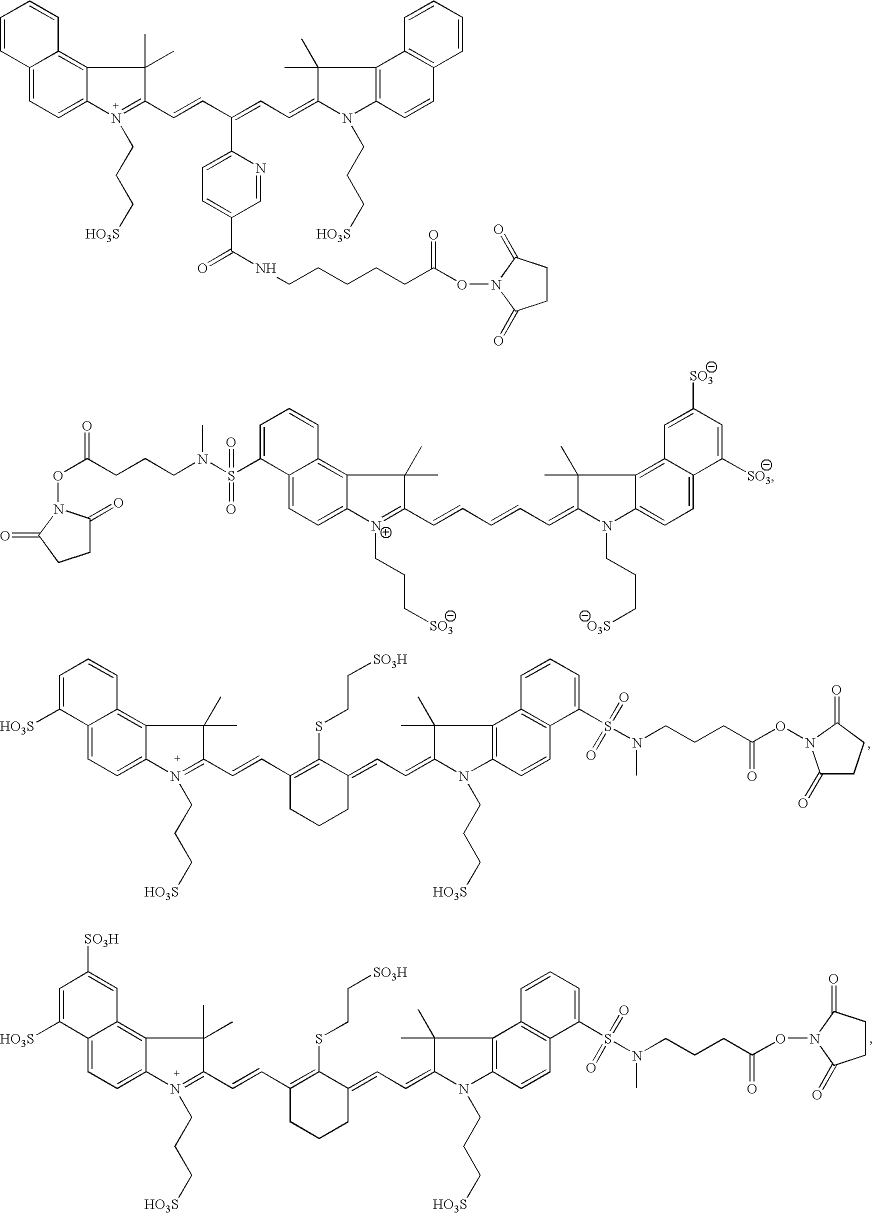

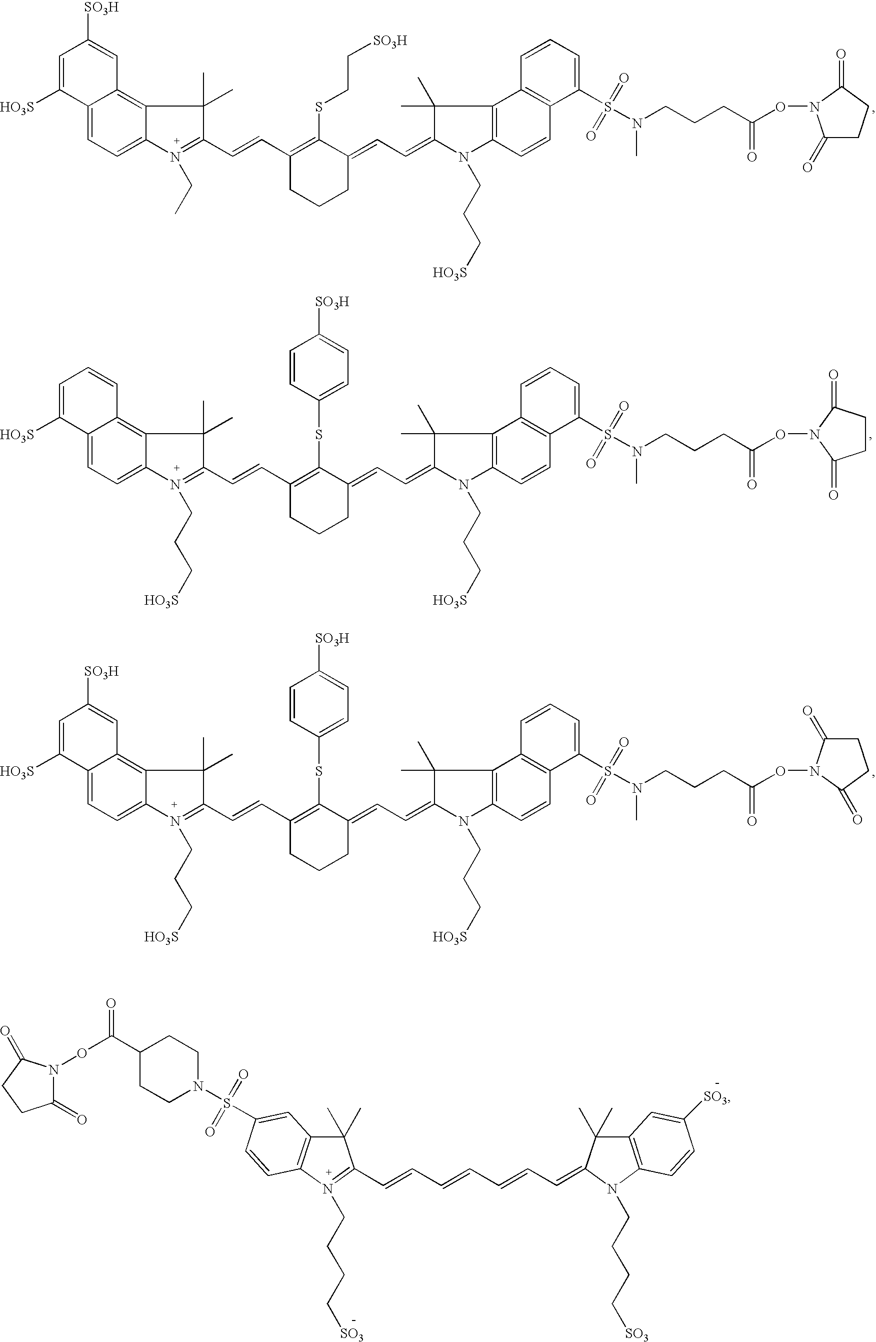

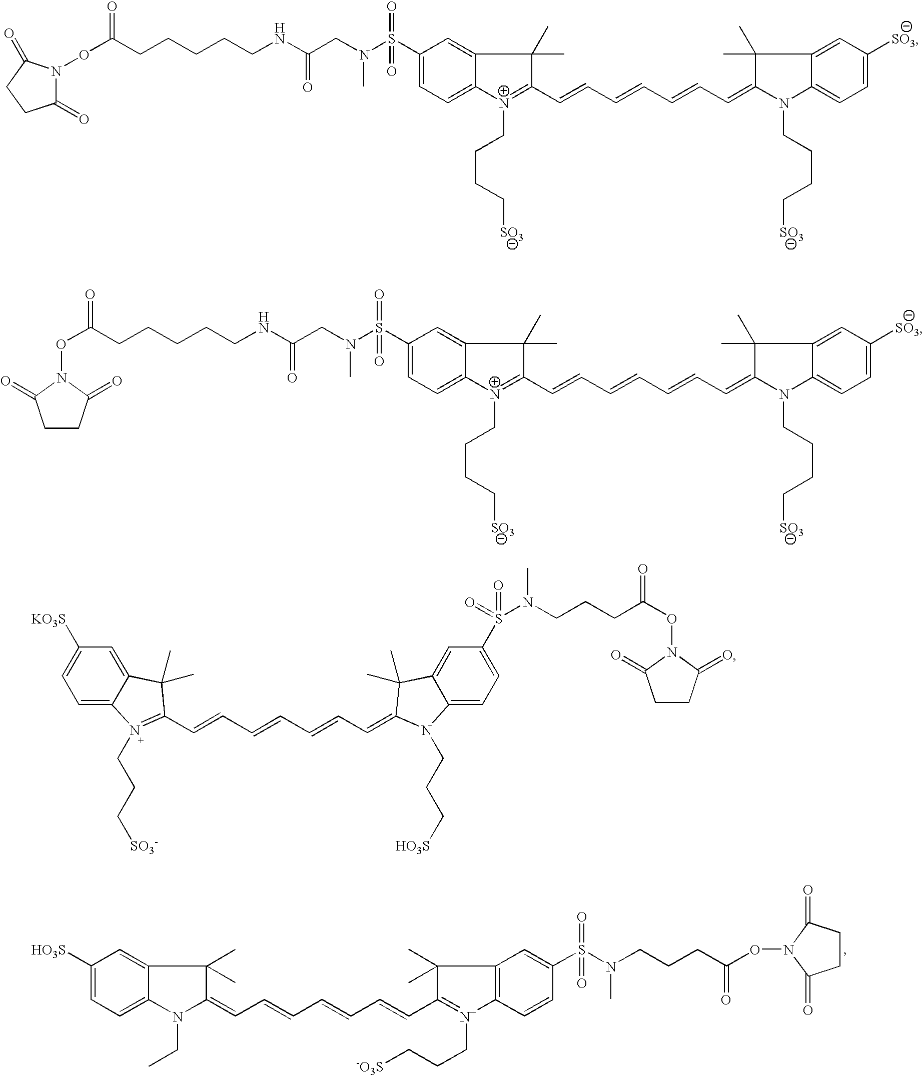

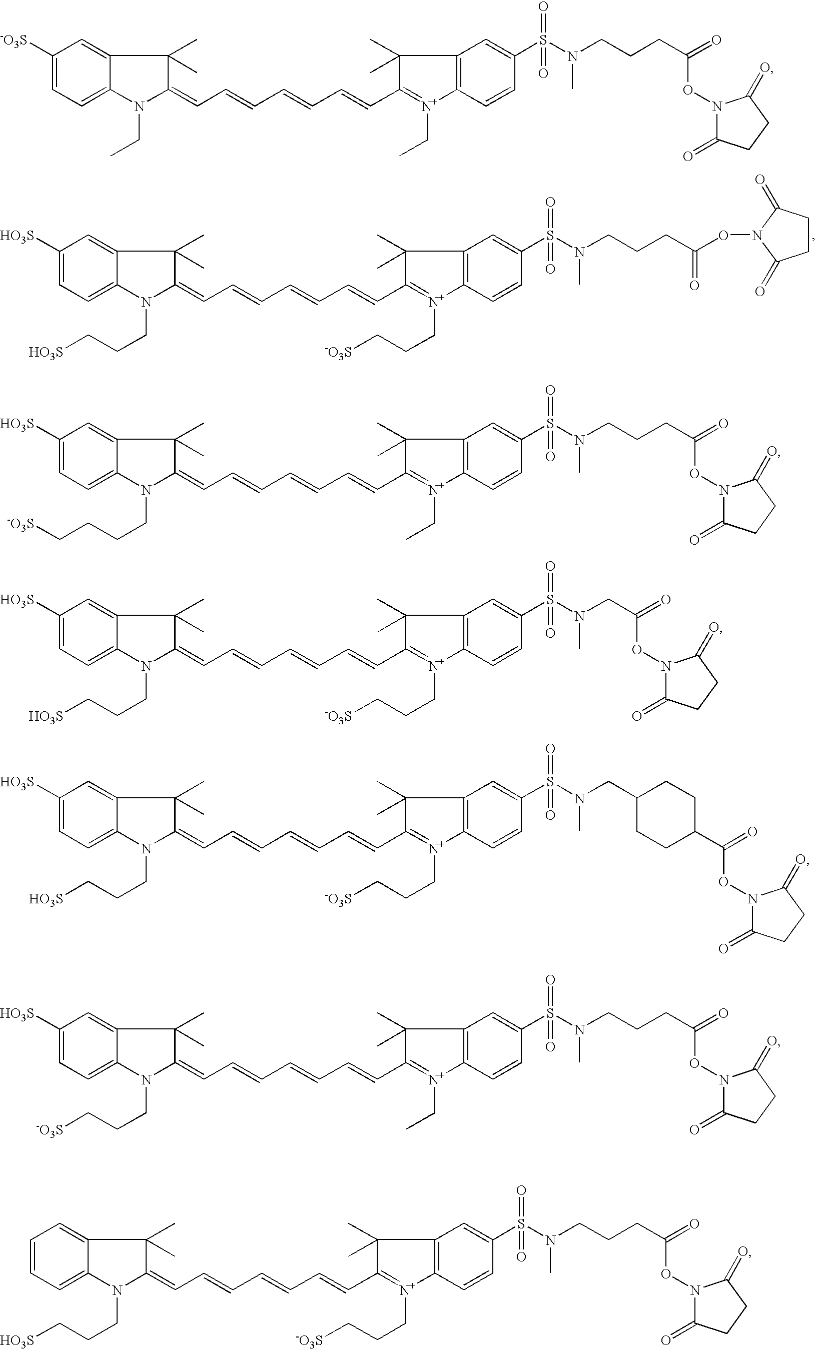

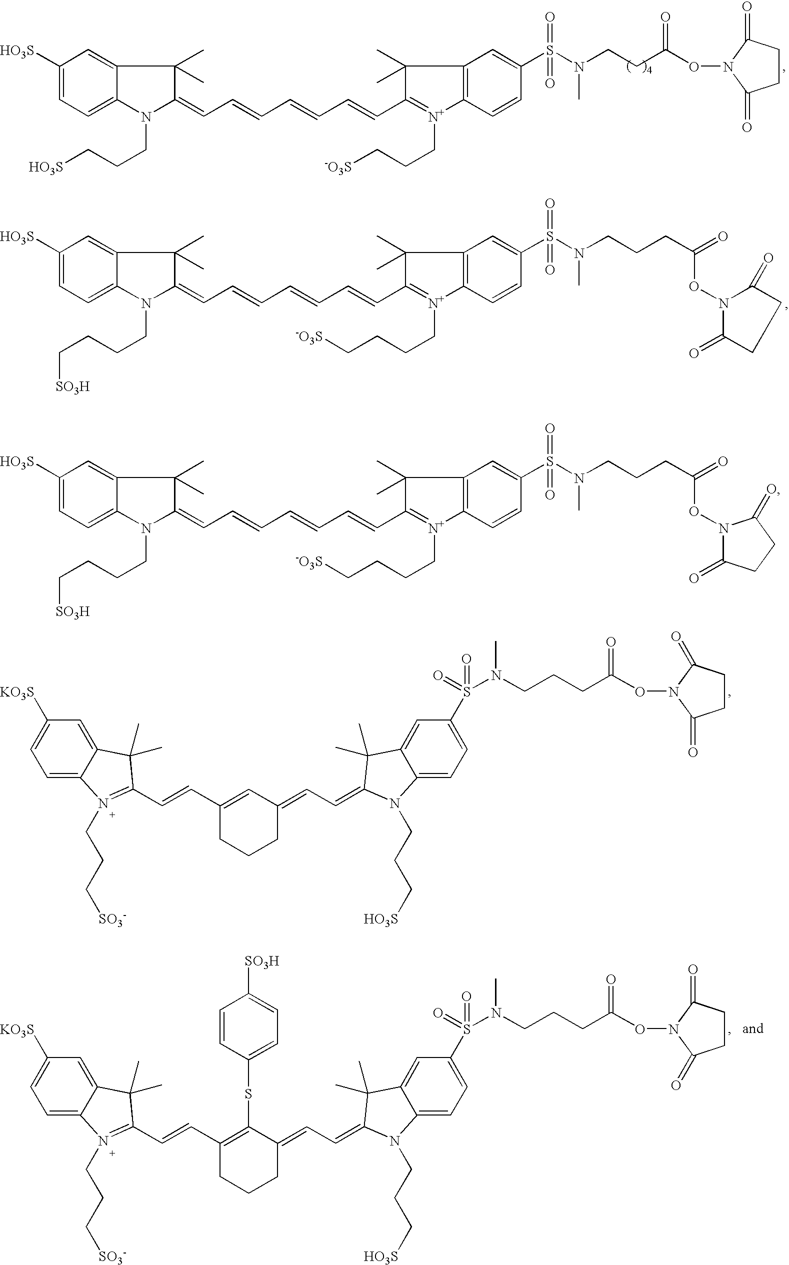

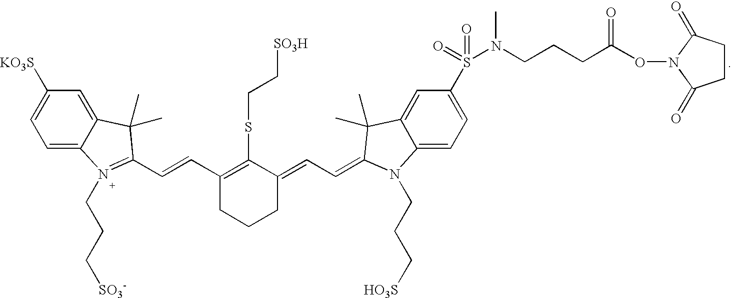

32-37. (canceled)

38. A composition for use in in-vivo imaging comprising a plurality of viable cells covalently linked to at least one near-infrared fluorochrome molecule, wherein the cells have substantially the same function and/or viability as the cells prior to labeling, wherein the near-infrared fluorochrome molecule is selected from the group consisting of:

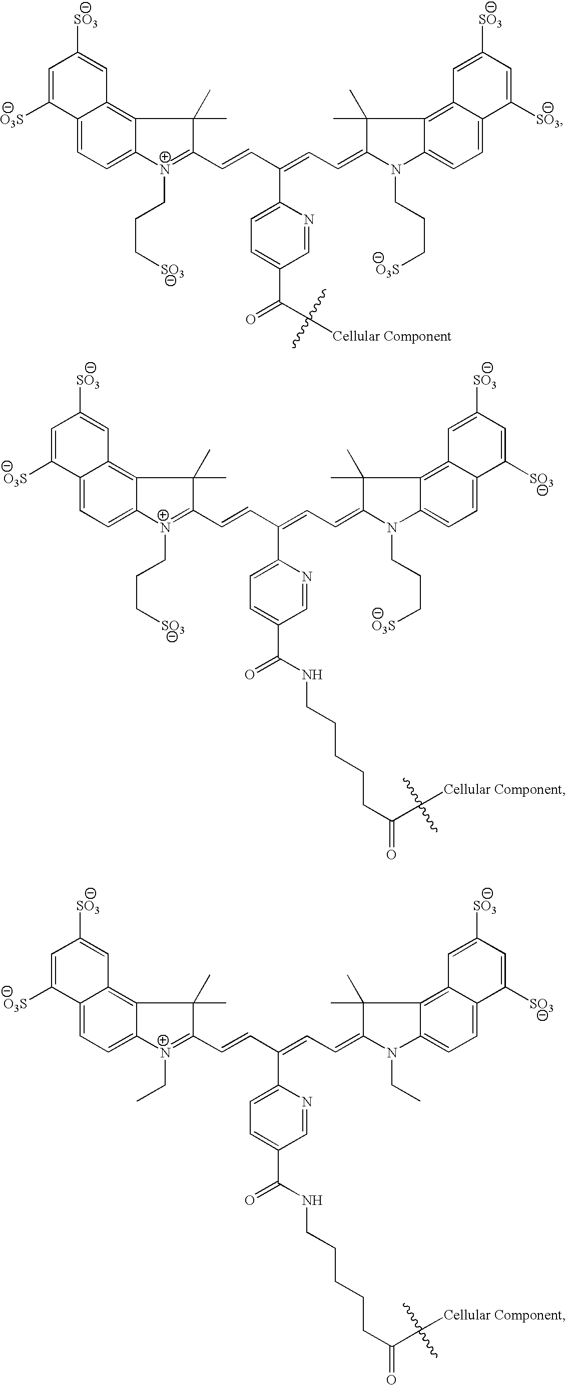

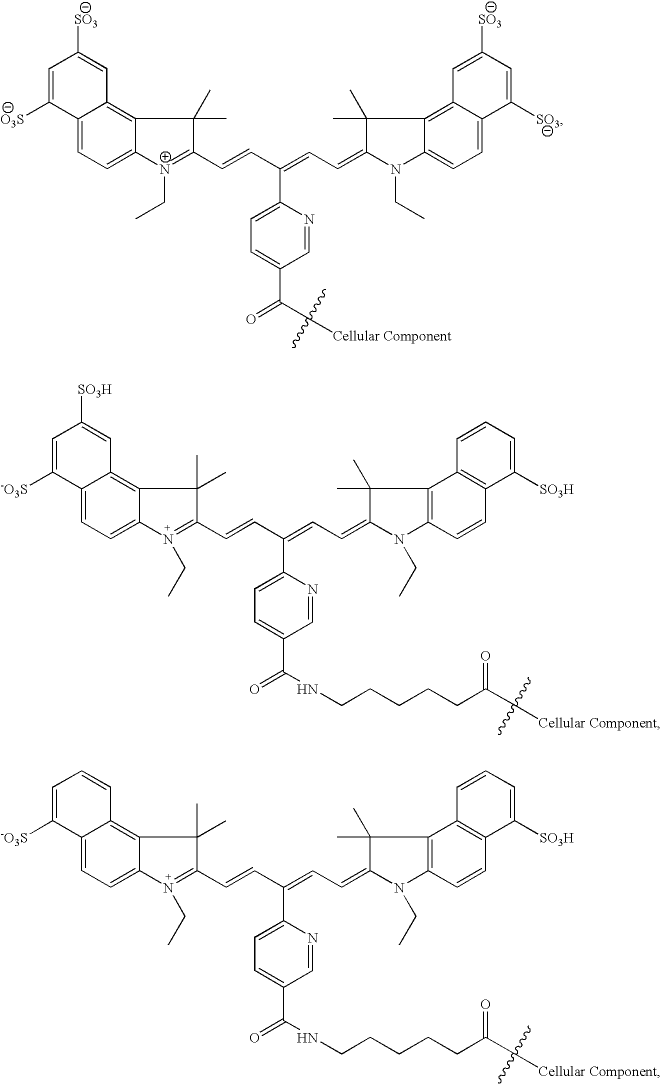

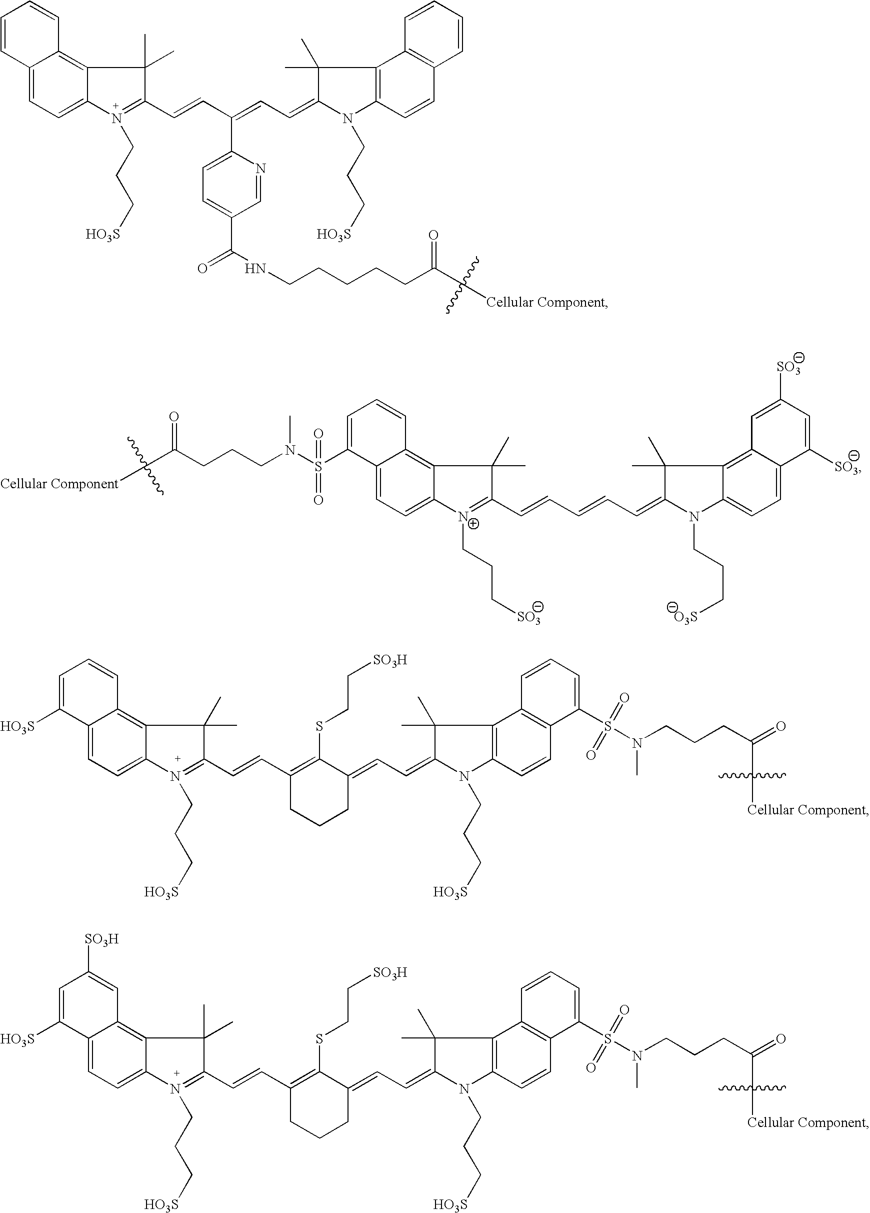

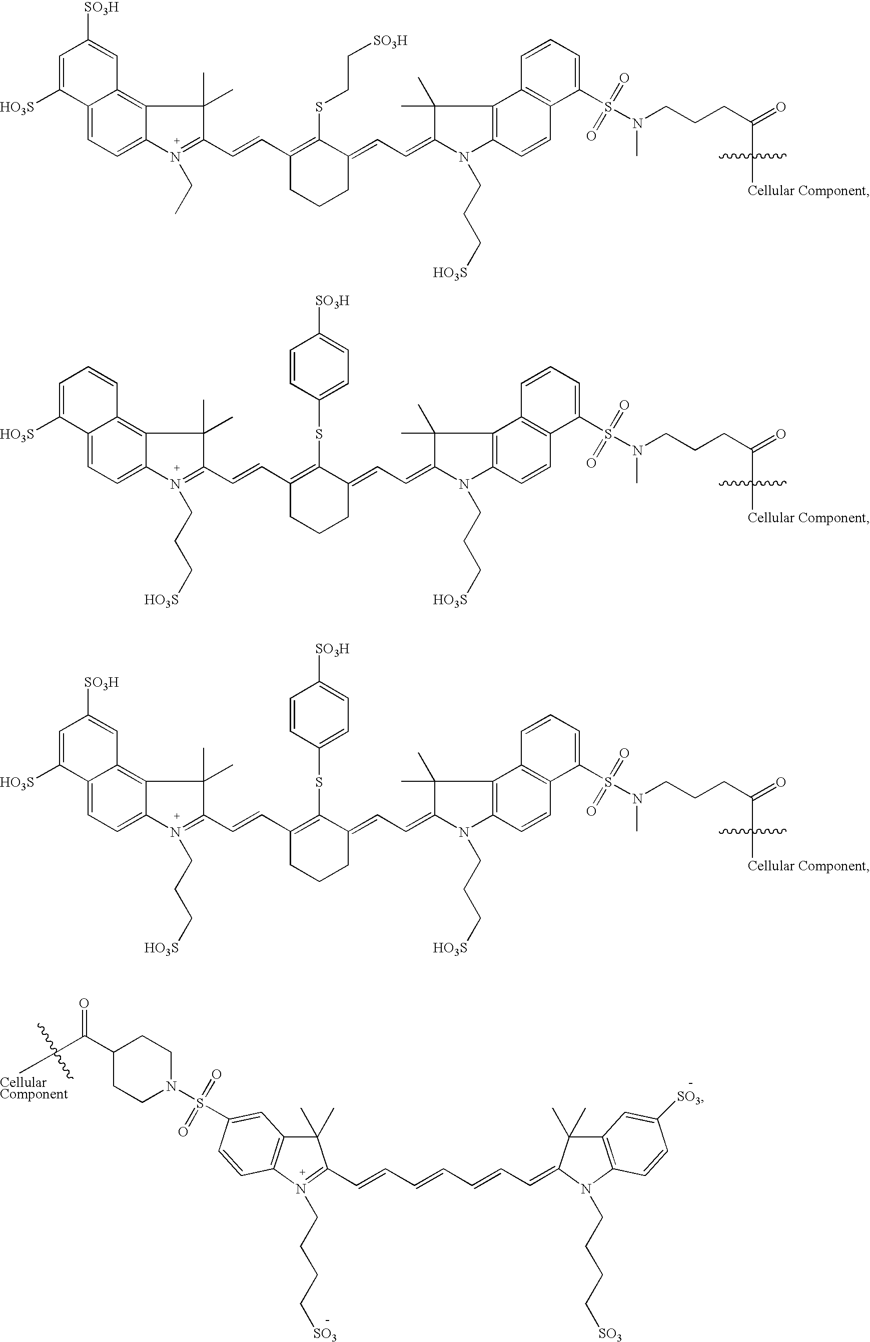

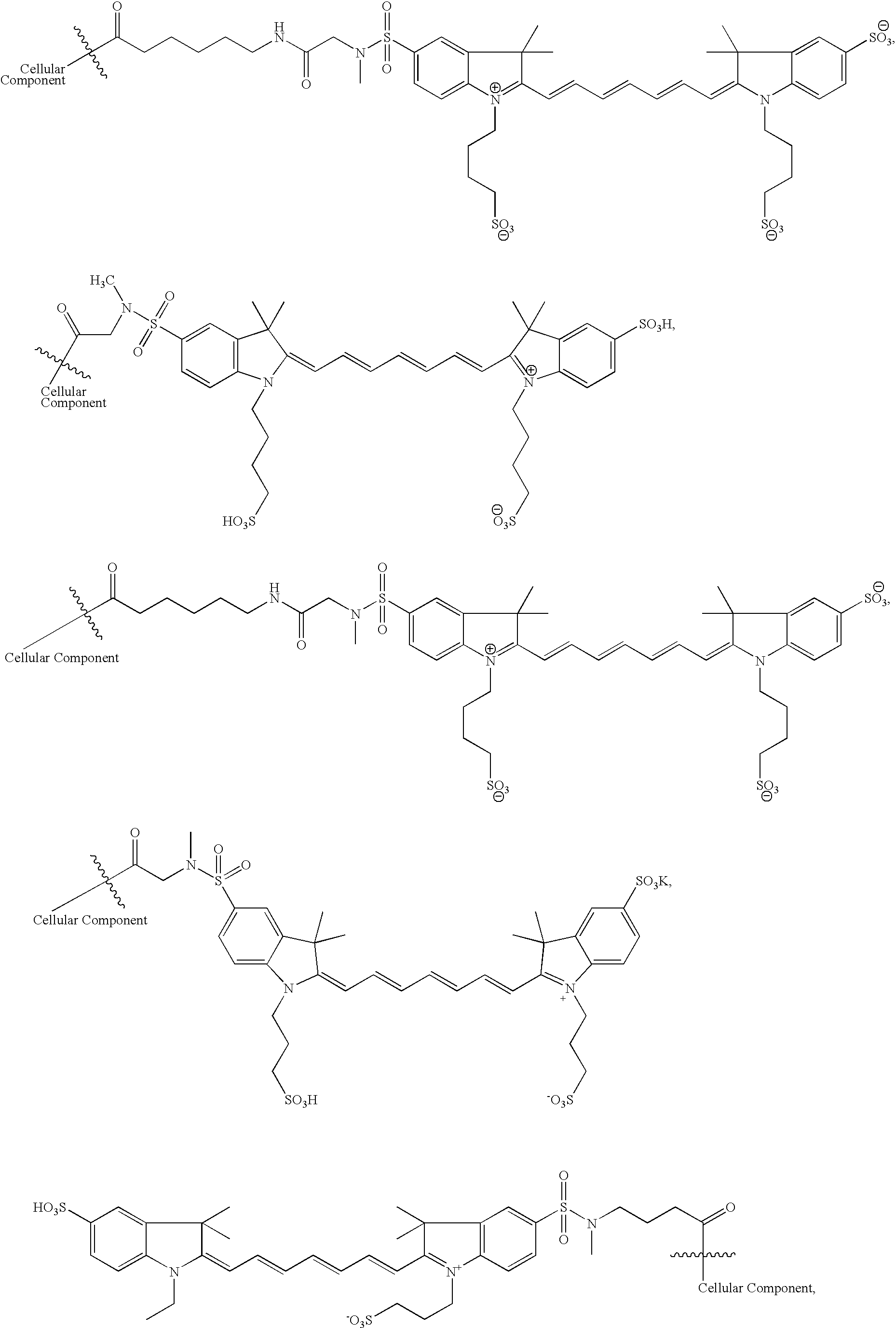

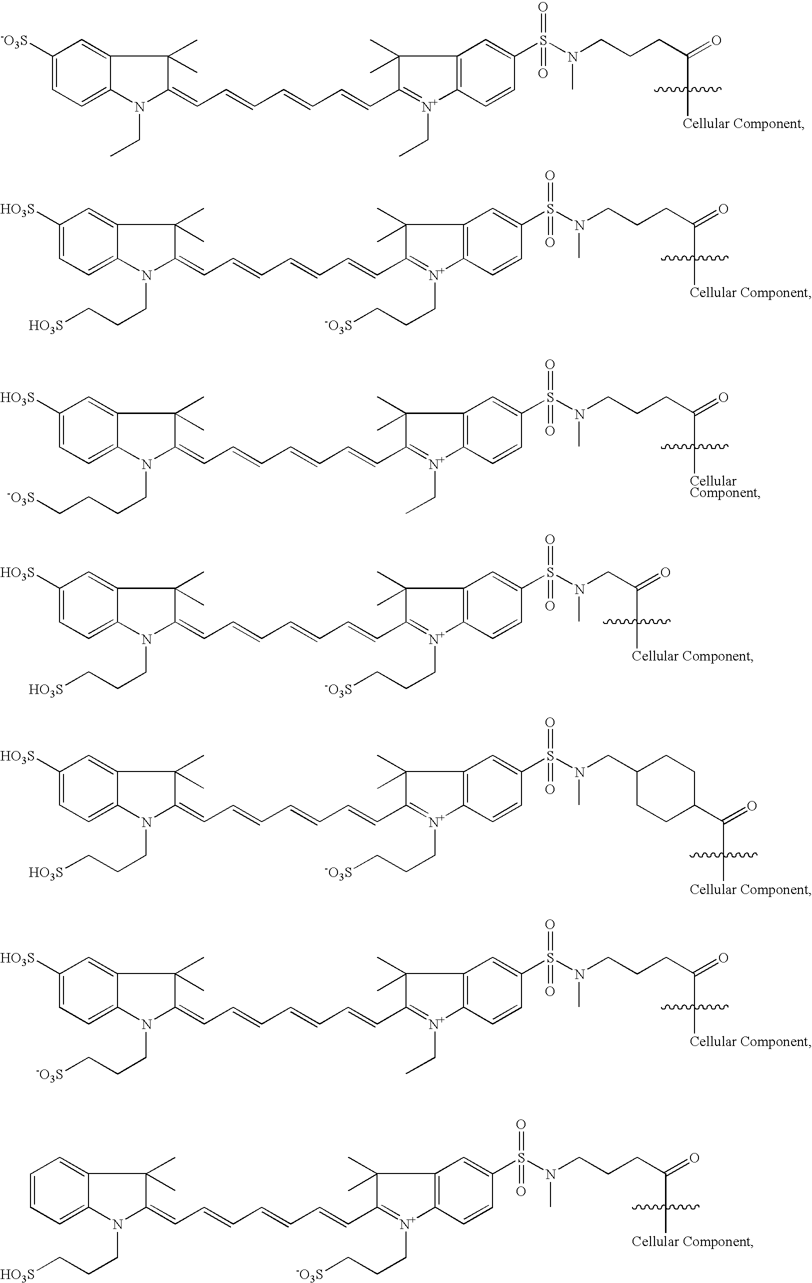

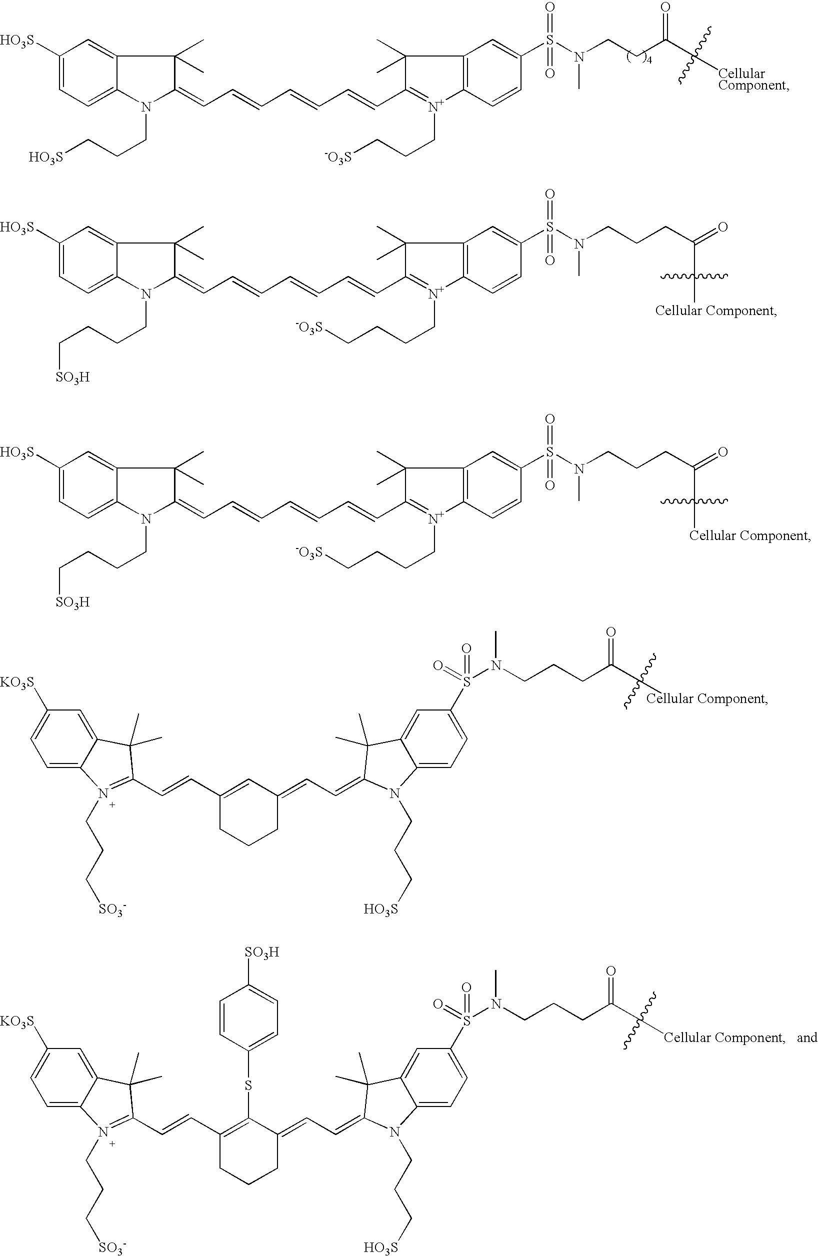

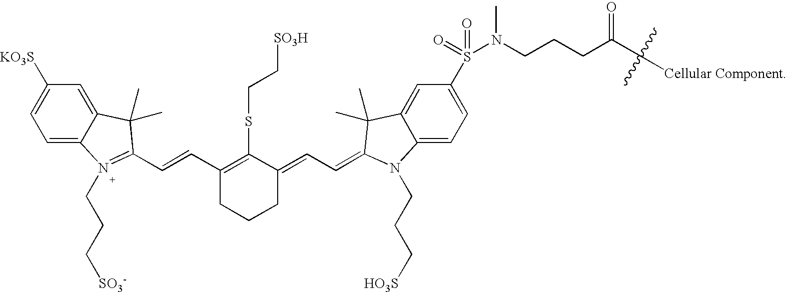

39-41. (canceled)
